(19) United States
(12) Patent Application Publication
(10) Pub. No.: US 2024/0270793 A1
(43) Pub. Date: Aug. 15, 2024

(54) COMPOSITIONS

(71) Applicant: UNIVERSITY OF SOUTHAMPTON, Southampton Hampshire (GB)

(72) Inventors: Ali TAVASSOLI, Southampton Hampshire (GB); Andrew T. BALL, Southampton Hampshire (GB)

(73) Assignee: UNIVERSITY OF SOUTHAMPTON, Southampton Hampshire (GB)

(21) Appl. No.: 18/037,402

(22) PCT Filed: Nov. 18, 2021
(86) PCT No.: PCT/GB2021/052989

§ 371 Date: May 17, 2023

(30) Foreign Application Priority Data
Nov. 19, 2020 (GB) 2018187.1

(51) Int. Cl. C07K 7/64; A61K 38/12

(52) U.S. Cl. CPC C07K 7/64; A61K 38/12

(57) ABSTRACT

The invention provides cyclic peptides that are able to disrupt the typical response to hypoxia and which have particular utility in the treatment of cancers and von Hippel-Lindau disease.

### FIELD

The invention is in the field of therapeutic agents suitable for use in treating diseases or conditions that involve the response to hypoxia.

### BACKGROUND

Hypoxia is a state of reduced oxygen concentration that can arise under normal conditions such as embryonic development, but also plays a key role in multiple pathological conditions, such as cardiac arrest, stroke and cancer.¹ Hypoxia has particular relevance in cancers as solid tumours contain hypoxic regions (pO₂≤2.5 mmHg)² that occur due to tumour cell growth exceeding the capacity of the surrounding vascular infrastructure. Hypoxia inducible factors (HIF) are heterodimeric transcription factors that assemble in hypoxia and reprogram gene expression to allow survival and growth of cells in a low oxygen microenvironment.³⁻⁵ The expression of several hundred genes has been directly linked to HIF-1 activation, and genomic analysis of HRE sequences estimates that HIF-1 mediates the expression of up to 1% of the genome.^{6,7} Whilst HIF activity impacts a diverse set of cellular pathways, the primary means by which hypoxic response is enacted is through the reprogramming of glucose metabolism, and the promotion of angiogenesis and proliferation. This response is believed to promote an aggressive phenotype and prolong tumour survival. As such, HIFs has long been proposed to be an attractive target for cancer therapy.

HIF is a heterodimeric transcription factor, which comprises of an oxygen-sensitive α subunit, and a constitutively-expressed β subunit (also known as the aryl hydrocarbon nuclear receptor translocator, ARNT). There are 3 isoforms of the HIF-α that bind to HIF-1β, with HIF-1α and HIF-2α being responsible for orchestrating hypoxia-response. The α-subunit of HIF is continually expressed but subject to post-translational modifications by oxygen-dependent proline hydroxylases (PHD). The hydroxylation of two prolines (P402 and P564 in HIF-1α) enables recognition by the von Hippel-Lindau protein and its associated E3 ligase complex, which triggers rapid ubiquitination and proteasomal degradation. Thus, HIF activity is acutely oxygen-sensitive, with HIF-1α having a half-life of less than 5 minutes in normoxia. HIF-1α is not degraded in hypoxia due to the absence of the molecular oxygen required for prolyl hydroxylation. The subsequent increase in HIF-α concentration causes it to translocate to the nucleus where it forms a dimeric complex with the constitutively expressed HIF-β to form the active HIF transcription factor. HIF binds to numerous hypoxia-response elements (HRE) present in the genome to reprogramme hypoxic cells to allow their survival and growth. The protein-protein interaction of the α and β subunit of HIF is a potential key point of therapeutic intervention, with recent successes in disrupting this interaction in HIF-2 translating into a potential cancer treatment programme.

Previous work in this area has largely focused on the specific targeting of one isoform over the other; this has largely been due to the presence of a small molecule cavity only present in the 2α PAS-B domain of HIF-2 that allows selective targeting of this isoform. HIF-1α is expressed ubiquitously, whereas HIF-2α and HIF-3α appear to be expressed in a more tissue-specific or environmentally conditional manner. Interestingly, HIF-1α and HIF-2α appear to have non-redundant roles that each produce distinct phenotypes due to their distinct target genes and in tissues where both isoforms are expressed, they have synergistic roles in promoting the hypoxic response.

The present invention seeks to provide inhibitors that are capable targeting both isoforms—HIF-1α and HIF-2α.

### SUMMARY OF PRESENT INVENTION

The present invention provides a series of cyclic peptides that inhibit the interaction of both HIF-1α and HIF-2α with HIf-1β by binding to the PAS-B domain of the a subunit of HIF. The compounds were identified from a SICLOPPS library of 3.2 million cyclic peptides and further optimized by using structure-activity relationship data to inform synthesis of analogous molecules containing non-natural amino acids. This led to a series of more potent, cell-permeable cyclic peptides that inhibit HIF assembly and subsequent hypoxia-response signalling in cells.

### DETAILED DESCRIPTION OF PRESENT INVENTION

### Definitions

As used herein, “alkyl” means a linear or branched alkane missing at least one hydrogen such that a bonding position is available, i.e. an alkyl group. Where a carbon chain length is not specified herein, “alkyl” means a C₁-C₁₀ alkyl group. In some embodiments, “alkyl” means a C₄-C₆ alkyl group. In other embodiments, “alkyl” means a C₁-C₃ alkyl group. Examples include methyl, ethyl, n-propyl and t-butyl. It may be monovalent, e.g. propyl, or divalent, e.g. propylene. A monovalent alkyl group may also be described by —CₙH₂ₙ₊₁ and a divalent alkyl group may also be described by —(CH₂)ₙ—, where n is independently selected from 1 to 10 for each substituent if not specified herein.

As used herein, “O-alkyl” means an alkyl group as defined above bonded to an oxygen atom, where said oxygen atom has a further available bonding position to form, for example, an ether via a C—O—C bond.

As used herein “halogen” or “halo” means an element from group 17 of the periodic table, preferably selected from fluorine, chlorine, bromine, and iodine.

As used herein “haloalkyl” means an alkyl group as defined above, which may be substituted with up to 10 halogen atoms or more preferably up to 5 halogens. For example, they may be substituted by 1, 2, 3, 4 or 5 halogen atoms. Preferably, the halogen is fluorine. Preferably the haloalkyl is selected from —CF₃, —CHF₂, and —CH₂F, further preferably —CF₃.

As used herein “aryl” or “aromatic group” means a monocyclic, bicyclic or tricyclic monovalent, divalent, trivalent or tetravalent (as appropriate) aromatic radical, such as phenyl, biphenyl, naphthyl, anthracenyl, which can be optionally substituted by preferably up to three substituents selected from the group comprising or consisting of halogen, C₁-C₃ alkyl, C₁-C₃ haloalkyl, NO₂, CN, OH or O—(C₁-C₃ alkyl). As used herein “aryl” or “aromatic group” includes aromatic heterocycles (i.e. cyclic compounds that has both carbon and non-carbon atoms forming the ring structure). Preferred non-carbon atoms (i.e. “heteroatoms”) are nitrogen, oxygen and sulphur. Preferably heterocycles contain one or two heteroatoms, preferably one. When there is more than one heteroatom in a heterocycle, the heteroatoms may be the same atom or different atoms. Examples of suitable aromatic heterocyclic rings containing one or more heteroatoms selected from O, S and N include furan, thiophene, pyrrole, imidazole, pyrazole, isoxazole, thiazole, isothiazole, pyridine, pyran, thiopyran, diazine, oxazine, thiazine, dioxine and dithiin.

Where an atom is identified herein, whether written or structurally indicated, said atom may be replaced by any known atomic isotopes of said atom, including stable and radioactive isotopes (i.e. variants of said atom differing in neutron number); for example, a deuterium atom may replace a hydrogen atom where a hydrogen atom is indicated. Synthetic methods for incorporating stable- and radio-isotopes are well-known in the art. Preferably, the atom is as identified herein.

As used herein, the above groups can be followed by the suffix -ene. This means that the group is divalent, i.e. a linker group. The linker (i.e. divalent) groups listed herein or in the claims are not ‘direction specific’. They can be reversed.

Compounds with which the invention is concerned, which may exist in one or more stereoisomeric form because of the presence of asymmetric atoms or rotational restrictions, can exist as a number of stereoisomers with R or S stereochemistry at each chiral centre or as atropisomers with R or S stereochemistry at each chiral axis. The invention includes all such enantiomers and diastereoisomers and mixtures thereof.

Where a chemical structure is shown, the accuracy of the structure takes preference over the compound name.

The full and abbreviated names of the amino acids or unnatural amino acids described herein will be known to the skilled person, however for the avoidance of doubt the below nomenclature is used herein:

| Abbreviation | Name | Structure |
|---|---|---|
| cys | cysteine | 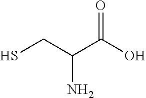 |
| leu | leucine | 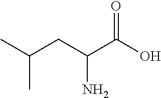 |
| arg | arginine | 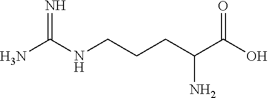 [SMILES: N=C(N)NCCCC(N)C(=O)O] |
| lys | lysine | 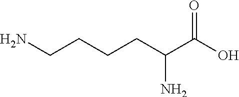 |
| ile | isoleucine | 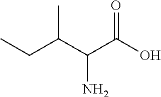 |
| phe | phenylalanine | 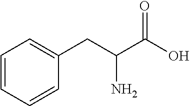 [SMILES: NC(Cc1ccccc1)C(=O)O] |
| val | valine | 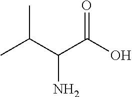 |
| ala | alanine | 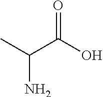 |
| met | methionine | 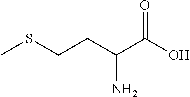 |
| ser | serine | 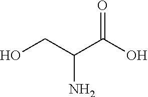 |
| thr | threonine | 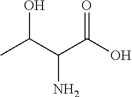 |
| tyr | tyrosine | 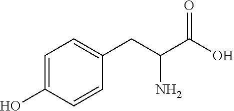 [SMILES: NC(Cc1ccc(O)cc1)C(=O)O] |
| D-Phe | D-phenylalinine | 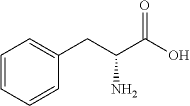 [SMILES: N[C@H](Cc1ccccc1)C(=O)O] |
| h-phe | homophenylalinine | 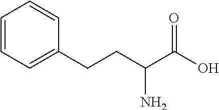 [SMILES: NC(CCc1ccccc1)C(=O)O] |
| phe(4-F) | 4-fluorophenylalanine | 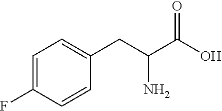 [SMILES: NC(Cc1ccc(F)cc1)C(=O)O] |
| phe(4-Cl) | 4-chlorophenylalanine | 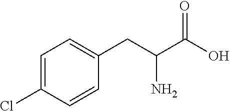 [SMILES: NC(Cc1ccc(Cl)cc1)C(=O)O] |
| phe(4-Br) | 4-bromophenylalanine | 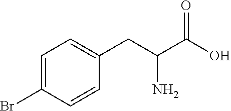 [SMILES: NC(Cc1ccc(Br)cc1)C(=O)O] |
| phe(4-I) | 4-iodophenylalanine | 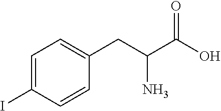 [SMILES: NC(Cc1ccc(I)cc1)C(=O)O] |
| phe(4-CF₃) | 4-triflurormethylphenyalinine | 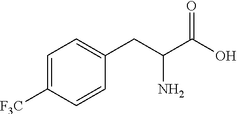 [SMILES: NC(Cc1ccc(C(F)(F)F)cc1)C(=O)O] |
| phe(4-Bz) | 4-benzoylphenyalinine | 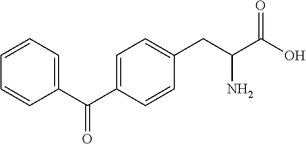 [SMILES: NC(Cc1ccc(C(=O)c2ccccc2)cc1)C(=O)O] |
| phe(4-NO₂) | 4-nitrophenylalanine | 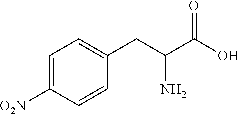 [SMILES: NC(Cc1ccc([N+](=O)[O-])cc1)C(=O)O] |
| phe(4-CN) | 4-cyanophenylalanine | 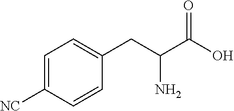 [SMILES: N#Cc1ccc(CC(N)C(=O)O)cc1] |
| ala(1-naph) | 1-naphthalenealanine | 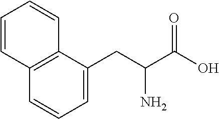 [SMILES: NC(Cc1cccc2ccccc12)C(=O)O] |
| gly(Ph) or (Phg) | phenylglycine | 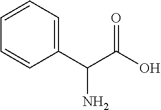 [SMILES: NC(C(=O)O)c1ccccc1] |
| tyr(OMe) Y(Me) or (me)tyr | O-methyltyrosine | 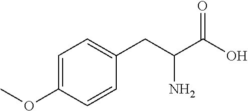 |
| h-leu | homoleucine | 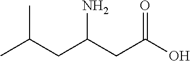 |
| n-leu | norleucine | 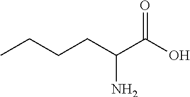 |
| pen | penicillamine | 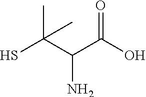 |
| pen(S-Py) | 2-pyridinethiolpenicillamine | 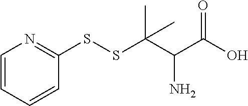 [SMILES: CC(C)(SSc1ccccn1)C(N)C(=O)O] |
| h-cys | homocysteine | 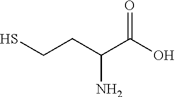 |
| h-cys(S-Py) | 2-pyridinethiolhomocysteine | 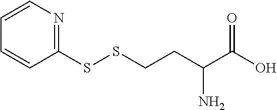 [SMILES: NC(CCSSc1ccccn1)C(=O)O] |
| cys(Me) | methylcysteine | 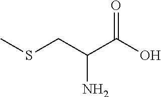 |
| ala(4-Py) or 4-Pal | 3-pyridin-4-ylalanine | 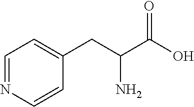 [SMILES: NC(Cc1ccncc1)C(=O)O] |
| n-val or nva | norvaline | 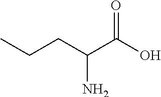 |
| aib | 2-aminoisobutyric acid | 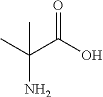 |
| abu | 2-aminobutanoic acid | 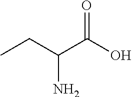 |

### Preferred Groups of the Invention

The invention provides a series of cyclic peptides which have a sequence that conforms to the following consensus sequence:

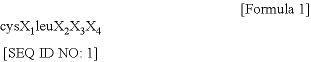

wherein:
cys is cysteine;
X₁ is selected from the group comprising or consisting of arginine (arg) or lysine (lys);
leu is leucine;
X₂ is selected from the group comprising or consisting of leu or isoleucine (ile);
X₃ is:

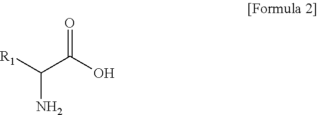

wherein:
   R₁ is C₂-C₆ linear or branched alkyl;
X₄ is:

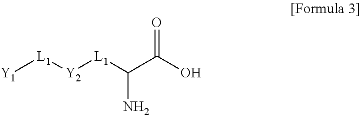

wherein:
   each L₁ is independently C₁-C₃ alkyl, a direct bond or C═O;
   Y₂ is a 5-12 membered aromatic group optionally substituted with halogen, C₁-C₃ alkyl, C₁-C₃ haloalkyl, NO₂, CN, OH or O—(C₁-C₃ alkyl); and
   Y₁ is H or a 5-12 membered aromatic group optionally substituted with halogen, C₁-C₃ alkyl, C₁-C₃ haloalkyl, NO₂, CN, OH or O—(C₁-C₃ alkyl);
provided that:
   when X₁ is arg, X₂ is leu and X₃ is ile; X₄ is not phenylalanine (phe), phe(4-Cl), phe(4-F), phe(4-NO2), or phe(4-CN); and
when X₁ is lysine (lys), X₂ is leu and X₃ is ile; X₄ is not (phe), phe(4-Cl), phe(4-F), phe(4-NO2), or phe(4-CN).

In one embodiment, R₁ is preferably a C₄-C₅ linear or branched alkyl, further preferably selected from the group comprising or consisting of —C₄H₉, —CH₂CH(CH₃)₂, —CH(CH₃)(C₂H₅), —CH₂CH₂CH(CH₃)₂.

In one embodiment, L₁ is preferably a C₁-C₂ alkyl, a direct bond or C═O.

In one embodiment, Y₂ is preferably a 6-10 membered aromatic group, further preferably selected from phenyl or naphthyl.

In one embodiment, the aromatic group of Y₂ is preferably substituted with a substituent from the group comprising or consisting of F, Cl, I, CF₃, CN, OH, OMe, and NO₂. In another embodiment, the aromatic group of Y₂ is unsubstituted.

In one embodiment, Y₁ is preferably H or phenyl, wherein the phenyl is preferably unsubstituted.

In one embodiment, X₃ is selected from the group comprising or consisting of leu, ile, h-leu, or abu.

In one embodiment, X₄ is selected from the group comprising or consisting of phe, phe(4-I), phe(4-Br), phe(4-CF₃), phe(4-Cl), h-phe, ala(1-naph), phe(4-F), phe(4-Bz), phe(4-NO₂), phe(4-CN), gly(Ph), D-phe, tyr(OMe), 4-Pal.

It will be clear to the skilled person that in some embodiments the peptide has the sequence:

[SEQ ID NO: 2]
cys arg leu ile ile X₄,
[SEQ ID NO: 3]
cys arg leu leu ile X₄,
[SEQ ID NO: 4]
cys lys leu ile ile X₄,
or
[SEQ ID NO: 5]
cys lys leu leu ile X₄.

In a preferred embodiment the cyclic peptide has the sequence:
cys arg leu ile ile phe [SEQ ID NO: 6], cys lys leu leu ile phe [SEQ ID NO:7] or cys arg leu ile ile 4-Pal [SEQ ID NO: 62], preferably cys arg leu ile ile phe [SEQ ID NO: 6].

The inventors found that modifying the phe at position 6 of SEQ ID NO: 6 to comprise unnatural amino acid substitutions resulted in cyclic peptides that either retained the same affinity for HIF1α PAS-B domain as the cyclic peptide of SEQ ID NO: 6, or had an increased affinity. Accordingly, in another preferred embodiment the cyclic peptide has the sequence cys arg leu ile ile X₄ [SEQ ID NO:2].

In any of the embodiments (i.e, SEQ ID NO: 1-6 for example), X₄ may be selected from the group comprising or consisting of phe, phe(4-I), phe(4-Br), phe(4-CF₃), phe(4-Cl), h-phe, ala(1-naph), phe(4-F), phe(4-Bz), phe(4-NO₂), Pal or phe(4-CN). Preferably X₄ is selected from the group comprising or consisting of phe, phe(4-I), phe(4-Br), phe(4-CF3), phe(4-Cl), h-phe, ala(1-naph), phe(4-F), phe(4-Bz), phe(4-NO₂). More preferably, X₄ is selected from phe(4-I) or phe(4-Bz), optionally wherein X₄ is phe(4-I).

In some embodiments the cyclic peptide has a sequence selected from the group comprising or consisting of:

[SEQ ID NO: 8]
cys arg leu ile ile phe,
[SEQ ID NO: 9]
cys arg leu ile ile phe(4-Br),
[SEQ ID NO: 10]
cys arg leu ile ile phe(4-CF₃),
[SEQ ID NO: 11]
cys arg leu ile ile phe(4-Cl),
[SEQ ID NO: 12]
cys arg leu ile ile (h-phe),
[SEQ ID NO: 13]
cys arg leu ile ile (ala(1-naph)),
[SEQ ID NO: 14]
cys arg leu ile ile phe(4-F),
[SEQ ID NO: 15]
cys arg leu ile ile phe(4-Bz),
[SEQ ID NO: 16]
cys arg leu ile ile phe(4-NO₂),
[SEQ ID NO: 17]
cys arg leu ile ile phe(4-CN),
[SEQ ID NO: 18]
cys arg leu ile ile(gly(Ph)),
[SEQ ID NO: 19]
cys arg leu ile ile (D-phe),
[SEQ ID NO: 20]
cys arg leu ile ile tyr,
[SEQ ID NO: 48]
cys arg leu ile ile phe(4-I),
and
[SEQ ID NO: 62]
cys arg leu ile ile 4-Pal

In a preferred embodiment, the cyclic peptide has a sequence selected from the group comprising or consisting:

[SEQ ID NO: 8]
cys arg leu ile ile phe,
[SEQ ID NO: 9]
cys arg leu ile ile phe(4-Br),
[SEQ ID NO: 10]
cys arg leu ile ile phe(4-CF₃),
[SEQ ID NO: 11]
cys arg leu ile ile phe(4-Cl),
[SEQ ID NO: 12]
cys arg leu ile ile (h-phe),
[SEQ ID NO: 13]
cys arg leu ile ile (ala(1-naph)),
[SEQ ID NO: 14]
cys arg leu ile ile phe(4-F),
[SEQ ID NO: 15]
cys arg leu ile ile phe(4-Bz),
[SEQ ID NO: 16]
cys arg leu ile ile phe(4-NO₂),
[SEQ ID NO: 17]
cys arg leu ile ile phe(4-CN),
[SEQ ID NO: 48]
cys arg leu ile ile phe(4-I),
and
[SEQ ID NO: 62]
cys arg leu ile ile 4-Pal

A more preferred cyclic peptide has a sequence selected from the group comprising or consisting:

[SEQ ID NO: 8]
cys arg leu ile ile phe,
[SEQ ID NO: 9]
cys arg leu ile ile phe(4-Br),
[SEQ ID NO: 10]
cys arg leu ile ile phe(4-CF₃),
[SEQ ID NO: 11]
cys arg leu ile ile phe(4-Cl),
[SEQ ID NO: 12]
cys arg leu ile ile (h-phe),
[SEQ ID NO: 13]
cys arg leu ile ile (ala(1-naph)),
[SEQ ID NO: 14]
cys arg leu ile ile phe(4-F),
[SEQ ID NO: 15]
cys arg leu ile ile phe(4-Bz),
and
[SEQ ID NO: 16]
cys arg leu ile ile phe(4-NO₂).

The inventors also found that modifying the ile at position 5 of SEQ ID NO: 6 to comprise particular unnatural amino acids also generated cyclic peptides with useful affinity to HIF-1α PAS-B domain. Accordingly, in some embodiments, the cyclic peptide has a sequence selected from the group comprising or consisting of:

[SEQ ID NO: 63]
cys arg leu ile (h-leu) phe,
[SEQ ID NO: 23]
cys arg leu ile leu phe,
and
[SEQ ID NO: 69]
cys arg leu ile abu phe
[SEQ ID NO: 63]
preferably, cys arg leu ile h-leu phe.

In one embodiment the cyclic peptide has the sequence cys arg leu ile h-leu phe(4-I) [SEQ ID NO: 71].

In a preferred embodiment, the cyclic peptide has the sequence cys arg leu ile ile phe(4-F) [SEQ ID NO: 14].

It will be clear to the skilled person that any of the residues in the cyclic peptide can be a D or an L amino acid. Accordingly, in one embodiment any one or more of cys, X₁, leu, X₂, X₃, or X₄ is a D amino acid or derivative thereof. In another embodiment, any one or more of cys, X₁, leu, X₂, X₃, X₄ is an L amino acid or derivative thereof. In another embodiment, the cyclic peptide comprises both L and D amino acids or derivatives thereof.

As described above, the cyclic peptides of the present invention are considered to be useful in inhibiting the interaction between HIF-α and HIf-1β.

Accordingly, in one embodiment the cyclic peptide is an inhibitor of the interaction between HIF-2α and HIF-1β. In one embodiment the cyclic peptide is capable of binding to HIF-2α, for example is capable of binding to recombinantly expressed PAS-B domain of HIF-2α.

In another or the same embodiment, the cyclic peptide is an inhibitor of the interaction between HIF-1α and HIF-1β. In one embodiment the cyclic peptide is capable of binding to HIF-1α, for example is capable of binding to recombinantly expressed PAS-B domain of HIF-1α.

It is preferred if the cyclic peptide is both an inhibitor of the interaction between HIF-2α and HIF-1β and of the interaction between HIF-1α and HIF-1β. Accordingly, in some embodiments the cyclic peptide is capable of binding to HIF-1α and HIF-2α, for example is capable of binding to recombinantly expressed PAS-B domain of HIF-2α and HIF-1α.

The skilled person will understand what is meant by “capable of binding to HIF-2α” and “capable of binding to HIF-1α”. The cyclic peptide may bind to any region of HIF-1α and/or HIF-2α. As described in the examples, in some embodiments the cyclic peptide binds to recombinantly expressed PAS-B domain of HIF-2α and or recombinantly expressed PAS-B domain of HIF-1α. See, for example, Example 1 and FIG. 1e.

It is even more preferred if the cyclic peptide binds to HIF-1α and HIF-2α with no or little bias, i.e. binds to HIF-1α and HIF-2α with the same or similar affinity.

The skilled person will recognise that there are multiple means to determine the binding affinity of the cyclic peptide to HIF-1α and HIF-2α or to recombinantly expressed PAS-B domain of both HIF-1α and 2α. For example, in one embodiment the affinity of determined using microscale thermophoresis, for example is determining against the recombinantly expressed PAS-B domain of both HIF-1α and 2α using microscale thermophoresis.

The skilled person will understand when the affinity to which the peptide binds to HIF-1α and the affinity with which the peptide binds to HIF-2α is sufficiently similar to render the peptide particular useful. In one embodiment the peptide binds to HIF-1α and HIF-2α with a similar affinity when the difference in affinity of binding to HIF-1α and HIF-2α is:
less than 60 μM, 55 μM, 50 μM, 45 μM, 40 μM, 35 μM, 30 μM, 25 μM, 20 μM, 18 μM, 16 μM, 15 μM, 14 μM, 13 μM, 12 μM, 11 μM, 10 μM, 9 μM, 8 μM, 7 μM, 6 μM, 5 μM, 4 μM, 3 μM, 2 μM, 1 μM, 0.8 μM, 0.6 μM, 0.5 μM, 0.4 μM, 0.3 μM, 0.2 μM, 0.1 μM; and/or
between 0.1 μM and 10 μM, 0.2 μM and 9 μM, 0.3 μM and 8 μM, 0.4 μM and 7 μM, 0.5 μM and 6 μM, 0.6 and 5.5 μM, 0.7 and 5 μM, 0.8 and 4.5 μM, 0.9 μM and 4 μM, 1 and 3.5 μM, 1.25 μM and 3 μM, 1.5 μM and 2.75 μM, 1.75 μM and 2.5 μM, 2 μM and 2.25 μM; and/or
between 0.1 μM and 60 μM, 0.2 μM and 55 μM, 0.3 μM and 50 μM, 0.4 μM and 45 μM, 0.5 μM and 40 μM, 0.6 μM and 35 μM, 0.7 μM and 30 μM, 0.8 μM and 25 μM, 0.9 μM and 20 μM, 1 μM and 18 μM, 2 μM and 16 μM, 3 μM and 15 μM, 4 μM and 14 μM, 5 μM and 13 μM, 6 μM and 12 μM, 7 μM and 11 μM, 8 μM and 10 μM.

As described above, the ability of a cyclic peptide to bind to HIF-1α and HIF-2α can be determined by determining the ability of the cyclic peptide to bind to recombinantly expressed PAS-B domain of HIF-1α and HIF-2α. Accordingly, in one embodiment the peptide binds to recombinantly expressed PAS-B domain of HIF-2α and HIF-1α with a similar affinity when the difference in affinity of binding to recombinantly expressed PAS-B domain of HIF-2α and HIF-1α is:
less than 60 μM, 55 μM, 50 μM, 45 μM, 40 μM, 35 μM, 30 μM, 25 μM, 20 μM, 18 μM, 16 μM, 15 μM, 14 μM, 13 μM, 12 μM, 11 μM, 10 μM, 9 μM, 8 μM, 7 μM, 6 μM, 5 μM, 4 μM, 3 μM, 2 μM, 1 μM, 0.8 μM, 0.6 μM, 0.5 μM, 0.4 μM, 0.3 μM, 0.2 μM, 0.1 μM; and/or
between 0.1 μM and 10 μM, 0.2 μM and 9 μM, 0.3 μM and 8 μM, 0.4 μM and 7 μM, 0.5 μM and 6 μM, 0.6 and 5.5 μM, 0.7 and 5 μM, 0.8 and 4.5 μM, 0.9 μM and 4 μM, 1 and 3.5 μM, 1.25 μM and 3 μM, 1.5 μM and 2.75 μM, 1.75 μM and 2.5 μM, 2 μM and 2.25 μM; and/or
between 0.1 μM and 60 μM, 0.2 μM and 55 μM, 0.3 μM and 50 μM, 0.4 μM and 45 μM, 0.5 μM and 40 μM, 0.6 μM and 35 μM, 0.7 μM and 30 μM, 0.8 μM and 25 μM, 0.9 μM and 20 μM, 1 μM and 18 μM, 2 μM and 16 μM, 3 μM and 15 μM, 4 μM and 14 μM, 5 μM and 13 μM, 6 μM and 12 μM, 7 μM and 11 μM, 8 μM and 10 μM,
   for example, wherein the affinity is determined against the recombinantly expressed PAS-B domain of both HIF-1α and 2α using microscale thermophoresis.

In some embodiments, the cyclic peptide binds to recombinantly expressed PAS-B domain of HIF-1α with an affinity of:
less than 10 μM, 9.5 μM, 9 μM, 8.5 μM, 8 μM, 7.5 μM, 7 μM, 6.5 μM, 6 μM, 5.5 μM, 5 μM, 4.5 μM, 4 μM, 3.5 μM, 3 μM, 2.5 μM, 2 μM, 1.5 μM, 1 μM, 0.9 μM, 0.8 μM, 0.7 μM, 0.6 μM, 0.5 μM, 0.4 μM, 0.3 μM, 0.2 μM, 0.1 μM; and/or
between 0.1 μM and 10 μM, 0.2 μM, and 9.5 μM, 0.3 μM and 9 μM, 0.4 μM and 8.5 μM, 0.5 μM and 8 μM, 0.6 μM and 7.5 μM, 0.7 μM and 7 μM, 0.8 μM and 6.5 μM, 0.9 μM and 6 μM, 1 μM and 5.5 μM, 1.5 μM and 5 μM, 2 μM and 5 μM, 2.5 μM and 4.5 μM, 3 μM and 4 μM; and/or
between 0.5 μM and 5 μM, 0.6 μM and 4.5 μM, 0.7 μM and 4 μM, 0.8 μM and 3.5 μM, 0.9 μM and 3 μM, 1 μM and 2.5 μM, 1.25 μM and 2.25 μM, 1.5 μM and 2 μM,
   for example, wherein the affinity is determined against the recombinantly expressed PAS-B domain of HIF-1α using microscale thermophoresis.

In some embodiments, the cyclic peptide binds to recombinantly expressed PAS-B domain of HIF-2α with an affinity of:
less than 10 μM, 9.5 μM, 9 μM, 8.5 μM, 8 μM, 7.5 μM, 7 μM, 6.5 μM, 6 μM, 5.5 μM, 5 μM, 4.5 μM, 4 μM, 3.5 μM, 3 μM, 2.5 μM, 2 μM, 1.5 μM, 1 μM, 0.9 μM, 0.8 μM, 0.7 μM, 0.6 μM, 0.5 μM, 0.4 μM, 0.3 μM, 0.2 μM, 0.1 μM; and/or
between 0.1 μM and 10 μM, 0.2 μM, and 9.5 μM, 0.3 μM and 9 μM, 0.4 μM and 8.5 μM, 0.5 μM and 8 μM, 0.6 μM and 7.5 μM, 0.7 μM and 7 μM, 0.8 μM and 6.5 μM, 0.9 μM and 6 μM, 1 μM and 5.5 μM, 1.5 μM and 5 μM, 2 μM and 5 μM, 2.5 μM and 4.5 μM, 3 μM and 4 μM; and/or
between 0.5 μM and 5 μM, 0.6 μM and 4.5 μM, 0.7 μM and 4 μM, 0.8 μM and 3.5 μM, 0.9 μM and 3 μM, 1 μM and 2.5 μM, 1.25 μM and 2.25 μM, 1.5 μM and 2 μM,
   for example, wherein the affinity is determined against the recombinantly expressed PAS-B domain of HIF-2α using microscale thermophoresis.

In some embodiments, the cyclic peptide binds to recombinantly expressed PAS-B domain of both HIF-1α and HIF-2α with an affinity of:
less than 10 μM, 9.5 μM, 9 μM, 8.5 μM, 8 μM, 7.5 μM, 7 μM, 6.5 μM, 6 μM, 5.5 μM, 5 μM, 4.5 μM, 4 μM, 3.5 μM, 3 μM, 2.5 μM, 2 μM, 1.5 μM, 1 μM, 0.9 μM, 0.8 μM, 0.7 μM, 0.6 μM, 0.5 μM, 0.4 μM, 0.3 μM, 0.2 μM, 0.1 μM; and/or
between 0.1 μM and 10 μM, 0.2 μM, and 9.5 μM, 0.3 μM and 9 μM, 0.4 μM and 8.5 μM, 0.5 μM and 8 μM, 0.6 μM and 7.5 μM, 0.7 μM and 7 μM, 0.8 μM and 6.5 μM, 0.9 μM and 6 μM, 1 μM and 5.5 μM, 1.5 μM and 5 μM, 2 μM and 5 μM, 2.5 μM and 4.5 μM, 3 μM and 4 μM; and/or
between 0.5 μM and 5 μM, 0.6 μM and 4.5 μM, 0.7 μM and 4 μM, 0.8 μM and 3.5 μM, 0.9 μM and 3 μM, 1 μM and 2.5 μM, 1.25 μM and 2.25 μM, 1.5 μM and 2 μM,
   for example, wherein the affinity is determined against the recombinantly expressed PAS-B domain of both HIF-1α and 2α using microscale thermophoresis.

It will be apparent to the skilled person that the cyclic peptides of the invention are useful in the preventing or reducing the response to hypoxia. Accordingly, in one embodiment the cyclic peptide of the invention prevents or reduces the hypoxia induced expression from a promoter that comprises one or more hypoxia-responsive elements under hypoxic conditions.

The skilled person will understand what is meant by “hypoxia induced expression”. Hypoxia is a state of reduced oxygen concentration that can arise under normal conditions such as embryonic development, and in, for example, the tumour microenvironment. Examples of hypoxic conditions include . The skilled person will also understand what is meant by a hypoxia-responsive element (HRE).

For example, as described in the examples, a peptide of the invention prevents or reduces hypoxia induced expression from a promoter that comprises one or more hypoxia-responsive elements when the peptide prevents or reduces expression of a reporter protein, for example a yellow fluorescent reporter protein (YFP) which is under the control of a promoter with three copies of the HRE sequence in a cell, for example in a HEK cell line, for example in the T-REx-293 cell line, where the cell is exposed to hypoxic conditions. In the absence of a cyclic peptide of the invention, hypoxic conditions would result in expression of the reporter (e.g. YFP) as it is under the control of a promoter that comprises HRE elements. In the presence of a cyclic peptide of the invention, this increase in expression is prevented. See for example FIG. 4.

It will be understood by the skilled person that the term “expression” includes the meaning of transcription of a non-coding gene into a non-coding RNA, for example into an miRNA or a siRNA; as well as including the meaning of transcription of a coding gene into a coding RNA/mRNA and subsequent translation into a peptide or polypeptide.

In one embodiment, the cyclic peptide reduces the hypoxia induced expression from a promoter that comprises one or more hypoxia-responsive elements under hypoxic conditions to less than 90%, 80%, 70%, 60%, 50%, 40%, 30%, 20%, 10%, 5%, 4%, 3%, 2%, 1% of the expression obtained in the absence of the cyclic peptide under hypoxic conditions. For example in one embodiment, the cyclic peptide reduces the hypoxia induced expression of a reporter protein, for example YFP, from a promoter that comprises one or more hypoxia-responsive elements, for example from a promoter that comprises three HRE sequences, under hypoxic conditions.

The cyclic peptides are considered to be particularly useful if they are able to disrupt the interaction between the α and β subunits of the HIF heterodimeric protein, i.e. prevent association or binding of the α and β subunits of the HIF heterodimeric protein. In one embodiment the cyclic peptides of the invention are able to disrupt the interaction between HIF-1α and HIF-1β. In the same or different embodiment, the cyclic peptides of the invention are able to disrupt the interaction between HIF-2α and HIF-1β. In a preferred embodiment, the cyclic peptides are able to disrupt the interaction between HIF-1α and HIF-1β, and between HIF-2α and HIF-1β.

The skilled person will understand how to determine whether a particular cyclic peptide is able to disrupt the interaction between two particular subunits. For example, various methods are used to determine protein-protein interactions, including yeast two hybrid assays and protein cross linking methods. The proximity ligation assay may also be used, whereby the interacting partner domains are targeted by separate primary and secondary antibodies. The secondary antibodies comprise PLA probes that contain short DNA strands. When in close proximity, i.e. where the interacting partner domains are contacting one another, the DNA strands can be amplified via rolling circle DNA synthesis. Identification of an amplification product indicates an interaction between the two partner domains. The proximity ligation assay can be performed in situ, for example in cells, for example in MCF-7 cells.

Accordingly, in one embodiment the cyclic peptide disrupts the interaction between HIF-1α and HIF-1β; between HIF-2α and HIF-1β; or between HIF-1α and HIF-1β, and between HIF-2α and HIF-1β, wherein the interaction is assessed by proximity ligation assay, optionally in MCF-7 cells.

It will be apparent to the skilled person that as the invention provides cyclic peptides, the invention also provides corresponding polynucleotides that comprise or consists of a sequence that encodes the cyclic peptides of the invention. The invention provides a DNA polynucleotide that comprises or consists of a sequence that encodes the cyclic peptide of the invention. The invention also provides an RNA polynucleotide that comprises or consists of a sequence that encodes the cyclic peptide of the invention. The skilled person will appreciate that a polynucleotide, for example a DNA or RNA polynucleotide, may comprise one or more modifications, for example a phosphorothioate modification. The polynucleotide may also comprise one or more other features, for example a promoter, terminator, or a tag for instance, for example the features typical of an expression cassette.

The polynucleotide of the invention may also comprise one or more features that facilitate the cyclisation of the peptide. For example, the polynucleotide may comprise one or more sequences that allows Split-intein circular ligation of peptides and proteins (SICLOPPS) to be performed. For example, the polynucleotide may comprise portions of a split intein which facilitates circularisation of the peptide of the invention.

The invention also provides a nucleic acid vector comprising the nucleic acid of the invention. The skilled person will understand that by nucleic acid vector we include the meaning of a plasmid, artificial chromosome or other nucleic acid structure used to deliver or express the cyclic peptide. The artificial chromosome may be any artificial chromosome and may be selected from, for example, a bacterial artificial chromosome (BAC), a yeast artificial chromosome (YAC), and a Human artificial chromosome (HAC).

The invention also provides a cell that comprises the cyclic peptide of the invention, the polynucleotide of the invention and/or the vector of the invention. The cell of the invention has two main uses, amongst others—i) manufacture of the cyclic peptides or viral vectors comprising the cyclic peptides of the invention, for example; and ii) medical uses for example screening for suitable cyclic peptides for particular situations, or as a therapeutic cell.

In one embodiment then the cell is a cell that is used in the commercial, large scale manufacture of the cyclic peptides of the invention, for example is a bacterial cell such as E. coli, or is a yeast cell such as P. pastoris.

In another embodiment the cell is a cell that either is a direct “diseased” cell, for example taken from a biopsy from a patient. In another embodiment the cell is a cell that is intended to mimic or model a particular disease state. Such cells can be used to screen for appropriate cyclic peptides that are suitable for use in particular therapeutic situations.

Since the cyclic peptides of the invention are able to disrupt the typical response to hypoxia, it will be apparent to the skilled person that the cyclic peptide of the invention, the polynucleotide of the invention and/or the vector of the invention have use in the treatment and/or prevention of diseases, disorders or conditions. For example, in one embodiment the cyclic peptides of the invention are useful in the treatment or prevention of a disease, disorder or condition that experiences a hypoxic environment and requires the typical hypoxia response for maintenance. The cyclic peptides of the invention are also suitable for treatment or prevention of any other disease treatable or preventable by inhibition of dimerization of HIF-1a with HIF1-b and HIF2a with HIF1b and/or inhibits the activity of HIF-1 and HIF-2 and/or HIF-1 or HIF-2 signalling. The cyclic peptides of the invention are also suitable for use in the treatment or prevention of a disease, disorder or condition in which it is desirable to repress hypoxia induced gene expression.

Such diseases, disorders and conditions include Von Hippel-Lindau disease, tumours and cancer.

A tumour is not necessarily the same as cancer. By tumour we include the meaning of any kind of aberrant growth, whether it is benign or malignant. By cancer we include solid cancers and blood cancers. Solid cancers typically refer to an aberrant growth that is or has the potential to be malignant. Blood cancers are not solid cancers and include, for example, lymphomas.

Solid tumours and solid cancers in particular are known to experience a hypoxic tumour microenvironment, and it is known that a hypoxic tumour microenvironment correlates with poor prognosis. Blocking the response to hypoxia using the cyclic peptides of the invention is considered to be useful in the treatment and/or prevention of these diseases, disorders and conditions.

Accordingly, in one embodiment the cell is a human cell. In yet another embodiment the cell is a diseased cell, for example is a cancer cell. In one embodiment the cell is an in vitro cell, such as an in vitro mammalian cell or in vitro human cell. For example, in vitro human cells comprising the peptide, polynucleotide or vector of the invention may be used as part of a screening procedure to determine appropriate treatment strategies. In some embodiments the cell is not an in vivo human cell. In some embodiment the cell is not an in vivo human or animal cell.

In some instances, the isolated polynucleotide of the invention, or vector of the invention may be loaded into a viral vector, for example for therapeutic delivery. The invention therefore also provides a viral vector comprising the polynucleotide or vector of the invention. Viral vectors are well known in the art and examples include but are not limited to: adeno-associated viral vectors (AAV vectors); lentiviral vectors (e.g. those derived from Human Immunodeficiency Virus (HIV)); retroviral vectors (e.g. MMLV). In some embodiments the viral vector is selected from a group comprising a retroviral vector, a lentiviral vector, an adenoviral vector, an adeno-associated viral vector, a bacteriophage vector, and a hybrid viral vector.

In one embodiment the viral vector is not a viral vector that integrates into the genome of the host cell, for example such vectors include AAVs and adenoviral vectors. AAV vectors infect target cells and the delivered genetic material does not integrate into the genome of the host cell. Instead, the delivered genetic material remains episomal. In one embodiment the viral vector is a viral vector that integrates into the genome of the host cell, for example such vectors include the retroviral vectors, for example lentiviral vectors.

As described above, the cyclic peptides of the invention are useful in the treatment or prevention of disease, a disorder or condition. For example, in one embodiment the cyclic peptides of the invention are useful in the treatment or prevention of a disease, disorder or condition:
that experiences a hypoxic environment and requires the typical hypoxia response for maintenance;
that is treatable or preventable by inhibition of dimerization of HIF-1a with HIF1-b and HIF2a with HIF1b and/or inhibits the activity of HIF-1 and HIF-2 and/or HIF-1 or HIF-2 signalling; and/or
in which it is desirable to repress hypoxia induced gene expression.

Accordingly, the invention provides a pharmaceutical composition comprising one or more of the cyclic peptide of the invention, the polynucleotide of the invention, the vector of the invention or the viral vector of the invention.

As used herein, “pharmaceutical composition” means a therapeutically effective formulation for use in the treatment or prevention of diseases, disorders and conditions:
that experience a hypoxic environment and requires the typical hypoxia response for maintenance;
that are treatable or preventable by inhibition of dimerization of HIF-1a with HIF1-b and HIF2a with HIF1b and/or inhibits the activity of HIF-1 and HIF-2 and/or HIF-1 or HIF-2 signalling; and/or
in which it is desirable to repress hypoxia induced gene expression.

Examples of such diseases, disorders and conditions includes cancer, such as solid cancer, or Von Hippel-Lindau disease.

Additional compounds may also be included in the pharmaceutical compositions, such as other peptides, low molecular weight immunomodulating agents, receptor agonists and antagonists, and antimicrobial agents. Other examples include chelating agents such as EDTA, citrate, EGTA or glutathione.

The pharmaceutical compositions may be prepared in a manner known in the art that is sufficiently storage stable and suitable for administration to humans and animals. The pharmaceutical compositions may be lyophilised, e.g. through freeze drying, spray drying, spray cooling, or through use of particle formation from supercritical particle formation.

By “pharmaceutically acceptable” we mean a non-toxic material that does not decrease the effectiveness of the biological activity of the active ingredients, i.e. the cyclic peptides, polynucleotides, vectors or viral vectors of the invention. Such pharmaceutically acceptable buffers, carriers, diluents or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), which are incorporated herein by reference).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the peptide in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the peptide of the composition. The adjuvant may be one or more of colloidal silver, or zinc, copper or silver salts with different anions, for example, but not limited to fluoride, chloride, bromide, iodide, thiocyanate, sulfite, hydroxide, phosphate, carbonate, lactate, glycolate, citrate, borate, tartrate, and acetates of different acyl composition. The adjuvant may also be cationic polymers such as PHMB, cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, sucrose, mannitol, and cyclodextrines, which are added to the composition, e.g., for facilitating lyophilisation. Examples of polymers are starch, cellulose ethers, cellulose, carboxymethylcellulose, hydroxypropylmethyl cellulose, hydroxyethyl cellulose, ethylhydroxyethyl cellulose, ethyl cellulose, methyl cellulose, propyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, poly(lactic acid), poly(glycholic acid) or copolymers thereof with various composition, and polyvinylpyrrolidone, all of different molecular weight, which are added to the composition, e.g. for viscosity control, for achieving bioadhesion, or for protecting the active ingredient (applies to A-C as well) from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The pharmaceutical composition may also contain one or more mono- or di-saccharides such as xylitol, sorbitol, mannitol, lactitiol, isomalt, maltitol or xylosides, and/or monoacylglycerols, such as monolaurin. The characteristics of the carrier are dependent on the route of administration. One route of administration is topical administration. For example, for topical administrations, a preferred carrier is an emulsified cream comprising the active peptide, but other common carriers such as certain petrolatum/mineral-based and vegetable-based ointments can be used, as well as polymer gels, liquid crystalline phases and microemulsions.

It will be appreciated that the pharmaceutical compositions may comprise one or more of the cyclic peptides, polynucleotides, vectors or viral vectors of the invention, for example one, two, three or four different the cyclic peptides, polynucleotides, vectors or viral vectors of the invention. By using a combination of different the cyclic peptides, polynucleotides, vectors or viral vectors of the invention the effect may be increased.

The pharmaceutical compositions of the invention may also be in the form of a liposome, in which the one or more cyclic peptides, polynucleotides, vectors or viral vectors of the invention is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations is can be found in for example U.S. Pat. No. 4,235,871, which is incorporated herein by reference.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microshperes. Preparations of such microspheres can be found in U.S. Pat. No. 5,851,451 and in EP 213 303, which are incorporated herein by reference.

The pharmaceutical compositions of the invention may also be formulated with micellar systems formed by surfactants and block copolymers, preferably those containing poly(ethylene oxide) moieties for prolonging bloodstream circulation time.

The pharmaceutical compositions of the invention may also be in the form of polymer gels, where polymers as such starch, cellulose ethers, cellulose, carboxymethylcellulose, hydroxypropylmethyl cellulose, hydroxyethyl cellulose, ethylhydroxyethyl cellulose, ethyl cellulose, methyl cellulose, propyl cellulose, alginates, chitosan, carageenans, hyaluronic acid and derivatives thereof, polyacrylic acid, polyvinyl imidazole, polysulphonate, polyethylenglycol/polyethylene oxide, polyethylene-oxide/polypropylene oxide copolymers, polyvinylalcohol/polyvinylacetate of different degree of hydrolysis, and polyvinylpyrrolidone are used for thickening of the solution containing the peptide. The polymers may also comprise gelatin or collagen.

Alternatively, the cyclic peptides, polynucleotides, vectors or viral vectors of the invention may be dissolved in saline, water, polyethylene glycol, propylene glycol, ethanol or oils (such as safflower oil, corn oil, peanut oil, cottonseed oil or sesame oil), tragacanth gum, and/or various buffers.

The pharmaceutical composition may also include ions and a defined pH for potentiation of action of anti-microbial polypeptides.

The above compositions of the invention may be subjected to conventional pharmaceutical operations such as sterilisation and/or may contain conventional adjuvants such as preservatives, stabilisers, wetting agents, emulsifiers, buffers, fillers, etc., e.g., as disclosed elsewhere herein.

It will be appreciated by persons skilled in the art that the pharmaceutical compositions of the invention may be administered locally or systemically. Routes of administration include topical (e.g. ophthalmic), ocular, nasal, pulmonary, buccal, parenteral (intravenous, subcutaneous, and intramuscular), oral, vaginal and rectal. Also administration from implants is possible. Suitable preparation forms are, for example granules, powders, tablets, coated tablets, (micro) capsules, suppositories, syrups, emulsions, microemulsions, defined as optically isotropic thermodynamically stable systems consisting of water, oil and surfactant, liquid crystalline phases, defined as systems characterised by long-range order but short-range disorder (examples include lamellar, hexagonal and cubic phases, either water- or oil continuous), or their dispersed counterparts, gels, ointments, dispersions, suspensions, creams, aerosols, droplets or injectable solution in ampoule form and also preparations with protracted release of active compounds, in whose preparation excipients, diluents, adjuvants or carriers are customarily used as described above. The pharmaceutical composition may also be provided in bandages, plasters or in sutures or the like.

In a particular embodiment, the pharmaceutical composition is suitable for oral administration, parenteral administration or topical administration. For example, the pharmaceutical composition may be suitable for topical administration (e.g. ophthalmic administration, in the form of a spray, lotion, paste or drops etc.).

The pharmaceutical compositions will be administered to a patient in a pharmaceutically effective dose. By “pharmaceutically effective dose” is meant a dose that is sufficient to produce the desired effects in relation to the condition for which it is administered. The exact dose is dependent on the, activity of the compound, manner of administration, nature and severity of the disorder, age and body weight of the patient different doses may be needed. The administration of the dose can be carried out both by single administration in the form of an individual dose unit or else several smaller dose units and also by multiple administrations of subdivided doses at specific intervals.

The pharmaceutical compositions of the invention may be administered alone or in combination with other therapeutic agents, such as anti-cancer agents, anti-Von Hippel-Lindau disease agents, antibiotics, anti-inflammatory, immunosuppressive, vasoactive and/or antiseptic agents (such as anti-bacterial agents, anti-fungicides, anti-viral agents, and anti-parasitic agents). Likewise, the pharmaceutical compositions may also contain anti-inflammatory drugs, such as steroids and macrolactam derivatives.

Such additional therapeutic agents may be incorporated as part of the same pharmaceutical composition or may be administered separately.

In addition to the agents such as cyclic peptides, polynucleotides, vectors, viral vectors and pharmaceutical compositions provided by the invention, the invention also provides corresponding uses and methods of use of these agents. In all therapeutic uses and methods of the invention it will be appreciated that the methods and uses may involve the administration of one, or more than one, for example 2, 3, 4, 5, 6, 7, 8, 9 or 10 different cyclic peptides according to the invention, polynucleotides according to the invention, vectors according to the invention, viral vectors according to the invention, cells according to the invention or pharmaceutical compositions of the invention, for example as particular combinations of these agents may have particularly useful therapeutic effects. The uses and methods may also involve the use of different combinations of types of agent, for example may involve the administration of a cyclic peptide of the invention, and a viral vector of the invention, for instance.

For example, the invention provides one or more of the cyclic peptides according to the invention, the polynucleotides according to the invention, the vectors according to the invention, the viral vectors according to the invention, the cells according to the invention or the pharmaceutical compositions of the invention or combination thereof, for use in medicine, for example for use in the treatment of prevention of disease a disorder or a condition.

The invention also provides one or more of the cyclic peptides according to the invention, the polynucleotides according to the invention, the vectors according to the invention, the viral vectors according to the inventions, the cells according to the invention or the pharmaceutical compositions of the invention or combination thereof, for use in the treatment or prevention of cancer. The cancer may be a solid cancer or may be a non-solid cancer, for example a blood cancer.

Preferably the cancer is a cancer:
that experiences a hypoxic environment and requires the typical hypoxia response for maintenance;
that is treatable or preventable by inhibition of dimerization of HIF-1a with HIF1-b and HIF2a with HIF1b and/or inhibits the activity of HIF-1 and HIF-2 and/or HIF-1 or HIF-2 signalling; and/or
in which it is desirable to repress hypoxia induced gene expression.

Preferably the cancer is a solid cancer.

In some embodiments, the cancer is selected from the group comprising or consisting of:
acute lymphoblastic leukemia (ALL), Acute myeloid leukemia, Adrenocortical carcinoma, AIDS-related cancers, AIDS-related lymphoma, Anal cancer, Appendix cancer, Astrocytoma, childhood cerebellar or cerebral, Basal-cell carcinoma, Bile duct cancer, extrahepatic (see cholangiocarcinoma), Bladder cancer, Bone tumor, osteosarcoma/malignant fibrous histiocytoma, Brainstem glioma, Brain cancer, Brain tumor, cerebellar astrocytoma, Brain tumor, cerebral astrocytoma/malignant glioma, Brain tumor, ependymoma, Brain tumor, medulloblastoma, Brain tumor, supratentorial primitive neuroectodermal tumors, Breast cancer, Bronchial adenomas/carcinoids, Burkitt's lymphoma, Carcinoid tumor, childhood, Carcinoid tumor, gastrointestinal, Carcinoma of unknown primary, Cerebellar astrocytoma, Cerebral astrocytoma/malignant glioma, Cervical cancer, Chondrosarcoma, Chronic lymphocytic leukemia, Chronic myelogenous leukemia, Chronic myeloproliferative disorders, Colon cancer, Cutaneous T-cell lymphoma, Desmoplastic small round cell tumor, Endometrial cancer, Ependymoma, Esophageal cancer, Ewing's sarcoma, Extracranial germ cell tumor, Extragonadal germ cell tumor, Extrahepatic bile duct cancer, intraocular melanoma, retinoblastoma, Gallbladder cancer, Gastric (stomach) cancer, Gastrointestinal carcinoid tumor, Gastrointestinal stromal tumor (GIST), Germ cell tumor: extracranial, extragonadal, or ovarian, Gestational trophoblastic tumor, Glioma of the brain stem, Glioma, childhood cerebral astrocytoma, Glioma, childhood visual pathway and hypothalamic, Gastric carcinoid, Hairy cell leukemia, Hepatocellular (liver) cancer, Hodgkin lymphoma, Hypopharyngeal cancer, Hypothalamic and visual pathway glioma, childhood, Intraocular melanoma, Islet cell carcinoma (endocrine pancreas), Kaposi sarcoma, Kidney cancer (renal cell cancer), Laryngeal cancer, Leukaemias, Leukaemia, acute lymphoblastic (also called acute lymphocytic leukaemia), Leukaemia, acute myeloid (also called acute myelogenous leukemia), Leukaemia, chronic lymphocytic (also called chronic lymphocytic leukemia), Leukemia, chronic myelogenous (also called chronic myeloid leukemia), Leukemia, hairy cell, Lip and oral cavity cancer, Liposarcoma, Liver cancer (primary), Lung cancer, non-small cell, Lung cancer, small cell, Lymphomas, Lymphoma, AIDS-related, Lymphoma, Burkitt, Lymphoma, cutaneous T-Cell, Lymphoma, Hodgkin, Lymphomas, Non-Hodgkin, Lymphoma, primary central nervous system, Macroglobulinemia, Waldenstrom, Malignant fibrous histiocytoma of bone/osteosarcoma, Medulloblastoma, Melanoma, Merkel cell cancer, Mesothelioma, Mouth cancer, Multiple endocrine neoplasia syndrome, Multiple myeloma/plasma cell neoplasm, Mycosis fungoides, Myelodysplastic syndromes Myelodysplastic/myeloproliferative diseases, Myelogenous leukemia, chronic Myeloid leukemia, adult acute, Myeloid leukemia, childhood acute, Myeloma, multiple (cancer of the bone-marrow), Myeloproliferative disorders, chronic, Myxoma, Nasal cavity and paranasal sinus cancer, Nasopharyngeal carcinoma, Neuroblastoma, Non-Hodgkin lymphoma, Non-small cell lung cancer, Oligodendroglioma, Oral cancer, Oropharyngeal cancer, Osteosarcoma/malignant fibrous histiocytoma of bone, Ovarian cancer, Ovarian epithelial cancer (surface epithelial-stromal tumor), Ovarian germ cell tumor, Ovarian low malignant potential tumor, Pancreatic cancer, Pancreatic cancer, islet cell, Paranasal sinus and nasal cavity cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, childhood, Pituitary adenoma, Plasma cell neoplasia/Multiple myeloma, Pleuropulmonary blastoma, Primary central nervous system lymphoma, Prostate cancer, Rectal cancer, Renal cell carcinoma, Renal pelvis and ureter, transitional cell cancer, Rhabdomyosarcoma, childhood, Salivary gland cancer, Sarcoma, Ewing family of tumors, Sarcoma, Kaposi, Sarcoma, soft tissue, Sarcoma, uterine, Sezary syndrome, Skin cancer (non-melanoma), Skin cancer (melanoma), Skin carcinoma, Merkel cell, Small cell lung cancer, Small intestine cancer, Soft tissue sarcoma, Squamous cell carcinoma—see skin cancer (non-melanoma), Squamous neck cancer with occult primary, metastatic, Stomach cancer, Supratentorial primitive neuroectodermal tumor, childhood, T-Cell lymphoma, cutaneous, Testicular cancer, Throat cancer, Thymoma, Thymoma and thymic carcinoma, Thyroid cancer, Thyroid cancer, Transitional cell cancer of the renal pelvis and ureter, Trophoblastic tumor, gestational, Unknown primary site, Ureter and renal pelvis, transitional cell cancer, Urethral cancer, Uterine cancer, endometrial, Uterine sarcoma, Vaginal cancer, Visual pathway and hypothalamic glioma, Vulvar cancer, Waldenstrom macroglobulinemia and/or Wilms tumor (kidney cancer).

The invention also provides one or more of the cyclic peptides according to the invention, the polynucleotides according to the invention, the vectors according to the invention, the viral vectors according to the invention, the cells according to the invention or the pharmaceutical compositions of the invention or combination thereof, for use in the treatment or prevention of a disease, disorder or condition:
that experiences a hypoxic environment and requires the typical hypoxia response for maintenance;
that is treatable or preventable by inhibition of dimerization of HIF-1a with HIF1-b and HIF2a with HIF1b and/or inhibits the activity of HIF-1 and HIF-2 and/or HIF-1 or HIF-2 signalling; and/or
in which it is desirable to repress hypoxia induced gene expression.

It will be appreciated that any of the therapeutic agents described herein, for example the cyclic peptide according to the invention, the polynucleotide according to the invention, the vector according to the invention, the viral vector according to the invention, the cell according to the invention or the pharmaceutical composition of the invention, can be formulated as a composition. For example any of the cyclic peptide according to the invention, the polynucleotide according to the invention, the vector according to the invention, the viral vector according to the invention, or the cell according to the invention can be formulated as a pharmaceutical composition. It will also be clear that any of the cyclic peptide according to the invention, the polynucleotide according to the invention, the vector according to the invention, the viral vector according to the invention, the cell according to the invention or the pharmaceutical composition of the invention can be formulated with one or more further therapeutic agents, for example one or more further anti-cancer therapeutic agents or one or more further agents for the treatment of Von Hippel-Lindau disease.

It will also be clear that any one or more of the cyclic peptides according to the invention, the polynucleotides according to the invention, the vectors according to the invention, the viral vectors according to the invention, the cells according to the invention or the pharmaceuticals composition of the invention or combination thereof, can be administered as part of a combination therapy. For example, the cyclic peptides according to the invention, the polynucleotides according to the invention, the vectors according to the invention, the viral vectors according to the invention, the cells according to the invention or the pharmaceutical compositions of the invention or combination thereof, can be administered prior to a further therapeutic agent, for example prior to the administration of one or more further anti-cancer therapeutic agents or one or more further agents for the treatment of von Hippel-Lindau disease.

It will also be clear that any one or more of the cyclic peptides according to the invention, the polynucleotides according to the invention, the vectors according to the invention, the viral vectors according to the invention, the cells according to the invention or the pharmaceutical compositions of the invention or combination thereof, can be administered following the administration of a further therapeutic agent, for example following the administration of one or more further anti-cancer therapeutic agents or one or more further agents for the treatment of von Hippel-Lindau disease.

It will also be clear that any one or more of the cyclic peptides according to the invention, the polynucleotides according to the invention, the vectors according to the invention, the viral vectors according to the invention, the cells according to the invention or the pharmaceutical compositions of the invention or combination thereof, can be administered simultaneously to the administration of a further therapeutic agent, for example simultaneous to the administration of one or more further anti-cancer therapeutic agents or one or more further agents for the treatment of von Hippel-Lindau disease. The simultaneous administration may involve the administration of a single composition comprising both the cyclic peptide according to the invention, the polynucleotide according to the invention, the vector according to the invention, the viral vector according to the invention, the cell according to the invention or the pharmaceutical composition of the invention or combination thereof, and the one or more further therapeutic agents, for example one or more further anti-cancer therapeutic agents or one or more further agents for the treatment of von Hippel-Lindau disease. The simultaneous administration may instead involve the administration of separate compositions, a first composition comprising the cyclic peptide according to the invention, the polynucleotide according to the invention, the vector according to the invention, the viral vector according to the invention, the cell according to the invention or the pharmaceutical composition of the invention and a second composition comprising the one or more further therapeutic agents, for example one or more further anti-cancer therapeutic agents or one or more further agents for the treatment of von Hippel-Lindau disease.

The invention also provides a method for the treatment or prevention of a disease, disorder or condition, wherein the method comprises administration of one or more of the cyclic peptides according to the invention, the polynucleotides according to the invention, the vectors according to the invention, the viral vectors according to the invention, the cells according to the invention or the pharmaceutical compositions of the invention or combination thereof.

Preferences for the disease, peptide and other features are described elsewhere herein. For example, by disease, disorder or condition we include the meaning of any disease, disorder or condition:
that experiences a hypoxic environment and requires the typical hypoxia response for maintenance;
that is treatable or preventable by inhibition of dimerization of HIF-1a with HIF1-b and HIF2a with HIF1b and/or inhibits the activity of HIF-1 and HIF-2 and/or HIF-1 or HIF-2 signalling; and/or
in which it is desirable to repress hypoxia induced gene expression.

Examples of such diseases, disorders and conditions includes tumours, cancers including solid cancers and blood cancers, and von Hippel-Lindau disease.

Accordingly, the invention provides a method for the treatment or prevention of a disease, disorder or condition:
that experiences a hypoxic environment and requires the typical hypoxia response for maintenance;
that is treatable or preventable by inhibition of dimerization of HIF-1a with HIF1-b and HIF2a with HIF1b and/or inhibits the activity of HIF-1 and HIF-2 and/or HIF-1 or HIF-2 signalling; and/or
in which it is desirable to repress hypoxia induced gene expression;
   wherein the method comprises administration of one or more of the cyclic peptides according to the invention, the polynucleotides according to the invention, the vectors according to the invention, the viral vectors according to the invention, the cells according to the invention or the pharmaceutical compositions of the invention.

The invention also provides a method for the treatment or prevention of cancer or a tumour, for example a solid cancer or solid tumour, wherein the method comprises administration of one or more of the cyclic peptides according to the invention, the polynucleotides according to the invention, the vectors according to the invention, the viral vectors according to the invention, the cells according to the invention or the pharmaceutical compositions of the invention or combination thereof.

The invention also provides:
use of one or more of the cyclic peptides according to the invention, the polynucleotides according to the invention, the vectors according to the invention, the viral vectors according to the invention, the cells according to the invention or the pharmaceutical compositions of the invention or combination thereof, in a method of manufacture of a medicament for use in medicine;
use of one or more of the cyclic peptides according to the invention, the polynucleotides according to the invention, the vectors according to the invention, the viral vectors according to the invention, the cells according to the invention or the pharmaceutical compositions of the invention or combination thereof, in a method of manufacture of a medicament for use in the treatment or prevention of a disease, disorder or condition:
   that experiences a hypoxic environment and requires the typical hypoxia response for maintenance;
   that is treatable or preventable by inhibition of dimerization of HIF-1a with HIF1-b and HIF2a with HIF1b and/or inhibits the activity of HIF-1 and HIF-2 and/or HIF-1 or HIF-2 signalling; and/or
   in which it is desirable to repress hypoxia induced gene expression; and
use of one or more of the cyclic peptides according to the invention, the polynucleotides according to the invention, the vectors according to the invention, the viral vectors according to the invention, the cells according to the invention or the pharmaceutical compositions of the invention or combination thereof, in a method of manufacture of a medicament for use in for use in the treatment or prevention of cancer, for example a solid cancer or tumour.

The skilled person will also understand that various components and agents of the present invention lend themselves to being provided as part of a kit, or kit of parts. For example in one embodiment the invention provides a kit comprising one or more of:
a cyclic peptide according to the invention;
a polynucleotide according to the invention;
a nucleic acid vector according to the invention;
a cell according to the invention;
a viral vector according to the invention; or
a pharmaceutical composition according to the invention.

The kit may comprise any number of these agents, for example the kit may comprise 1, 2, 3, 4, 5, 6, 7, 8, 9, 10 or more than 10 different cyclic peptides of the invention; and/or may comprise 1, 2, 3, 4, 5, 6, 7, 8, 9, 10 or more than 10 polynucleotides of the invention; for example may comprise 1, 2, 3, 4, 5, 6, 7, 8, 9, 10 or more than 10 different nucleic acid vectors of the invention; for example may comprise 1, 2, 3, 4, 5, 6, 7, 8, 9, 10 or more than 10 different cells of the invention; for example may comprise 1, 2, 3, 4, 5, 6, 7, 8, 9, 10 or more than 10 different viral vectors of the invention; for example may comprise 1, 2, 3, 4, 5, 6, 7, 8, 9, 10 or more than 10 different pharmaceutical compositions of the invention.

For example, in one embodiment the kit is suitable for determining the most appropriate cyclic peptide therapy to treat a particular tumour.

The kit may also comprise appropriate control cells and reagents.

The listing or discussion of an apparently prior-published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention. To exemplify how the preferences and options described throughout can be combined, the invention provides:
a cyclic peptide of sequence cys X₁ leu X₂ X₃ X₄ [Formula 1] [SEQ ID NO: 1] wherein the cyclic peptide is capable of binding to HIF-1 and HIF-2 with a similar affinity and wherein the cyclic peptide prevents or reduces the hypoxia induced expression from a promoter that comprises one or more hypoxia-responsive elements under hypoxic conditions;
a viral vector comprising a nucleic acid that encodes the cyclic peptide of sequence cys X₁ leu X₂ X₃ X₄ [Formula 1] [SEQ ID NO: 1]; and
a method of treating cancer wherein the method comprises administering a pharmaceutical composition, wherein the pharmaceutical composition comprises a cyclic peptide of sequence cys X₁ leu X₂ X₃ X₄ [Formula 1] [SEQ ID NO: 1], and a viral vector comprising a polynucleotide that encodes the cyclic peptide of sequence cys X₁ leu X₂ X₃ X₄ [Formula 1] [SEQ ID NO: 1];
and wherein the cyclic peptide disrupts the interaction between HIF-1a and HIF-1b, optionally wherein the interaction is assessed by proximity ligation assay, optionally in MCF-7 cells.

### FIGURE LEGENDS

FIG. 1

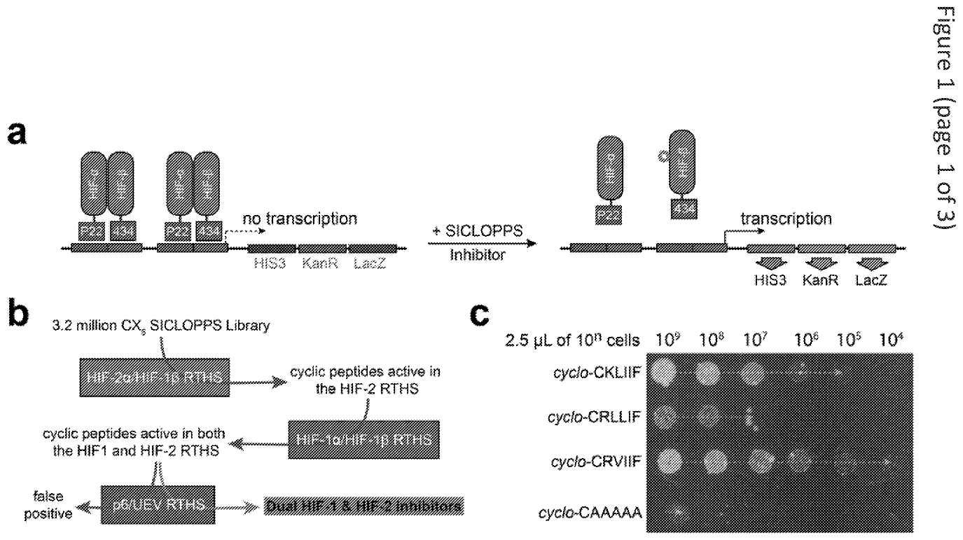

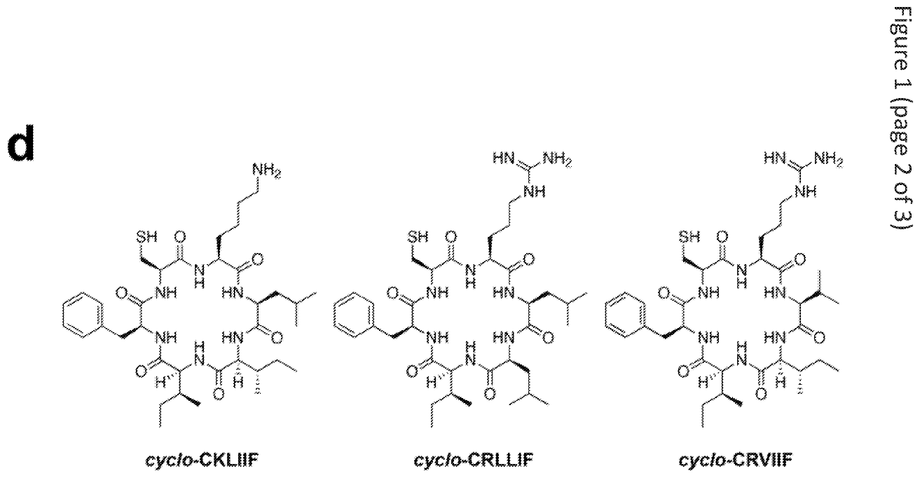

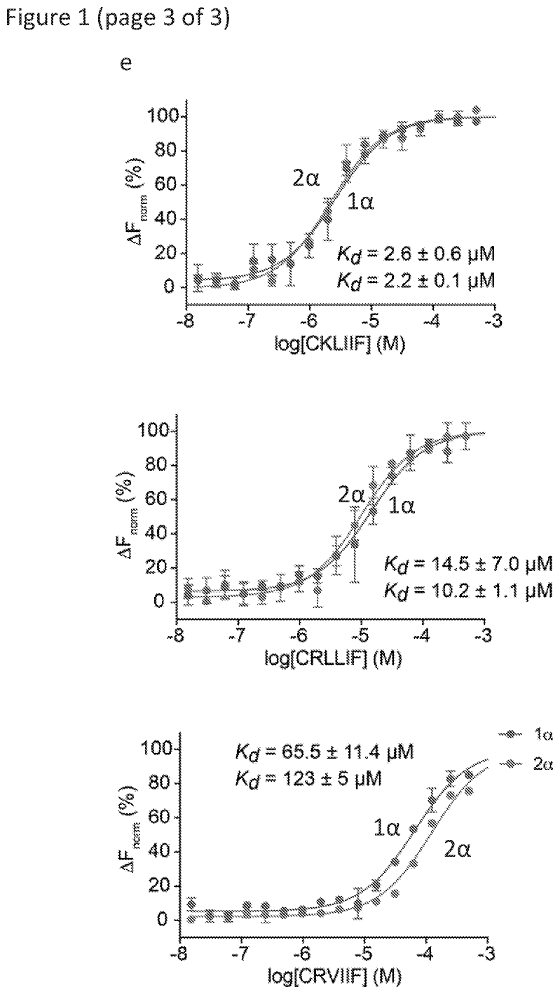

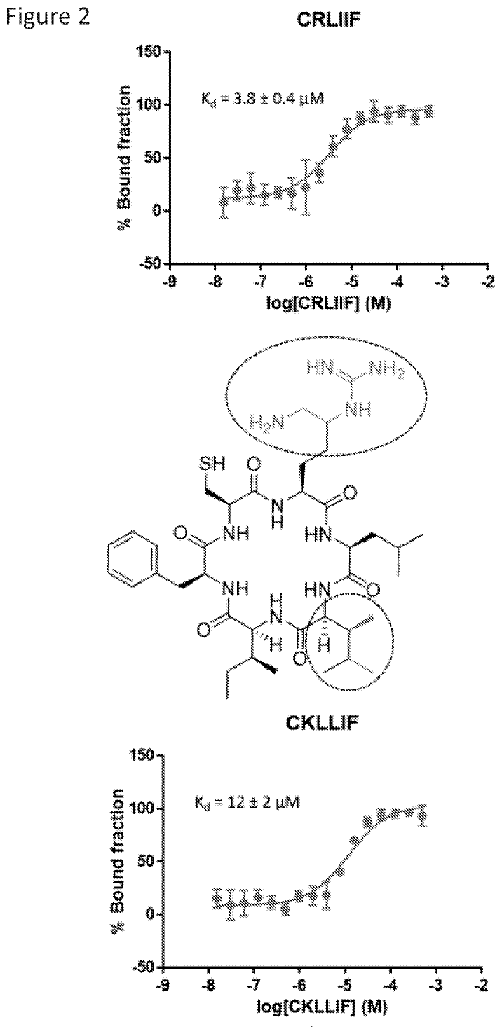

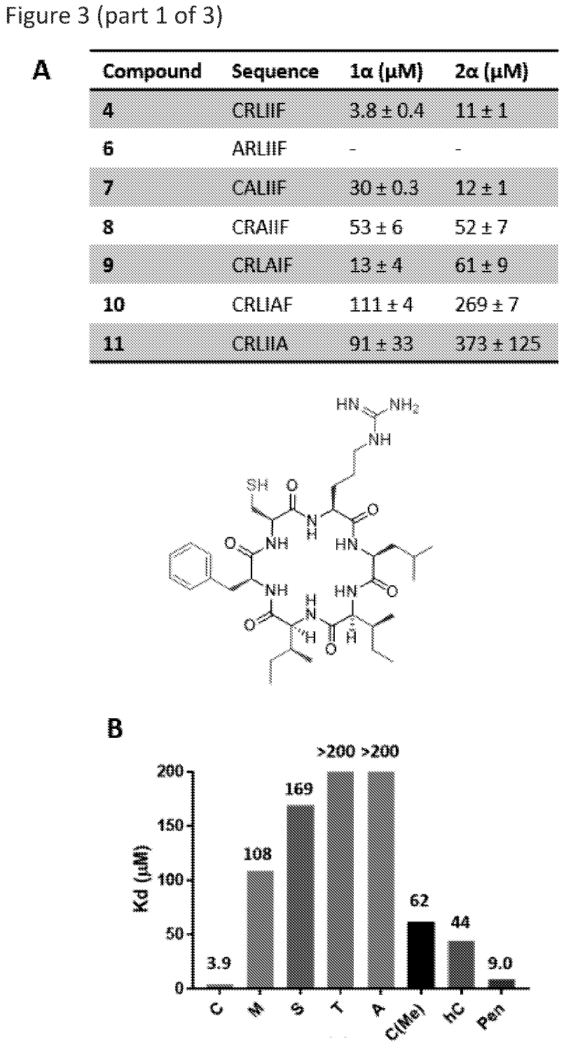

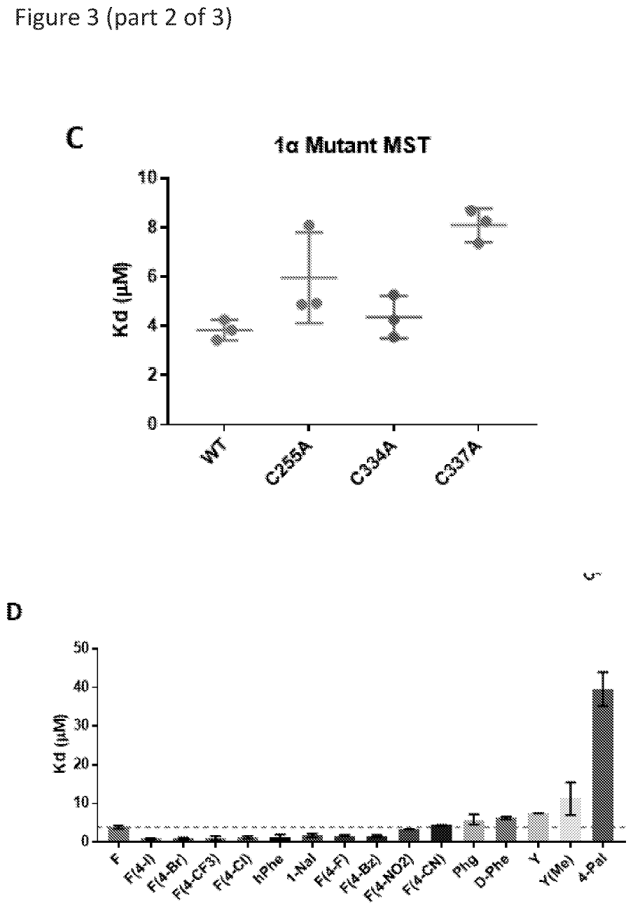

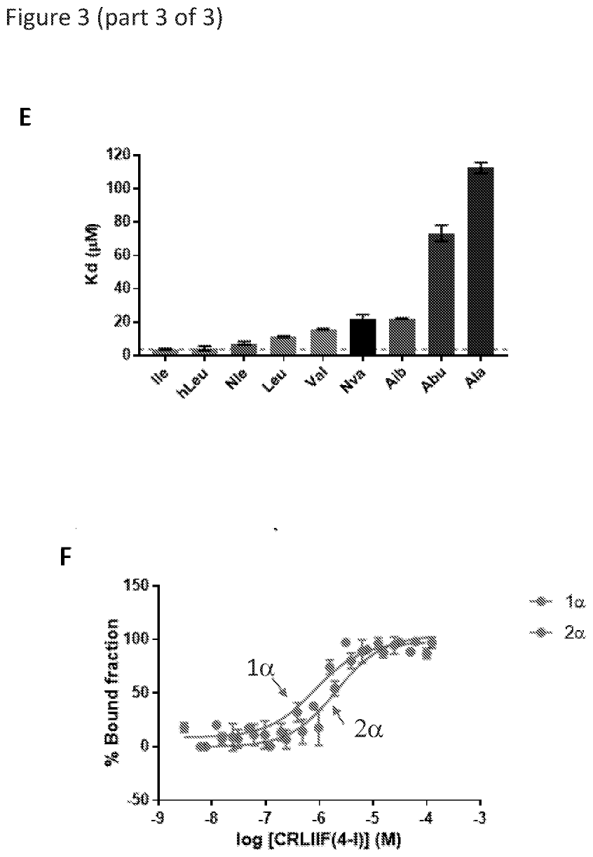

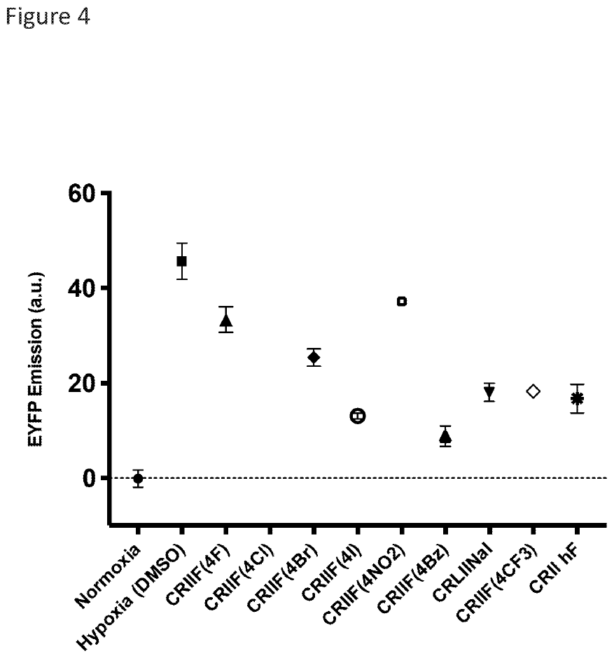

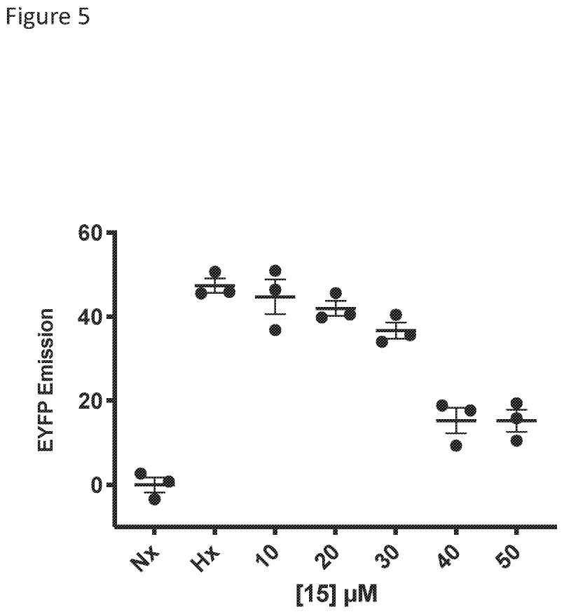

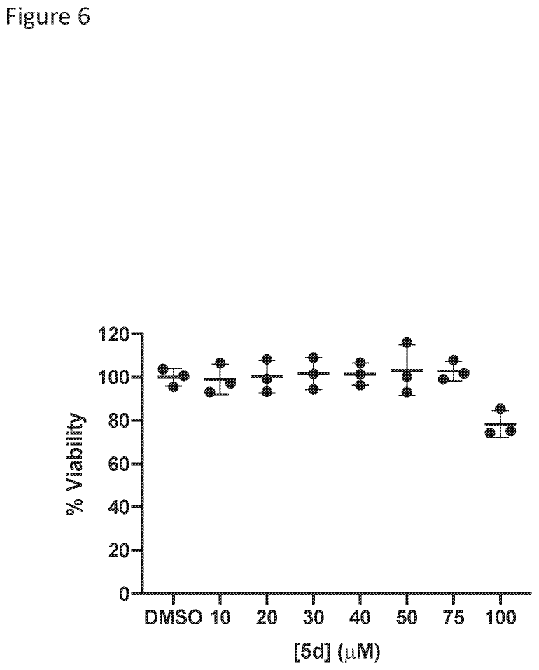

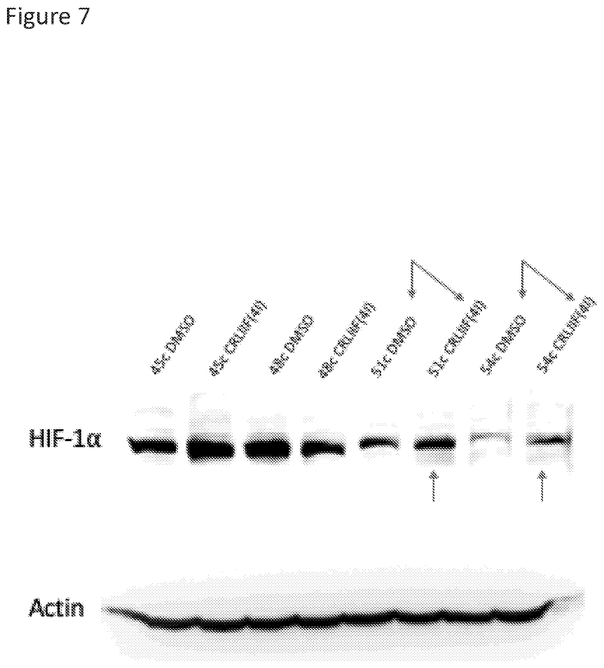

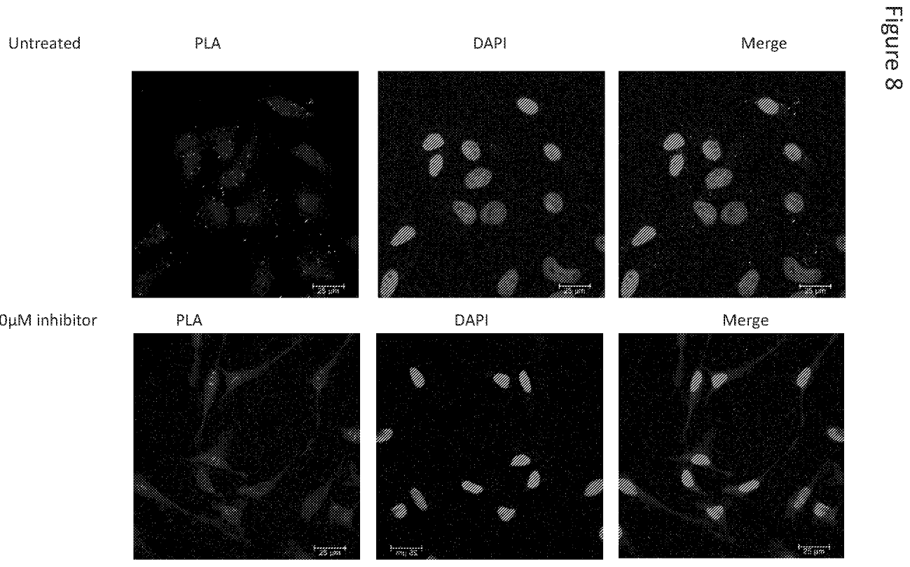

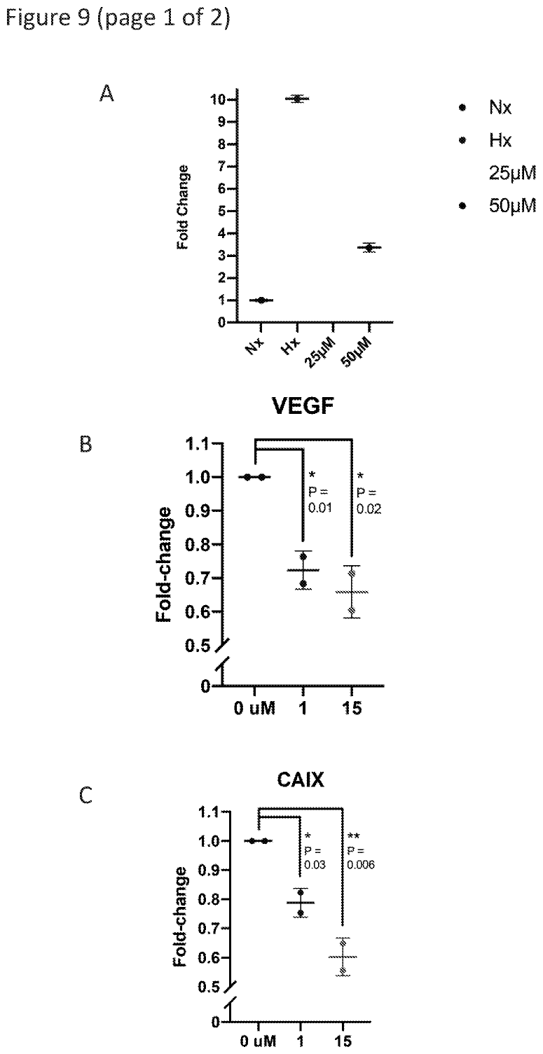

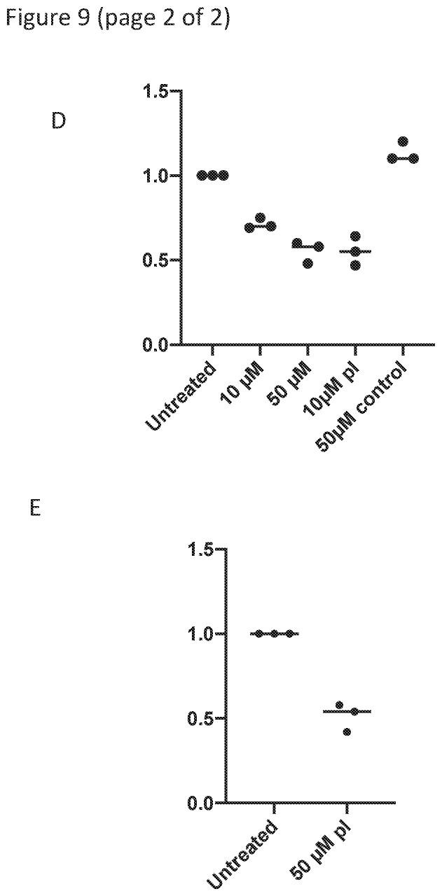

A screen for cyclic peptide inhibitors of HIF-2 dimerization was performed using SICLOPPS with a HIF-2 RTHS, using a method described previously.^{8,9} (FIG. 1a). A secondary screen was then also conducted on the HIF-1 RTHS to compare selectivity, after which the hits found to inhibit both HIF-2 and HIF-1 were chosen for further review (FIG. 1b). SICLOPPS plasmids that produced a growth advantage in both systems were further screened in an unrelated p6/UEV RTHS to eliminate false positives (FIG. 1b). From the top five inhibitors, three peptides—CKLIIF, CRLLIF and CRVIIF were selected as they possessed high sequence homology (FIGS. 1c and 1d).

FIG. 2

The intermediary peptides CRLIIF and CKLLIF, prepared by swapping one of the residues at the 2 or 4 position respectively, were synthesised and tested via MST against 1α PAS-B

FIG. 3

Alanine scanning to determine the residues critical for binding activity in CRLIIF (FIG. 3a). Affinity of various substitutions at C1, I5 and F6 positions (FIG. 3b). Three 1α PAS-B mutants were prepared with mutations C255A, C334A or C337A and the activity of the peptide CRLIIF again tested by MST (FIG. 3c). Peptide derivatives containing unnatural amino acid substitutions at the F6 position were synthesized and their affinity investigated against the HIF-1α PAS-B domain (FIG. 3d). Peptide derivatives containing amino acid substitutions at the I5 position were synthesized and their affinity investigated against the HIF-1α PAS-B domain (FIG. 3e). The affinity of CRLIIF(4I) for HIF-2α PAS-B was also tested (FIG. 3f).

FIG. 4

The effect of the cyclic peptides on cellular systems was assessed. A cell-based assay was developed in which the expression of yellow fluorescent protein (YFP) was put under the control of the HRE. A cassette was designed containing the YFP gene preceded by three copies of the HRE sequence. This cassette in turn was stably integrated into the chromosome of T-REX-293 cell line as described previously. The 9 most potent molecules were tested in YFP-reporter HEK cell line (FIG. 4). In these cells, hypoxia results in increased expression of YFP (directly by HIF), so a HIF inhibitor would be expected to reduce the fluorescence of these cells when incubated in hypoxia.

FIG. 5

The 4-iodo derivative was tested at various doses in the YFP reporter assay and showed an IC₅₀ in the above YFP assay of around 35 μM.

FIG. 6

The 4-iodo derivative was tested in a commercial cell-viability assay, and was not toxic to cells at up to twice the tested doses.

FIG. 7

The binding of 4-iodo derivative to HIF-1a was assessed by CETSA in MCF-7 cells. Stabilisation of HIF-1a by this compound can be observed at 51° C. and 54° C.

FIG. 8

The ability of the 4-iodo derivative to disrupt the interaction of HIF-1a and HIF-1b was assessed by proximity ligation assay (PLA) in MCF-7 cells.

FIG. 9

A) The effect of the 4-iodo derivative (25 μM and 50 μM) on the expression of angiopoietin in PANC-1 cells. B) The effect of 4-iodo derivative (10 μM) and its parent CRLIIF (#1) on VEGF expression in MCF-7 cells. C) The effect of 4-iodo derivative (10 μM) and its parent CRLIIF (#1) on CAIX expression in MCF-7 cells. D) The effect of para-iodo derivative (10 μM, labelled pI on plot) and its parent peptide (10 μM and 50 μM), and a negative control cyclic peptide (single amino acid change from cyclic peptide #15 at 50 μM) on CAIX expression in MCF-7 cells. E) The effect of the para-iodo derivative (50 μM, labelled pI on plot) on CAIX expression in Hela cells.

The invention will now be exemplified by the following non-limiting examples.

Insofar as cyclic peptide #s [1], [2], [3], [6], [7], [8], [9], [10], [11], [12a], [12b], [12c], [12d], [13a], [13b], [13c], [13d], [14], [15], [16], [17], [30], [35], and [38] described below are included in the specification and the Examples, they are included for comparative purposes only.

For the avoidance of doubt, it is intended that cyclic peptide #s [4], [5], [18], [19], [20], [21], [22], [23], [24], [25], [26], [27], [28], [29], [31], [32], [33], [34], [36], [39] and [40] described below are intended to fall within the scope of the claims.

In one embodiment the invention provides cyclic peptide #s [4], [5], [18], [19], [20], [21], [22], [23], [24], [25], [26], [27], [28], [29], [31], [32], [33], [34], [36] and [39], and with [40] the structure as defined below.

In one embodiment the cyclic peptide of the invention is not any one or more or all of cyclic peptide #s [1], [2], [3], [6], [7], [8], [9], [10], [11], [12a], [12b], [12c], [12d], [13a], [13b], [13c], [13d], [14], [15], [16], [17], [30], [35], [37] and [38].

The table below shows concordance between the sequence identifiers used herein, and the number assigned to each individual cyclic peptide described in Example 4:

| | | | Cyclic peptide |
|---|---|---|---|
| SEQ ID NO: | Sequence | Notes | # |
| SEQ ID NO: 1 | cys X₁ leu X₂ X₃ X₄ | Consensus sequence | |
| SEQ ID NO: 2 | cys arg leu ile ile X₄ | Consensus sequence | |
| SEQ ID NO: 3 | cys arg leu leu ile X₄ | Consensus sequence | |
| SEQ ID NO: 4 | cys lys leu ile ile X₄ | Consensus sequence | |
| SEQ ID NO: 5 | cys lys leu leu ile X₄ | Consensus sequence | |
| SEQ ID NO: 6 | cys arg leu ile ile | | 4 |
| | phe | | |
| SEQ ID NO: 7 | cys lys leu leu ile | | 5 |
| | phe | | |
| SEQ ID NO: 8 | cys arg leu ile ile | | |
| | phe | | |
| SEQ ID NO: 9 | cys arg leu ile ile | | |
| | phe(4-Br) | | |
| SEQ ID NO: | cys arg leu ile ile | | |
| 10 | phe(4-CF₃) | | |
| SEQ ID NO: | cys arg leu ile ile | | |
| 11 | phe(4-Cl) | | |
| SEQ ID NO: | cys arg leu ile ile | | |
| 12 | (h-phe) | | |
| SEQ ID NO: | cys arg leu ile ile | | |
| 13 | (ala(1-naph)) | | |
| SEQ ID NO: | cys arg leu ile ile | | |
| 14 | phe(4-F) | | |
| SEQ ID NO: | cys arg leu ile ile | | |
| 15 | phe(4-Bz) | | |
| SEQ ID NO: | cys arg leu ile ile | | |
| 16 | phe(4-NO2) | | |
| SEQ ID NO: | cys arg leu ile ile | | |
| 17 | phe(4-CN) | | |
| SEQ ID NO: | cys arg leu ile | | |
| 18 | ile(gly(Ph)) | | |
| SEQ ID NO: | cys arg leu ile ile | | |
| 19 | (D-phe) | | |
| SEQ ID NO: | cys arg leu ile ile tyr | | |
| 20 | | | |
| SEQ ID NO: | | BLANK | |
| 21 | | | |
| SEQ ID NO: | | BLANK | |
| 22 | | | |
| SEQ ID NO: | cys arg leu ile leu | | |
| 23 | phe | | |
| SEQ ID NO: | | BLANK | |
| 24 | | | |
| SEQ ID NO: | cys lys leu iso iso | Included for comparative | 1 |
| 25 | phe | purposes only | |
| SEQ ID NO: | cys arg leu leu iso | Included for comparative | 2 |
| 26 | phe | purposes only | |
| SEQ ID NO: | cys arg val iso iso | Included for comparative | 3 |
| 27 | phe | purposes only | |
| SEQ ID NO: | Cys leu leu phe val | | |
| 28 | tyr | | |
| SEQ ID NO: | Cys ala ala ala ala | | |
| 29 | ala | | |
| SEQ ID NO: | Ala arg leu iso iso | Included for comparative | 9 |
| 30 | phe | purposes only | |
| SEQ ID NO: | Cys ala leu iso iso | Included for comparative | 10 |
| 31 | phe | purposes only | |
| SEQ ID NO: | Cys arg ala iso iso | Included for comparative | 11 |
| 32 | phe | purposes only | |
| SEQ ID NO: | Cys arg leu ala iso | Included for comparative | 6 |
| 33 | phe | purposes only | |
| SEQ ID NO: | Cys arg leu iso ala | Included for comparative | 7 |
| 34 | phe | purposes only | |
| SEQ ID NO: | Cys arg leu iso iso | Included for comparative | 8 |
| 35 | ala | purposes only | |
| SEQ ID NO: | H₂N-F(Pen)RLII-OH | Included for comparative | 12a |
| 36 | | purposes only | |
| SEQ ID NO: | H₂N-IIF(Pen(SPy))RL- | Included for comparative | 12b |
| 37 | OH | purposes only | |
| SEQ ID NO: | Cyclo-Pen(SPy)RLIIF | Included for comparative | 12c |
| 38 | | purposes only | |
| SEQ ID NO: | Cyclo-PenRLIIF | Included for comparative | 12d |
| 39 | | purposes only | |
| SEQ ID NO: | H₂N-F(hC)RLII-OH | Included for comparative | 13a |
| 40 | | purposes only | |
| SEQ ID NO: | H₂N-IIF(hC(SPy)RL-OH | Included for comparative | 13b |
| 41 | | purposes only | |
| SEQ ID NO: | Cyclo-hC(SPy)RLIIF | Included for comparative | 13c |
| 42 | | purposes only | |
| SEQ ID NO: | Cyclo-hCRLIIF | Included for comparative | 13d |
| 43 | | purposes only | |
| SEQ ID NO: | Cyclo-MRLIIF | Included for comparative | 14 |
| 44 | | purposes only | |
| SEQ ID NO: | Cyclo-SRLIIF | Included for comparative | 15 |
| 45 | | purposes only | |
| SEQ ID NO: | Cyclo-TRLIIF | Included for comparative | 16 |
| 46 | | purposes only | |
| SEQ ID NO: | Cyclo-C(Me)RLIIF | Included for comparative | 17 |
| 47 | | purposes only | |
| SEQ ID NO: | Cyclo-CRLIIF(4-I) | | 18 |
| 48 | | | |
| SEQ ID NO: | Cyclo-CRLIIF(4-Br) | | 19 |
| 49 | | | |
| SEQ ID NO: | Cyclo-CRLIIF(4-CF₃) | | 20 |
| 50 | | | |
| SEQ ID NO: | Cyclo-CRLIIF(4-Cl) | | 21 |
| 51 | | | |
| SEQ ID NO: | Cyclo-CRLII(hPhe) | | 22 |
| 52 | | | |
| SEQ ID NO: | Cyclo-CRLII(1-Nal) | | 23 |
| 53 | | | |
| SEQ ID NO: | Cyclo-CRLIIF(4-F) | | 24 |
| 54 | | | |
| SEQ ID NO: | Cyclo-CRLIIF(4-Bz) | | 25 |
| 55 | | | |
| SEQ ID NO: | Cyclo-CRLIIF(4-NO₂) | | 26 |
| 56 | | | |
| SEQ ID NO: | Cyclo-CRLIIF(4-CN) | | 27 |
| 57 | | | |
| SEQ ID NO: | Cyclo-CRLII(Phg) | | 28 |
| 58 | | | |
| SEQ ID NO: | Cyclo-CRLII(D-Phe) | | 32 |
| 59 | | | |
| SEQ ID NO: | Cyclo-CRLIIY | | 29 |
| 60 | | | |
| SEQ ID NO: | Cyclo-CRLIIY(Me) | Included for comparative | 30 |
| 61 | | purposes only | |
| SEQ ID NO: | Cyclo-CRLII(4-Pal) | | 31 |
| 62 | | | |
| SEQ ID NO: | Cyclo-CRLI(hL)F | | 33 |
| 63 | | | |
| SEQ ID NO: | Cyclo-CRLI(Nle)F | | 34 |
| 64 | | | |
| SEQ ID NO: | Cyclo-CRLILF | | 36 |
| 65 | | | |
| SEQ ID NO: | Cyclo-CRLI(Nva)F | Included for comparative | 35 |
| 66 | | purposes only | |
| SEQ ID NO: | Cyclo-CRLIVF | Included for comparative | 37 |
| 67 | | purposes only | |
| SEQ ID NO: | Cyclo-CRLI(Aib)F | Included for comparative | 38 |
| 68 | | purposes only | |
| SEQ ID NO: | Cyclo-CRLI(Abu)F | | 39 |
| 69 | | | |
| SEQ ID NO: | CXXXXX | | |
| 70 | | | |
| SEQ ID NO: | cyclo-cys arg leu ile | | 40 |
| 71 | h-leu (4-I)phe | | |

### EXAMPLES

A screen for cyclic peptide inhibitors of HIF-2 dimerization was performed using SICLOPPS with a HIF-2 RTHS, using a method described previously.^{8,9} (FIG. 1a). A secondary screen was then also conducted on the HIF-1 RTHS to compare selectivity, after which the hits found to inhibit both HIF-2 and HIF-1 were chosen for further review (FIG. 1b). SICLOPPS plasmids that produced a growth advantage in both systems were further screened in an unrelated p6/UEV RTHS to eliminate false positives (FIG. 1b).

From the top five inhibitors, three peptides—CKLIIF [SEQ ID NO: 25], CRLLIF [SEQ ID NO: 26] and CRVIIF [SEQ ID NO: 27] were selected as they possessed high sequence homology (FIGS. 1c and 1d). It was expected that the similarity between these peptides would translate to a common structure-affinity relationship trend between the three, and that any differences in affinity could be used to inform optimisation efforts. In addition, all three peptides contained a basic side chain (arginine or lysine), which are protonated under physiological conditions, and would therefore aid solubility and potentially provide a useful polar interaction with the proteins themselves. This addresses a key problematic issue with the previously reported HIF-1 inhibitor, CLLFVY [SEQ ID NO: 28] which was comparatively insoluble. Secondary screening of these peptides confirmed that all three peptides could effect a greater regrowth response than the control CAAAAA [SEQ ID NO: 29] (FIG. 1c). Whilst it was apparent from the drop-spotting assay that the peptide CRVIIF caused the greatest regrowth response, this does not necessarily imply that it was the most effective inhibitor of HIF. The splicing efficiency of peptides from the Ssp intein may vary slightly depending on their sequence, thus resulting in different concentrations of peptide in the cell, which may therefore bias the extent of regrowth response.¹⁰ It was therefore decided that to fully validate these hits, that the binding activity was to be assessed under in vitro assay conditions.

The three lead peptides were next chemically synthesised, using Fmoc-solid phase peptide synthesis methodology, followed by in-solution cyclisation under high dilution conditions. Following this, their binding affinity was determined against the recombinantly expressed PAS-B domain of both HIF-1α and 20 using microscale thermophoresis (MST) (FIG. 1e).

The peptide CKLIIF [SEQ ID NO: 25] had the greatest affinity for both the 1α and 2α PAS-B domains, at about 2 μM, with little difference in selectivity between the two proteins. Likewise, CRLLIF [SEQ ID NO: 26] appeared to have similar binding to both isoforms, however its affinity was poorer than CKLIIF [SEQ ID NO: 25], at about 10-14 μM. In contrast, CRVIIF [SEQ ID NO: 27] had poorer affinities for both proteins, but also exhibited slight 2-fold selectivity for 1α over 2α, with 61 and 118 μM respectively. It should also be noted that the peptides CKLIIF [SEQ ID NO: 25] and CRLLIF [SEQ ID NO: 26] exhibited improved affinities for the PAS-B domain of HIF-1α, when compared with the previously reported selective inhibitor of HIF-1 dimerization, cyclo-CLLFVY [SEQ ID NO: 28].⁹

### Example 2

### Development of a Lead Compound

Following on from this, the peptides CKLIIF and CRLLIF were taken forward for further optimisation. Given the structural similarity of these peptides, it was surprising that the affinity varied between the two. It was envisaged that this difference was facilitated via either the K or R residue at position 2, or the I or L at position 4. The intermediary peptides CRLIIF [SEQ ID NO: 6] and CKLLIF [SEQ ID NO: 7], prepared by swapping one of the residues at the 2 or 4 position respectively, were therefore synthesised and tested via MST against 1α PAS-B (FIG. 2).

Interestingly, with a K_{d} of 3.8±0.4 μM, CRLIIF [SEQ ID NO: 6] was found to bind with about 3-fold greater affinity than CKLLIF [SEQ ID NO: 7]. It appeared that the exchange of R to K at position 2 had less effect than the L to I change at position 4, as the affinity for CRLIIF [SEQ ID NO: 6] resembled that of CKLIIF [SEQ ID NO: 25] (3.8 μM vs 2.5 μM), and CKLLIF [SEQ ID NO: 7] resembled CRLLIF [SEQ ID NO: 26] (12 μM vs 14 μM). The presence of a lysine residue in the peptide was found to be somewhat problematic during peptide synthesis and required orthogonal protecting methods that in this instance served to complicate solubility and the final recovery of the eventual cyclic peptide product. The poor solubility and handling of side-chain protected peptides has been described in the literature.¹¹ In order to expedite further optimisation efforts, the peptide CRLIIF [SEQ ID NO: 6] was therefore chosen as the lead peptide, as the deprotected arginine residue was found not to interfere with the cyclisation reaction, and was not a significant compromise in affinity.

### SAR Analysis by Alanine Scanning

The peptide CRLIIF [SEQ ID NO: 6, cyclic peptide #4] was next subject to alanine scanning to determine the residues critical for binding activity (FIG. 3a) [SEQ ID NO: 30-35—cyclic peptide #s 6-11]. Interestingly, it was found that all substitutions, to some extent, reduced the affinity of the peptide for the PAS-B domains. It was however clear that the C1A, I5A and F6A substitutions in particular had the greatest deleterious effect on the peptide affinity. The K_{d} for ARLIIF [SEQ ID NO: 30, cyclic peptide #9] could not be determined from this data, indicating that this residue was critical. CRLIAF [SEQ ID NO: 34, cyclic peptide #7] and CRLIIA [SEQ ID NO: 35, cyclic peptide #8] exhibited large losses in activity, with 29-fold and 24-fold reduction respectively, compared to the parent compound against HIF-1α. Of particular note is that these three residues are in a contiguous sequence, and were conserved between all three original SICLOPPS hits.

### SAR of the IFC Motif

We next aimed to optimise the peptide, and investigate the SAR with HIF-1α by making substitutions at the C1, I5 and F6 positions of the peptide in turn. First, a series of substitutions were made at the C1 position [SEQ ID Nos: 36-47, cyclic peptide #s 12a-17]. In all cases, the substitutions were considerably detrimental to peptide affinity (FIG. 3b). Incorporation of thiol-containing amino acids homocysteine and penicillamine retained much of the affinity of the parent compound, however the original cysteine residue remained the most potent derivative.

The potency imparted by the thiol motif raised the possibility of the interaction being disulfide-mediated. To counter this, all assays thus far had been performed with excess reducing agent tris(2-carboxyethyl)phosphine (TCEP) present in the buffers. Nonetheless the possibility was still considered due to the importance of the thiol moiety. As the HIF-α PAS-B domain contains multiple cysteines in internal cavities there remained the potential for disulfide bond formation within a solvent and TCEP-inaccessible region of the protein-peptide complex. Three 1α PAS-B mutants were therefore prepared with mutations C255A, C334A or C337A and the activity of the peptide CRLIIF [SEQ ID NO: 6, cyclic peptide #4] again tested by MST. It was anticipated that if a disulfide bond were to form with any of these then there would be a significant loss of binding for the peptide with the corresponding mutant. The affinity of the peptide was found to be identical to that of the wild-type protein in all cases, therefore ruling out the participation of these cysteines in binding (FIG. 3c).

Next, peptide derivatives containing unnatural amino acid substitutions at the F6 position were synthesized and their affinity investigated against the HIF-1α PAS-B domain [SEQ ID NO: 48-62, cyclic peptide #s 18-32]. In all cases the peptides retained some affinity for the protein, with mostly modest improvements or losses in binding. Notably, the introduction of more hydrophobic groups at the 4-position was preferred and found to improve the K_{d} of the peptide, with the most potent compound, the 4-iodophenylalanine derivative exhibiting a K_{d} of 850 nM (FIG. 3d). This represents a 4.5-fold improvement in affinity relative to the parent compound.

Next, peptide derivatives containing amino acid substitutions at the I5 position were synthesized and their affinity investigated against the HIF-1α PAS-B domain. Similarly to the cysteine substitutions described previously, all variants tested exhibited poorer affinities than the parent isoleucine residue [SEQ ID Nos 63-69, cyclic peptide #s 33-39] (FIG. 3e). In general, the Kd is correlated with the length of the side chain, indicating a hydrophobic driving force behind the interaction. Interestingly, the Aib derivative, whilst still exhibiting a weaker Kd than the parent compound, had markedly improved affinity over both the alanine and aminobutyric acid. This, coupled with the observation that branched-chain amino acids (Ile, Val) exhibited improved activities over their linear chain isomers (Leu and Nva, respectively) suggests a role of conformation at this position in determining affinity.

Following this, the affinity of CRLIIF(4I) [SEQ ID NO: 48 cyclic peptide #18] for HIF-2α PAS-B was also tested and the Kd found to be 1.9 μM, representing a 2.2-fold loss in Kd compared to the 1α isoform. This selectivity difference is consistent with that observed for the alanine scan derivatives determined previously (FIG. 3f).

Following in vitro characterisation and optimisation of binding, the effect of the cyclic peptides on cellular systems was assessed. A cell-based assay was developed in which the expression of yellow fluorescent protein (YFP) was put under the control of the HRE. A cassette was designed containing the YFP gene preceded by three copies of the HRE sequence. This cassette in turn was stably integrated into the chromosome of T-REx-293 cell line as described previously.¹²

The 9 most potent molecules were tested in YFP-reporter HEK cell line (FIG. 4). In these cells, hypoxia results in increased expression of YFP (directly by HIF), so a HIF inhibitor would be expected to reduce the fluorescence of these cells when incubated in hypoxia.

CRLII(4-Iodo)F [SEQ ID NO: 48 cyclic peptide #18] was taken forward as it had better water solubility than the 4-benzyl compound. The 4-iodo derivative was tested at various doses in the YFP reporter assay and showed an IC₅₀ in the above YFP assay of around 35 μM (FIG. 5)

The 4-iodo derivative was tested in a commercial cell-viability assay, and was not toxic to cells at up to twice the tested doses (FIG. 6).

The binding of 4-iodo derivative to HIF-1a was assessed by CETSA in MCF-7 cells. Stabilisation of HIF-1a by this compound can be observed at 51° C. and 54° C. (FIG. 7).

The ability of the 4-iodo derivative to disrupt the interaction of HIF-1a and HIF-1b was assessed by proximity ligation assay (PLA) in MCF-7 cells (FIG. 8).

The effect of p-iodo derivative, as well as its parent peptide (CRLIIF), on HIF target gene expression was assessed by qPCR (FIG. 9).

### Example 3

### Methods

### Construction of the HIF-2α/1α:HIF-1β RTHS

The HIF-1α:HIF-1β RTHS used in this study is the same as described previously. The HIF-2α:HIF-1β RTHS was constructed in an analogous manner to that of the HIF-1α system, utilising the HIF-2α gene encoding for residues 1-360.

### Screening

A SICLOPPS library encoding for CXXXXX [SEQ ID NO: 60] was prepared as previously described. The library mixture was transformed into electrocompetent RTHS cell lines using standard protocols. The transformation mixture was then plated onto M9 media agar plates, supplemented with 25 μg/mL kanamycin, 25 μg spectinomycin, 34 μg/mL chloramphenicol, 4 mM 3-amino-1,2,4-triazole, 50 μM IPTG and 6.5 μM arabinose. Plates were incubated for 72 h at 37° C. after which colonies were picked, grown in LB media at 37° C. overnight, and plasmids extracted using a GeneJET plasmid miniprep kit (Thermo Scientific).

### Protein Expression

Genes encoding for human HIF-1α PAS-B (238-349) and HIF-2α PAS-B (240-350) were each cloned into pET28a vectors at the BamHI and SacI restriction sites. The recombinant plasmids were next transformed into BL21(DE3)-Rosetta chemically competent cells. Cells were grown in LB media at 37° C. to an OD₆₀₀ of 0.7, after which expression was induced by addition of 0.5 mM IPTG. Following overnight expression at 16° C., cells were pelleted and resuspended in buffer containing 50 mM Tris, 150 mM NaCl, 5% glycerol, 1 mM TCEP, protease inhibitors (Roche) and 20 mM imidazole, pH 8.0. Cells were lysed by sonication, and the supernatant applied to a 1 mL Histrap FF affinity column. The His-tagged protein of interest was eluted with buffer containing 50 mM Tris, 150 mM NaCl, 5% glycerol, 1 mM TCEP and 250 mM imidazole. The protein was then loaded onto a HiLoad 16/600 superdex 75 pg size exclusion column (GE healthcare) equilibrated with assay buffer containing 50 mM Tris, 150 mM NaCl, 5% glycerol and 1 mM TCEP, pH 8.0. Fractions containing the desired protein were flash-frozen and stored at −80° C. prior to use.

### MST

HIF-α proteins were labelled with Monolith NT-647 labelling dye (Nanotemper Technologies GmbH) according to the manufacturer's instructions. MST experiments were performed on a Monolith NT.115 system (Nanotemper Technologies GmbH), in assay buffer containing 50 nM labelled protein, 10% DMSO and 0.05% TWEEN-20. MST measurements were performed using 50% LED and 50% MST power.

### Solid Phase Peptide Synthesis (General Procedure 1)

Peptides were synthesised by Fmoc solid-phase peptide synthesis using Wang resin preloaded with the first amino acid residue. Coupling and deprotection steps were performed at room temperature in a sintered funnel with agitation through the bottom of the sinter by stream of argon. Coupling solutions were prepared using Fmoc-protected amino acid (3 eq.) and HOBt hydrate (5 eq.) dissolved in DMF, to which DIC (3 eq.) was added. The mixture was stirred for 3 minutes, after which the solution was added to the resin and agitated for 1 h. The resin was washed three times with DMF, then three times DCM and finally three times Et₂O. Successful coupling was checked using the Kaiser test, and the coupling step repeated if necessary. Fmoc deprotection was carried out by agitating the resin with 20% piperidine in DMF for 20 mins. The resin was washed as before, and successful deprotection checked using the Kaiser test prior to moving on. After deprotection of the final residue, the dry resin was transferred to a 12 mL vial and stirred with TFA/TIS/H₂O (95:2.5:2.5) cocktail (10 mL per mmol peptide) for 2.5 h to cleave the peptide. The mixture was filtered through a cotton filter, and the filtrate concentrated in vacuo. Peptide was precipitated from the remaining residue with cold Et₂O. The Et₂O was removed and precipitate triturated a further two times with cold Et₂O, after which the solid was dried on a rotary evaporator. Depending on purity, the peptide was either used for subsequent reactions without further purification, or was dissolved in a H₂O:MeCN mixture (1:1) prior to purification by reverse-phase chromatography.

The following protected amino acids were used in couplings unless otherwise specifically stated: Fmoc-Ala-OH·2H₂O, Fmoc-Cys(StBu)-OH, Fmoc-Phe-OH, Fmoc-Ile-OH, Fmoc-Lys(Boc), Fmoc-Leu-OH, Fmoc-Met-OH, Fmoc-Arg(Pbf)-OH, Fmoc-Ser(tBu)-OH, Fmoc-Thr(tBu)-OH, Fmoc-Val-OH, Fmoc-Tyr(tBu)-OH.

### Peptide Cyclisation (General Procedure 2)

Linear peptide, HATU (1.5 eq) and HOAt (3 eq) were dissolved in DMF (1 mL DMF per mg peptide). To the stirring mixture was added DIPEA (5 eq) and the reaction was left to stir at room temperature for 4-18 h. The reaction was monitored by LCMS. After reaction completion, the mixture was concentrated on a rotary evaporator and the remaining residue purified by reverse-phase chromatography if required, otherwise the residue was telescoped to the next reaction.

### StBu Deprotection (General Procedure 3)

The peptide and DTT (10 eq) were dissolved in 1 mL DMF. To the mixture was added 1M (NH₄)₂CO₃ solution (10 eq) in H₂O. The reaction was stirred at room temperature for 30 mins. The reaction mixture was filtered through a 0.2 μm Teflon syringe filter (Thermo Scientific) and purified directly by reverse-phase preparative HPLC.

### Reverse Phase Chromatography

Reverse phase chromatography was performed on a Biotage Isolera One system, using linear gradients of solvents A (0.1% TFA/H₂O) and B (0.1% TFA/MeCN). Peptide solutions were loaded into Biotage SNAP Ultra C18 30 g columns dissolved in either a H₂O:MeCN mixture where possible, or DMF. Methods were run at 50 mL/min. The following general methods were used:

### Method A:

| Column | % A | % B |
|---|---|---|
| 1 | 95 | 5 |
| 10 | 40 | 60 |
| 2 | 40 | 60 |

### Method B:

| Column | % A | % B |
|---|---|---|
| 1 | 70 | 30 |
| 10 | 10 | 90 |
| 2 | 10 | 90 |

### Preparative HPLC

Preparative HPLC was performed on a Waters 1525 HPLC system using linear gradients of solvents A (0.1% TFA/H₂O) and B (0.1% TFA/MeCN). Peptides were purified by preparative HPLC with a Waters Atlantis T3 column (5.0 μm particle size, 19×100 mm) at 17 mL/min flow rate.

The following general methods were used:

### Method A:

| Time (min) | % A | % B |
|---|---|---|
| 0.00 | 95 | 5 |
| 1.00 | 95 | 5 |
| 15.00 | 10 | 90 |
| 20.00 | 10 | 90 |

### Method B:

| Time (min) | % A | % B |
|---|---|---|
| 0.00 | 80 | 20 |
| 1.00 | 80 | 20 |
| 15.00 | 10 | 90 |
| 20.00 | 10 | 90 |

### Analytical HPLC

Analytical HPLC was performed on an Agilent 1260 Infinity II HPLC system using linear gradients of solvents A (0.1% TFA/H₂O) and B (0.1% TFA/MeCN). Peptides were eluted through an Agilent Poroshell 120 EC-C18 column (2.7 μm particle size, 3.0×100 mm) at 0.625 ml/min flow rate. Samples were injected as a 5% MeCN/H₂O solution. Samples were detected using UV absorbance at 220 nm and 280 nm.

The following general method was used:

### Method A:

| Time (min) | % A | % B |
|---|---|---|
| 0.00 | 95 | 5 |
| 20.00 | 0 | 100 |
| 22.00 | 0 | 100 |

### Example 4

### Production of Cyclic Peptides

### cyclo-cys lys leu ile ile phe (SEQ ID NO: 25, Cyclic Peptide #1) [Included for Comparative Purposes Only]

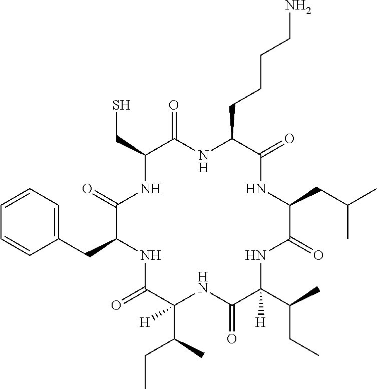 [SMILES: CC[C@H](C)[C@@H]1NC(=O)[C@H](CC(C)C)NC(=O)[C@H](CCCCN)NC(=O)[C@H](CS)NC(=O)[C@H](Cc2ccccc2)NC(=O)[C@H]([C@@H](C)CC)NC1=O]

The linear peptide ile ile phe cys(StBu) lys(Cbz) leu was synthesised from Fmoc-Leu-Wang resin (Novabiochem) on a 0.2 mmol scale following general procedure 0, using Fmoc-Lys(Cbz)-OH instead of Fmoc-Lys(Boc)-OH. After precipitation from ether, the peptide was purified by RPC (method B) and lyophilised. The white solid was then dissolved in DMF and cyclised following general procedure 0. The peptide was concentrated, deprotected following general procedure 0, purified by RPC (method A) and lyophilised. The subsequent white solid was left stirring in 2 mL TFA over 3 days at room temperature, after which the product was purified by RP-HPLC (method A) and lyophilised to obtain 1 as a white solid (10 mg, 7% overall yield). LCMS Rₜ: 1.71 min, Analytical HPLC Rₜ: 11.283 min (91% purity), LRMS m/z (ESI⁺): 718.6 [M+H] (100%), 360.0 [M+2H] (85%), HRMS m/z (ESI⁺): calc. for C₃₆H₆₀N₇O₆S ([M+H⁺]) 718.4320, found 718.4326.

### cyclo-cys arg leu leu ile phe (SEQ ID NO: 26, cyclic Peptide #2) [Included for Comparative Purposes Only]

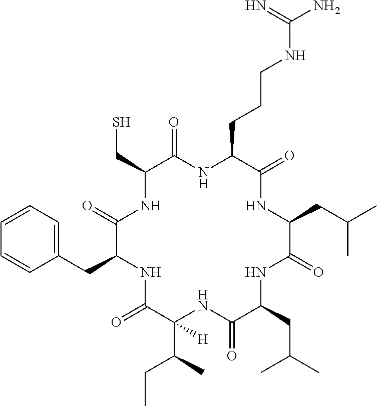 [SMILES: CC[C@H](C)[C@@H]1NC(=O)[C@H](CC(C)C)NC(=O)[C@H](CC(C)C)NC(=O)[C@H](CCCNC(=N)N)NC(=O)[C@H](CS)NC(=O)[C@H](Cc2ccccc2)NC1=O]

The linear peptide leu ile phe cys(StBu) arg leu was synthesised from Fmoc-Leu-Wang resin (Novabiochem) on a 0.2 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 2 as a white solid (83 mg, 56% overall yield). LCMS Rₜ: 1.79 min, Analytical HPLC Rₜ: 12.298 min (95% purity), LRMS m/z (ESI⁺): 834.7 [M+H] (100%), 418.1 [M+2H] (85%), HRMS m/z (ESI⁺): calc. for C₃₆H₆₀N₉O₆S ([M+H⁺]) 746.4382, found 746.4366.

### cyclo-cys arg val ile ile phe (SEQ ID NO: 27, cyclic peptide #3) [Included for Comparative Purposes Only]

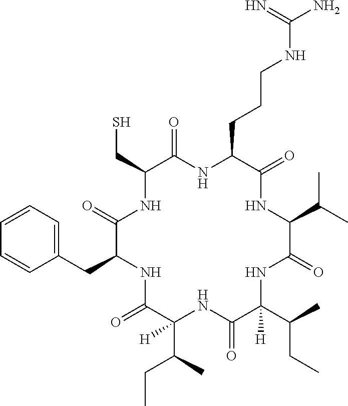 [SMILES: CC[C@H](C)[C@@H]1NC(=O)[C@H](C(C)C)NC(=O)[C@H](CCCNC(=N)N)NC(=O)[C@H](CS)NC(=O)[C@H](Cc2ccccc2)NC(=O)[C@H]([C@@H](C)CC)NC1=O]

The linear peptide ile ile phe cys(StBu) arg val was synthesised from Fmoc-Val-Wang resin (Novabiochem) on a 0.2 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 3 as a white solid (18 mg, 12% overall yield). LCMS Rₜ: 1.75 min, Analytical HPLC Rₜ: 11.458 min (96% purity), LRMS m/z (ESI⁺): 732.7 [M+H] (100%), 366.9 [M+2H] (85%), HRMS m/z (ESI⁺): calc. for C₃₅H₅₈N₉O₆S ([M+H⁺]) 732.4225, found 732.4217.

### cyclo-cys agr leu ile ile phe (SEQ ID NO: 6, Cyclic Peptide #4)

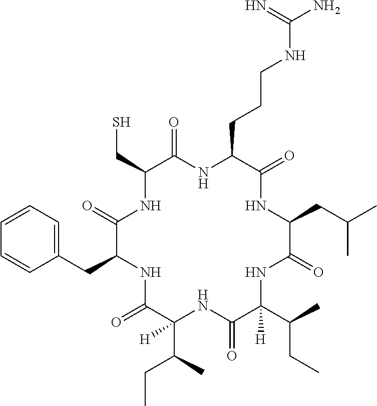 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccccc2)NC(=O)[C@H]([C@@H](C)CC)NC1=O]

The linear peptide ile ile phe cys(StBu) arg leu was synthesised from Fmoc-Leu-Wang resin (Novabiochem) on a 0.2 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 4 as a white solid (60 mg, 40% overall yield). LCMS Rₜ: 1.88 min, Analytical HPLC Rₜ: 12.499 min (99% purity), LRMS m/z (ESI⁺): 746.6 [M+H] (100%), 374.0 [M+2H] (90%), HRMS m/z (ESI⁺): calc. for C₃₆H₆₀N₉O₆S ([M+H⁺]) 746.4382, found 746.4380.

### cyclo-cys lys leu ile ile phe (SEQ ID NO: 7, Cyclic Peptide #5)

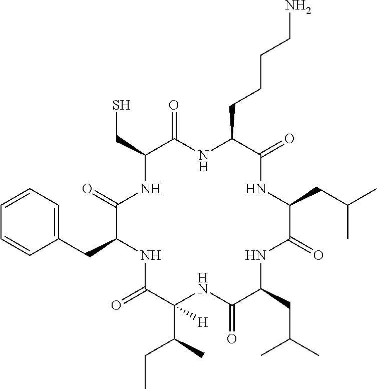 [SMILES: CC[C@H](C)[C@@H]1NC(=O)[C@H](CC(C)C)NC(=O)[C@H](CC(C)C)NC(=O)[C@H](CCCCN)NC(=O)[C@H](CS)NC(=O)[C@H](Cc2ccccc2)NC1=O]

The linear peptide ile ile phe cys(StBu) lys(Cbz) leu was synthesised from Fmoc-Leu-Wang resin (Novabiochem) on a 0.2 mmol scale following general procedure 0, using Fmoc-Lys(Cbz)-OH instead of Fmoc-Lys(Boc)-OH. After precipitation from ether, the peptide was purified by RPC (method B) and lyophilised. The white solid was then dissolved in DMF and cyclised following general procedure 0. The peptide was concentrated, deprotected following general procedure 0, purified by RPC (method A) and lyophilised. The subsequent white solid was left stirring in 2 ml TFA over 3 days at room temperature, after which the product was was purified by RP-HPLC (method A) and lyophilised to obtain 1 as a white solid (23 mg, 16% overall yield). LCMS Rₜ: 1.89 min, Analytical HPLC Rₜ: 12.115 min (97% purity), LRMS m/z (ESI⁺): 719.1 [M+H] (100%), 360.1 [M+2H] (39%), HRMS m/z (ESI⁺): calc. for C₃₆H₆₀N₇O₆S ([M+H−⁺]) 718.4320, found 718.4325.

### cyclo-cys agr leu ala ile phe (SEQ ID NO: 33, Cyclic Peptide #6) [Included for Comparative Purposes Only]

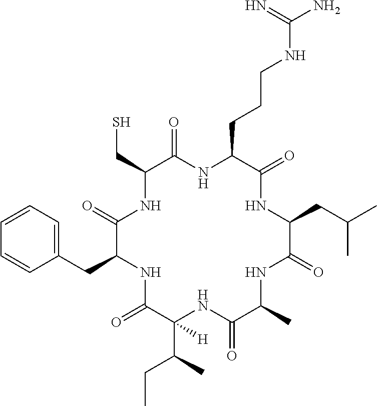 [SMILES: CC[C@H](C)[C@@H]1NC(=O)[C@H](C)NC(=O)[C@H](CC(C)C)NC(=O)[C@H](CCCNC(=N)N)NC(=O)[C@H](CS)NC(=O)[C@H](Cc2ccccc2)NC1=O]

The linear peptide ala ile phe cys(StBu) arg leu was synthesised from Fmoc-Leu-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 6 as a white solid (23 mg, 33% overall yield). LCMS Rₜ: 1.69 min, Analytical HPLC Rₜ: 10.659 min (99% purity), LRMS m/z (ESI⁺): 704.7 [M+H] (100%), 353.0 [M+2H] (75%), HRMS m/z (ESI⁺): calc. for C₃₃H₅₄N₉O₆S ([M+H⁺]) 704.3912, found 704.3905.

### cyclo-cys agr leu ile ala phe (SEQ ID NO: 34, Cyclic Peptide #7) [Included for Comparative Purposes Only]

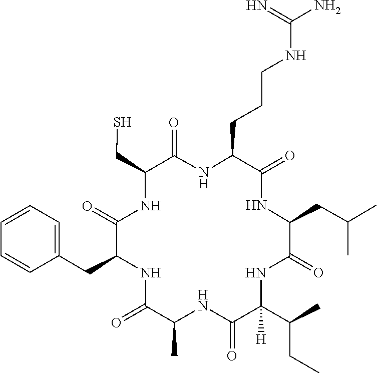 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccccc2)NC(=O)[C@H](C)NC1=O]

The linear peptide ile ala phe cys(StBu) arg leu was synthesised from Fmoc-Leu-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 7 as a white solid (10 mg, 14% overall yield). LCMS Rₜ: 1.74 min, Analytical HPLC Rₜ: 10.937 min (93% purity), LRMS m/z (ESI⁺): 353.1 [M+2H] (100%), 704.7 [M+H] (60%), HRMS m/z (ESI⁺): calc. for C₃₃H₅₄N₉O₆S ([M+H⁺]) 704.3912, found 704.3907.

### cyclo-cys agr leu ile ile ala (SEQ ID NO: 35, Cyclic Peptide #8) [Included for Comparative Purposes Only]

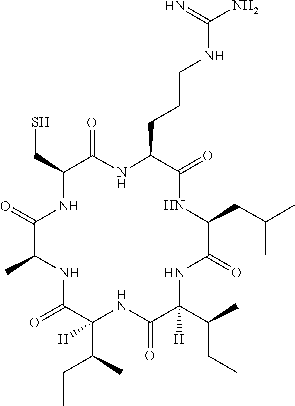 [SMILES: CC[C@H](C)[C@@H]1NC(=O)[C@H](CC(C)C)NC(=O)[C@H](CCCNC(=N)N)NC(=O)[C@H](CS)NC(=O)[C@H](C)NC(=O)[C@H]([C@@H](C)CC)NC1=O]

The linear peptide ile ile ala cys(StBu) arg leu was synthesised from Fmoc-Leu-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 8 as a white solid (31 mg, 46% overall yield). LCMS Rₜ: 1.71 min, Analytical HPLC Rₜ: 10.493 min (86% purity), LRMS m/z (ESI⁺): 336.1 [M+2H] (100%), 237.1 [M+2H+Na] (70%), 670.7 [M+H] (50%), HRMS m/z (ESI⁺): calc. for C₃₀H₅₆N₉O₆S ([M+H⁺]) 670.4069, found 670.4065.

### cyclo-ala agr leu ile ile phe (SEQ ID NO: 30, Cyclic Peptide #9) [Included for Comparative Purposes Only]

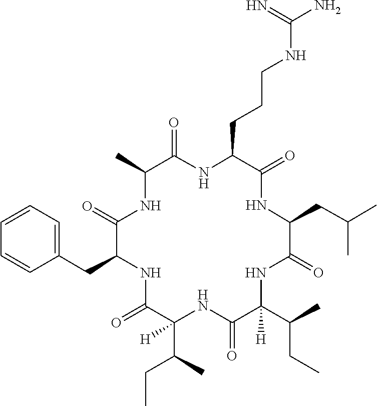 [SMILES: CC[C@H](C)[C@@H]1NC(=O)[C@H](CC(C)C)NC(=O)[C@H](CCCNC(=N)N)NC(=O)[C@H](C)NC(=O)[C@H](Cc2ccccc2)NC(=O)[C@H]([C@@H](C)CC)NC1=O]

The linear peptide ile ile phe ala arg leu was synthesised from Fmoc-Leu-Wang resin (Novabiochem) on a 0.1 mmol scale following general procedure 0. After precipitation from ether, the peptide was dissolved in DMF and cyclised following general procedure 0. The product was purified by RP-HPLC (method A) and lyophilised to obtain 9 as a white solid (33 mg, 46% overall yield). LCMS Rₜ: 1.91 min, Analytical HPLC Rₜ: 12.120 min (94% purity) LRMS m/z (ESI⁺): 358.2 [M+2H] (100%), 714.8 [M+H] (80%), HRMS m/z (ESI⁺): calc. for C₃₆H₆₀N₉O₆ ([M+H⁺]) 714.4661, found 714.4657.

### cyclo-cys ala leu ile ile phe (SEQ ID NO: 31, Cyclic Peptide #10) [Included for Comparative Purposes Only]

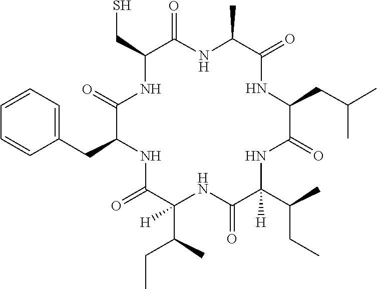 [SMILES: CC[C@H](C)[C@@H]1NC(=O)[C@H](CC(C)C)NC(=O)[C@H](C)NC(=O)[C@H](CS)NC(=O)[C@H](Cc2ccccc2)NC(=O)[C@H]([C@@H](C)CC)NC1=O]

The linear peptide ile ile ala cys(StBu) arg leu was synthesised from Fmoc-Leu-Wang resin (Novabiochem) on a 0.2 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 10 as a white solid (13 mg, 10% overall yield). LCMS Rₜ: 2.25 min, Analytical HPLC Rₜ: 14.566 min (90% purity), LRMS m/z (ESI⁺): 350.4 [M+H+K] (100%), 683.6 [M+Na] (85%), 661.5 [M+H] (30%), HRMS m/z (ESI⁺): calc. for C₃₃H₅₂N₆O₆SNa ([M+Na⁺]) 683.3561, found 683.3551.

### cyclo-cys agr ala ile ile phe (SEQ ID NO: 32, Cyclic Peptide #11) [Included for Comparative Purposes Only]

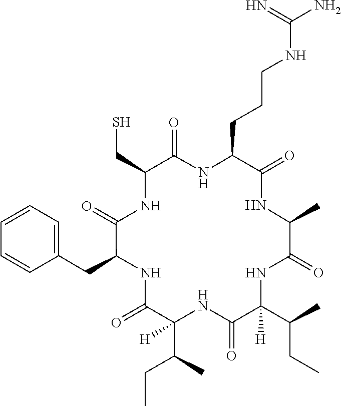 [SMILES: CC[C@H](C)[C@@H]1NC(=O)[C@H](C)NC(=O)[C@H](CCCNC(=N)N)NC(=O)[C@H](CS)NC(=O)[C@H](Cc2ccccc2)NC(=O)[C@H]([C@@H](C)CC)NC1=O]

The linear peptide ile ile ala cys(StBu) arg leu was synthesised from Fmoc-Leu-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 11 as a white solid (7 mg, 10% overall yield). LCMS Rₜ: 1.76 min, Analytical HPLC Rₜ: 11.081 min (90% purity), LRMS m/z (ESI⁺): 353.1 [M+2H] (100%), 704.7 [M+H] (60%), HRMS m/z (ESI⁺): calc. for C₃₃H₅₄N₉O₆S ([M+H⁺]) 704.3912, found 704.3905.

### H₂N-phe (Pen) arg leu ile ile-OH (SEQ ID NO: 36, Cyclic Peptide #12a) [Included for Comparative Purposes Only]

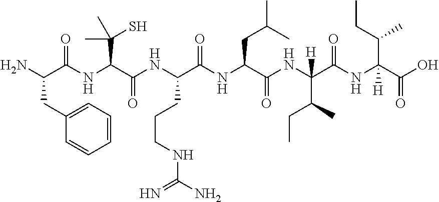 [SMILES: CC[C@H](C)[C@H](NC(=O)[C@@H](NC(=O)[C@H](CC(C)C)NC(=O)[C@H](CCCNC(=N)N)NC(=O)[C@@H](NC(=O)[C@@H](N)Cc1ccccc1)C(C)(C)S)[C@@H](C)CC)C(=O)O]

The peptide phe Pen arg leu ile ile was synthesised from Fmoc-Ile-Wang resin (Novabiochem) on a 0.1 mmol scale following general procedure 0, using Fmoc-Pen(Trt)-OH for the penicillamine coupling. Following precipitation from ether, the peptide as the TFA salt was dried and isolated as an off-white solid (72 mg, 79%) and used without further purification. LCMS Rₜ: 1.84 min, LRMS m/z (ESI⁺): 397.2 [M+2H] (100%), 792.9 [M+H] (10%).

### H₂N-ile ile phe (Pen(SPy)) arg leu-OH (SEQ ID NO: 37, Cyclic Peptide #12b) [Included for Comparative Purposes Only]

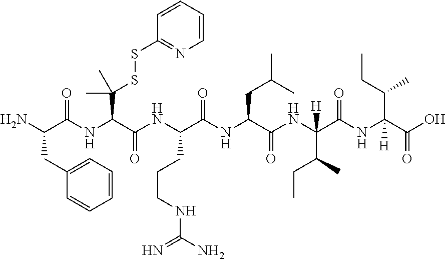 [SMILES: CC[C@H](C)[C@H](NC(=O)[C@@H](NC(=O)[C@H](CC(C)C)NC(=O)[C@H](CCCNC(=N)N)NC(=O)[C@@H](NC(=O)[C@@H](N)Cc1ccccc1)C(C)(C)SSc1ccccn1)[C@@H](C)CC)C(=O)O]

The peptide 12a (72 mg, 0.079 mmol) was dissolved in MeOH (1 mL). To the stirring mixture was added dropwise a solution of aldrithiol-2 (35 mg, 0.159 mmol, 2 eq) in MeOH (1 mL). The reaction was left to stir at room temperature for 30 min after which no starting material was detected by LCMS. The reaction mixture was concentrated to dryness on a rotary evaporator, and to the residue was added Et₂O (2 mL). The yellow ether solution was removed and the precipitate washed a further two times with Et₂O. The solid 12b was dried and isolated as an off-white solid (70 mg, 98%) and used without further purification. LCMS Rₜ: 1.94 min, LRMS m/z (ESI⁺): 451.8 [M+2H] (100%).

### cyclo-Pen(SPy) agr leu ile ile phe (SEQ ID NO: 38, Cyclic Peptide #12c) [Included for Comparative Purposes Only]

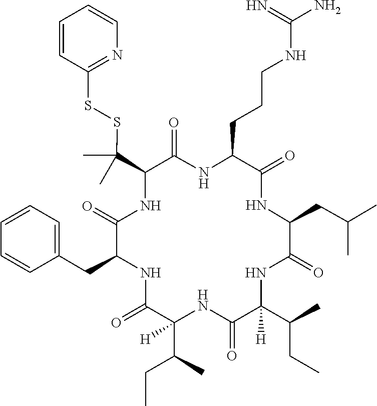 [SMILES: CC[C@H](C)[C@@H]1NC(=O)[C@H](CC(C)C)NC(=O)[C@H](CCCNC(=N)N)NC(=O)[C@H](C(C)(C)SSc2ccccn2)NC(=O)[C@H](Cc2ccccc2)NC(=O)[C@H]([C@@H](C)CC)NC1=O]

The peptide was synthesised from 12b (70 mg, 0.078 mmol), HATU (41 mg), HOAt (25 mg) and DIPEA (47 μL) in DMF (90 mL) following general procedure 0. The product was purified by RP-chromatography (method B) and lyophilised to obtain 12c as a white solid (22 mg, 32%). LCMS Rₜ: 2.07, LRMS m/z (ESI⁺) 442.6 [M+2H] (100%), 884.0 [M+H] (10%).

### cyclo-Pen agr leu ile ile phe (SEQ ID NO: 39, Cyclic Peptide #12d) [Included for Comparative Purposes Only]

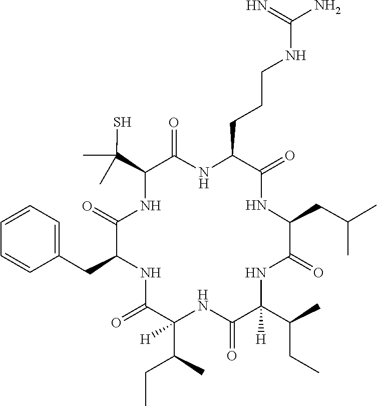 [SMILES: CC[C@H](C)[C@@H]1NC(=O)[C@H](CC(C)C)NC(=O)[C@H](CCCNC(=N)N)NC(=O)[C@H](C(C)(C)S)NC(=O)[C@H](Cc2ccccc2)NC(=O)[C@H]([C@@H](C)CC)NC1=O]

The peptide was synthesised from 12c (22 mg, 0.025 mmol), DTT (77 mg) and 1M aq. (NH₄)₂CO₃ (0.5 mL) in DMF (0.5 mL) following general procedure 0. The product was purified by RP-HPLC (method A) and lyophilised to obtain 12d as a white solid (15 mg, 78%). LCMS Rₜ: 1.95 min, Analytical HPLC Rₜ: 13.384 (81% purity) LRMS m/z (ESI⁺): 388.1 [M+2H] (100%), 774.9 [M+H] (55%), HRMS m/z (ESI⁺): calc. for C₃₈H₆₄N₉O₆S ([M+H⁺]) 774.4695, found 774.4704.

### H₂N-phe (hcys) arg leu ile ile-OH (SEQ ID NO:40, Cyclic Peptide #13a) [Included for Comparative Purposes Only]

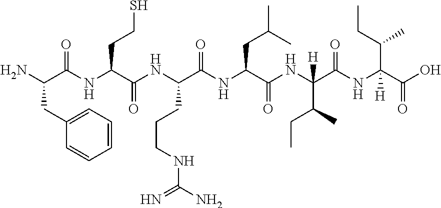 [SMILES: CC[C@H](C)[C@H](NC(=O)[C@@H](NC(=O)[C@H](CC(C)C)NC(=O)[C@H](CCCNC(=N)N)NC(=O)[C@H](CCS)NC(=O)[C@@H](N)Cc1ccccc1)[C@@H](C)CC)C(=O)O]

The peptide phe (hcys) arg leu ile ile was synthesised from Fmoc-Ile-Wang resin (Novabiochem) on a 0.1 mmol scale following general procedure 0, using Fmoc-hCys(Trt)-OH for the homocysteine coupling. The product was purified by RP-chromatography (method A) and lyophilised to obtain 13a as an off-white solid (47 mg, 60%). LCMS Rₜ: 1.81 min, LRMS m/z (ESI⁺): 390.0 [M+2H] (100%), 778.7 [M+H] (20%).

### H₂N-ile ile phe (hC(SPy)) arg leu-OH (SEQ ID NO: 41, Cyclic Peptide #13b) [Included for Comparative Purposes Only]

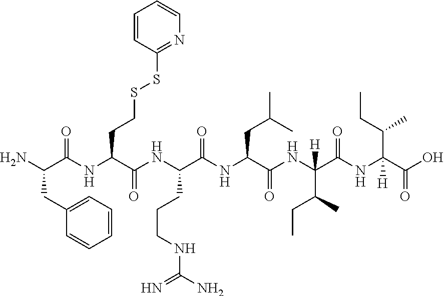 [SMILES: CC[C@H](C)[C@H](NC(=O)[C@@H](NC(=O)[C@H](CC(C)C)NC(=O)[C@H](CCCNC(=N)N)NC(=O)[C@H](CCSSc1ccccn1)NC(=O)[C@@H](N)Cc1ccccc1)[C@@H](C)CC)C(=O)O]

The peptide 13a (47 mg, 0.06 mmol) was dissolved in MeOH (1 mL). To the stirring mixture was added dropwise a solution of aldrithiol-2 (26 mg, 0.12 mmol, 2 eq) in MeOH (1 mL). The reaction was left to stir at room temperature for 30 min after which no starting material was detected by LCMS. The reaction mixture was concentrated to dryness on a rotary evaporator, and to the residue was added Et₂O (2 mL). The yellow ether solution was removed and the precipitate washed a further two times with Et₂O. The solid 13b was dried and isolated as an off-white solid (36 mg, 68%) and used without further purification. LCMS Rₜ: 1.96 min, LRMS m/z (ESI⁺): 444.7 [M+2H] (100%).

### cyclo-hcys(SPy) arg leu ile ile phe (SEQ ID NO: 42, Cyclic Peptide #13c) [Included for Comparative Purposes Only]

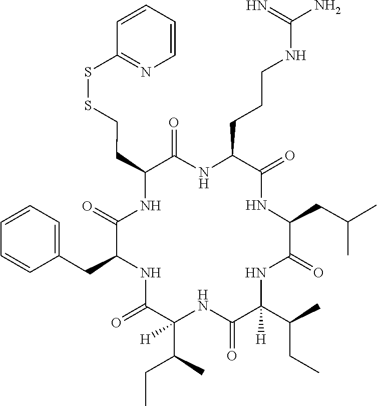 [SMILES: CC[C@H](C)[C@@H]1NC(=O)[C@H](CC(C)C)NC(=O)[C@H](CCCNC(=N)N)NC(=O)[C@H](CCSSc2ccccn2)NC(=O)[C@H](Cc2ccccc2)NC(=O)[C@H]([C@@H](C)CC)NC1=O]

The peptide was synthesised from 13b (36 mg, 0.041 mmol), HATU (19 mg), HOAt (11 mg) and DIPEA (21 μL) in DMF (40 mL) following general procedure 0. The product was purified by RP-HPLC (method B) and lyophilised to obtain 13c as a white solid (12 mg, 34%). LCMS Rₜ: 2.06 min, LRMS m/z (ESI⁺) 435.6 [M+2H] (100%), 869.7 [M+H] (20%).

### cyclo-hcys agr leu ile ile phe (SEQ ID NO: 43, Cyclic Peptide #13d) [Included for Comparative Purposes Only]

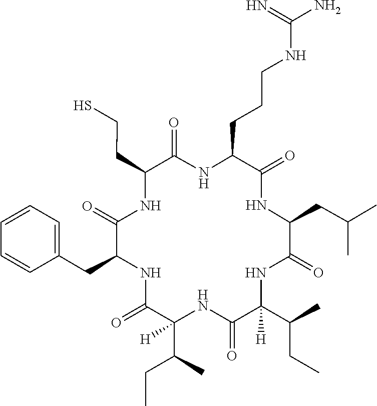 [SMILES: CC[C@H](C)[C@@H]1NC(=O)[C@H](CC(C)C)NC(=O)[C@H](CCCNC(=N)N)NC(=O)[C@H](CCS)NC(=O)[C@H](Cc2ccccc2)NC(=O)[C@H]([C@@H](C)CC)NC1=O]

The peptide was synthesised from 13c (12 mg, 0.025 mmol), DTT (77 mg) and 1M aq. (NH₄)₂CO₃ (0.5 mL) in DMF (0.5 mL) following general procedure 0. The product was purified by RP-HPLC (method A) and lyophilised to obtain 13d as a white solid (6 mg, 8%). LCMS Rₜ: 1.95 min, Analytical HPLC Rₜ: 12.750 min (84% purity) LRMS m/z (ESI⁺): 381.1 [M+2H] (100%), 760.7 [M+H] (90%), HRMS m/z (ESI⁺): calc. for C₃₇H₆₂N₉O₆S ([M+H⁺]) 760.4538, found 760.4533.

### cyclo-met agr leu ile ile phe (SEQ ID NO: 44, Cyclic Peptide #14) [Included for Comparative Purposes Only]

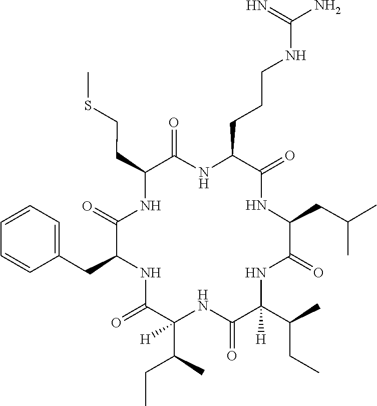 [SMILES: CC[C@H](C)[C@@H]1NC(=O)[C@H](CC(C)C)NC(=O)[C@H](CCCNC(=N)N)NC(=O)[C@H](CCSC)NC(=O)[C@H](Cc2ccccc2)NC(=O)[C@H]([C@@H](C)CC)NC1=O]

The linear peptide phe met arg leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The product was purified by RP-HPLC (method A) and lyophilised to obtain 14 as a white solid (47 mg, 61% overall yield). LCMS Rₜ: 2.31 min, Analytical HPLC Rₜ: 12.926 min (79% purity), LRMS m/z (ESI⁺): 388.1 [M+2H] (100%), 774.9 [M+2H] (40%), HRMS m/z (ESI⁺): calc. for C₃₈H₆₄N₉O₆S ([M+H⁺]) 774.4695, found 774.4687.

### cyclo-ser agr leu ile ile phe (SEQ ID NO: 45, Cyclic Peptide #15) [Included for Comparative Purposes Only]

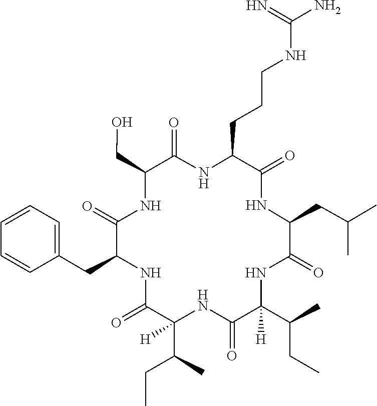 [SMILES: CC[C@H](C)[C@@H]1NC(=O)[C@H](CC(C)C)NC(=O)[C@H](CCCNC(=N)N)NC(=O)[C@H](CO)NC(=O)[C@H](Cc2ccccc2)NC(=O)[C@H]([C@@H](C)CC)NC1=O]

The linear peptide phe ser arg leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The product was purified by RP-HPLC (method A) and lyophilised to obtain 15 as a white solid (16 mg, 22% overall yield). LCMS Rₜ: 2.13 min, Analytical HPLC Rₜ: 11.500 min (95% purity), LRMS m/z (ESI⁺): 366.1 [M+2H] (100%), 730.8 [M+H] (40%), HRMS m/z (ESI⁺): calc. for C₃₆H₆₀N₉O₇ ([M+H⁺]) 730.4610, found 730.4616.

### cyclo-thr agr leu ile ile phe (SEQ ID NO: 46, Cyclic Peptide #16) [Included for Comparative Purposes Only]

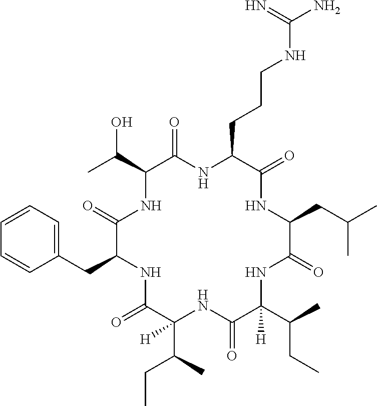 [SMILES: CC[C@H](C)[C@@H]1NC(=O)[C@H](CC(C)C)NC(=O)[C@H](CCCNC(=N)N)NC(=O)[C@H](C(C)O)NC(=O)[C@H](Cc2ccccc2)NC(=O)[C@H]([C@@H](C)CC)NC1=O]

The linear peptide phe thr agr leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The product was purified by RP-HPLC (method A) and lyophilised to obtain 16 as a white solid (26 mg, 35% overall yield). LCMS Rₜ: 2.17 min, Analytical HPLC Rₜ: 11.838 min (94% purity), LRMS m/z (ESI⁺): 373.1 [M+2H] (100%), 744.8 [M+H] (50%), HRMS m/z (ESI⁺): calc. for C₃₇H₆₂N₉O₇ ([M+H⁺]) 744.4767, found 744.4763.

### cyclo-(methyl)cys agr leu ile ile phe (SEQ ID NO: 47, Cyclic Peptide #17) [Included for Comparative Purposes Only]

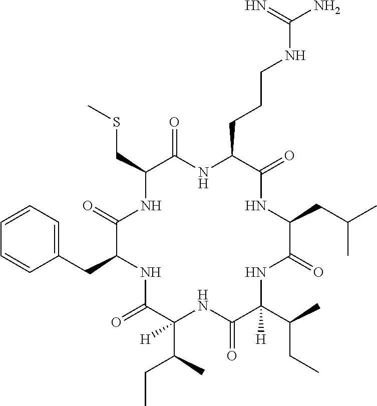 [SMILES: CC[C@H](C)[C@@H]1NC(=O)[C@H](CC(C)C)NC(=O)[C@H](CCCNC(=N)N)NC(=O)[C@H](CSC)NC(=O)[C@H](Cc2ccccc2)NC(=O)[C@H]([C@@H](C)CC)NC1=O]

To a stirring solution of cys agr leu ile ile phe (4) (8.0 mg, 0.011 mmol, 1 eq.) and MeI (1 μL, 2.3 mg, 0.016 mmol, 1.5 eq.) in 1 mL MeCN, was added DIPEA (2.8 μL, 2.1 mg, 0.016 mmol, 1.5 eq.). The mixture was stirred at room temperature for 3 hours, after which the product was purified by RP-HPLC (method A) and lyophilised to obtain 17 as a white solid (7 mg, 76% yield). LCMS Rₜ: 1.91 min, Analytical HPLC Rₜ: 12.722 min (89% purity), LRMS m/z (ESI⁺): 760.6 [M+H] (100%), 381.0 [M+2H] (75%), HRMS m/z (ESI⁺): calc. for C₃₇H₆₂N₉O₆S ([M+H⁺]) 760.4538, found 760.4542.

### cyclo-cys agr leu ile ile (4-I)phe (SEQ ID NO: 48, Cyclic Peptide #18)

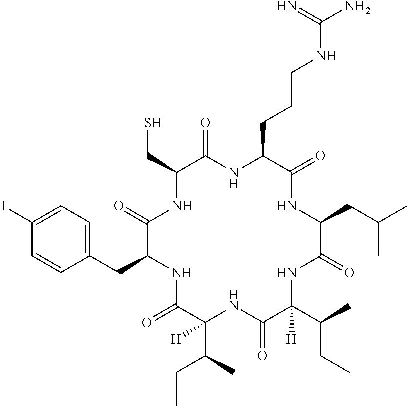 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccc(I)cc2)NC(=O)[C@H]([C@@H](C)CC)NC1=O]

The linear peptide (4-I)phe cys(StBu) agr leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 18 as a white solid (46 mg, 53% overall yield). LCMS Rₜ: 2.49 min, Analytical HPLC Rₜ: 13.865 min (99% purity), LRMS m/z (ESI⁺): 437.1 [M+2H] (100%), 872.7 [M+H] (60%), HRMS m/z (ESI⁺): calc. for C₃₆H₅₉IN₉O₆S ([M+H⁺]) 872.3354, found 872.3365.

### cyclo-cys agr leu ile ile (4-Br)phe (SEQ ID NO: 49, Cyclic Peptide #19)

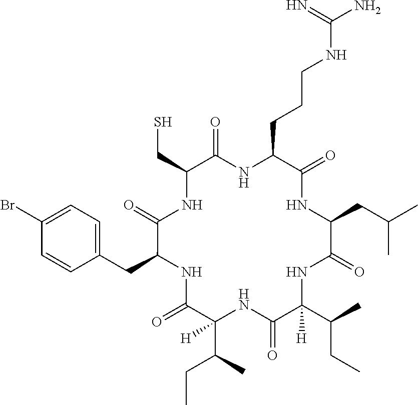 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccc(Br)cc2)NC(=O)[C@H]([C@@H](C)CC)NC1=O]

The linear peptide (4-Br)phe cys(StBu) agr leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 18 as a white solid (48 mg, 59% overall yield). LCMS Rₜ: 2.01 min, Analytical HPLC Rₜ: 13.592 min (94% purity), LRMS m/z (ESI⁺): 826.7 [M+H, ⁸¹Br] (100%), 824.8 [M+H, ⁷⁹Br] (95%), HRMS m/z (ESI⁺): calc. for C₃₆H₅₉BrN₉O₆S ([M+H⁺]) 824.3492/826.3472, found 824.3487/826.3477.

### cyclo-cys agr leu ile ile (4-CF₃)phe (SEQ ID NO: 50, Cyclic Peptide #20)

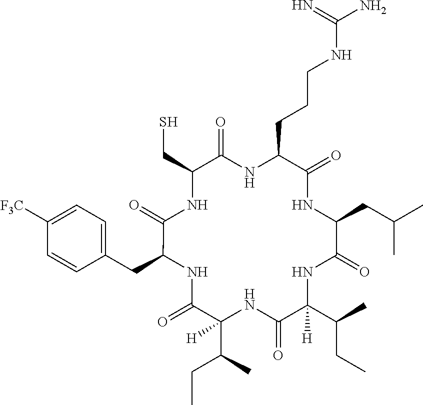 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccc(C(F)(F)F)cc2)NC(=O)[C@H]([C@@H](C)CC)NC1=O]

The linear peptide (4-CF₃)phe cys(StBu) agr leu ile ile was synthesised from Fmoc-Ile-Wang resin (Novabiochem) on a 0.05 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 20 as a white solid (12 mg, 30% overall yield). LCMS Rₜ: 2.01 min, Analytical HPLC Rₜ: 13.742 min (99% purity), LRMS m/z (ESI⁺): 408.1 [M+2H] (100%), 814.8 [M+H] (80%), HRMS m/z (ESI⁺): calc. for C₃₇H₅₉F₃N₉O₆S ([M+H⁺]) 814.4256, found 814.4256.

### cyclo-cys agr leu ile ile (4-Cl)phe (SEQ ID NO: 51, Cyclic Peptide #21)

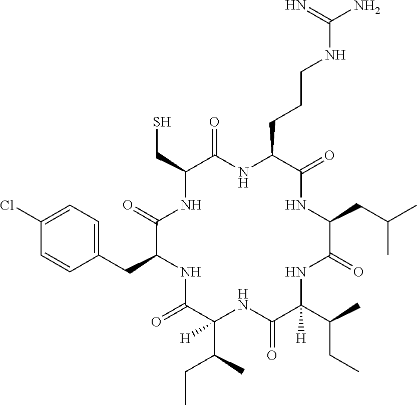 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccc(Cl)cc2)NC(=O)[C@H]([C@@H](C)CC)NC1=O]

The linear peptide (4-Cl)phe cys(StBu) agr leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 21 as a white solid (52 mg, 67% overall yield). LCMS Rₜ: 1.99 min, Analytical HPLC Rₜ: 13.327 min (82% purity), LRMS m/z (ESI⁺): 391.2 [M+2H] (100%), 780.8 [M+H] (75%), HRMS m/z (ESI⁺): calc. for C₃₆H₅₉ClN₉O₆S ([M+H⁺]) 780.3998, found 780.3978.

### cyclo-cys agr leu ile ile hphe (SEQ ID NO: 52, Cyclic Peptide #22)

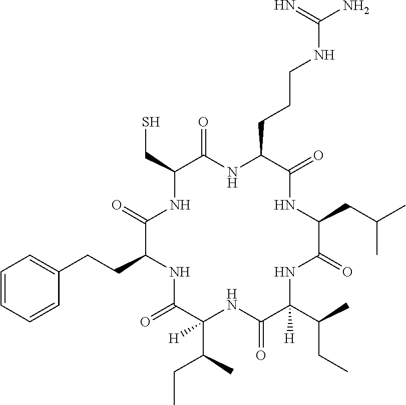 [SMILES: CC[C@H](C)[C@@H]1NC(=O)[C@H](CC(C)C)NC(=O)[C@H](CCCNC(=N)N)NC(=O)[C@H](CS)NC(=O)[C@H](CCc2ccccc2)NC(=O)[C@H]([C@@H](C)CC)NC1=O]

The linear peptide hphe cys(StBu) agr leu ile ile was synthesised from Fmoc-Ile-Wang resin (Novabiochem) on a 0.05 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 22 as a white solid (14 mg, 36% overall yield). LCMS Rₜ: 1.93 min, Analytical HPLC Rₜ: 13.089 min (85% purity), LRMS m/z (ESI⁺): 381.1 [M+2H] (100%), 760.8 [M+H] (45%), HRMS m/z (ESI⁺): calc. for C₃₇H₆₂N₉O₆S ([M+H⁺]) 760.4538, found 760.4538.

### cyclo-cys agr leu ile ile (ala(1-naph)) (SEQ ID NO: 53, Cyclic Peptide #23)

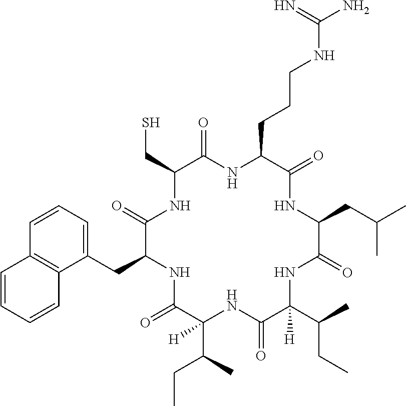 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2cccc3ccccc23)NC(=O)[C@H]([C@@H](C)CC)NC1=O]

The linear peptide (ala(1-naph)) cys(StBu) agr leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 23 as a white solid (47 mg, 59% overall yield). LCMS Rₜ: 2.05 min, Analytical HPLC Rₜ: 14.025 min (87% purity), LRMS m/z (ESI⁺): 399.2 [M+2H] (100%), 796.9 [M+H] (60%), HRMS m/z (ESI⁺): calc. for C₄₀H₆₂N₉O₆S ([M+H⁺]) 796.4544, found 796.4556.

### cyclo-cys agr leu ile ile (4-F)phe (SEQ ID NO: 54, Cyclic Peptide #24)

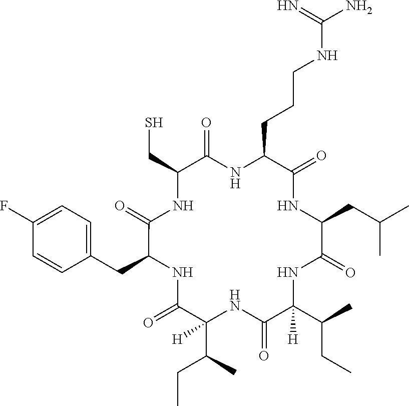 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccc(F)cc2)NC(=O)[C@H]([C@@H](C)CC)NC1=O]

The linear peptide (4-F)phe cys(StBu) agr leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 24 as a white solid (43 mg, 57% overall yield). LCMS Rₜ: 2.32 min, Analytical HPLC Rₜ: 12.686 min (87% purity), LRMS m/z (ESI⁺): 383.1 [M+2H] (100%), 764.8 [M+H] (47%), HRMS m/z (ESI⁺): calc. for C₃₆H₅₉FN₉O₆S ([M+H⁺]) 764.4293, found 764.4286.

### cyclo-cys agr leu ile ile (4-Bz)phe (SEQ ID NO: 55, Cyclic Peptide #25)

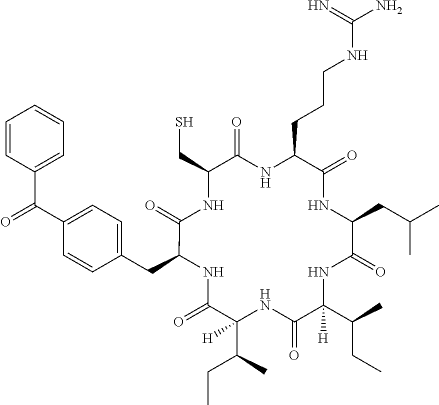 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccc(C(=O)c3ccccc3)cc2)NC(=O)[C@H]([C@@H](C)CC)NC1=O]

The linear peptide (4-Bz)phe cys(StBu) agr leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 25 as a white solid (47 mg, 55% overall yield). LCMS Rₜ: 2.01 min, Analytical HPLC Rₜ: 13.511 min (99% purity), LRMS m/z (ESI⁺): 850.9 [M+H] (100%), 426.1 [M+2H] (90%), HRMS m/z (ESI⁺): calc. for C₄₃H₆₄N₉O₇S ([M+H⁺]) 850.4644, found 850.4659.

### cyclo-cys agr leu ile ile (4-NO₂)phe (SEQ ID NO: 56, Cyclic Peptide #26)

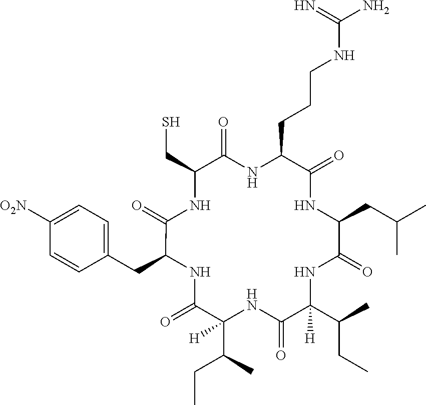 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccc([N+](=O)[O-])cc2)NC(=O)[C@H]([C@@H](C)CC)NC1=O]

The linear peptide (4-NO₂)phe cys(StBu) agr leu ile ile was synthesised from Fmoc-Ile-Wang resin (Novabiochem) on a 0.2 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 26 as a white solid (90 mg, 57% overall yield). LCMS Rₜ: 1.86 min, Analytical HPLC Rₜ: 12.507 min (99% purity), LRMS m/z (ESI⁺): 791.9 [M+H] (100%), 396.6 [M+2H], HRMS m/z (ESI⁺): calc. for C₃₆H₅₉N₁₀O₈S ([M+H⁺]) 791.4238, found 791.4230.

### cyclo-cys agr leu ile ile (4-CN)phe (SEQ ID NO: 57, Cyclic Peptide #27)

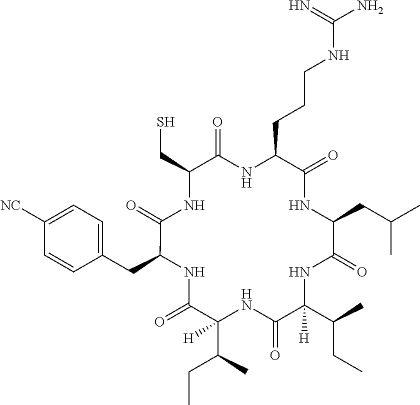 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccc(C#N)cc2)NC(=O)[C@H]([C@@H](C)CC)NC1=O]

The linear peptide (4-CN)phe cys(StBu) agr leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 27 as a white solid (38 mg, 49% overall yield). LCMS Rₜ: 2.19 min, Analytical HPLC Rₜ: 12.002 min (86% purity), LRMS m/z (ESI⁺): 771.8 [M+H] (100%), 386.6 [M+2H] (75%), HRMS m/z (ESI⁺): calc. for C₃₇H₅₉N₁₀O₆S ([M+H⁺]) 771.4334, found 771.4319.

### cyclo-cys agr leu ile ile phg (SEQ ID NO: 58, Cyclic Peptide #28)

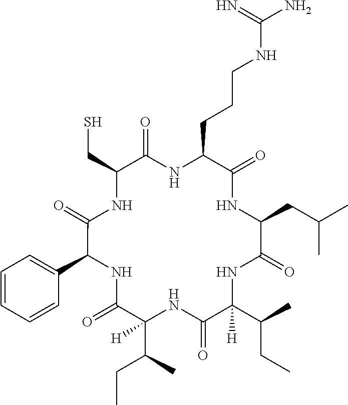 [SMILES: CC[C@H](C)[C@@H]1NC(=O)[C@H](CC(C)C)NC(=O)[C@H](CCCNC(=N)N)NC(=O)[C@H](CS)NC(=O)[C@H](c2ccccc2)NC(=O)[C@H]([C@@H](C)CC)NC1=O]

The linear peptide phg cys(StBu) agr leu ile ile was synthesised from Fmoc-Ile-Wang resin (Novabiochem) on a 0.05 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 28 as a white solid (15 mg, 41% overall yield). LCMS Rₜ: 1.79 min, Analytical HPLC Rₜ: 12.113 min (93% purity), LRMS m/z (ESI⁺): 367.1 [M+2H] (100%), 732.8 [M+H] (40%), HRMS m/z (ESI⁺): calc. for C₃₅H₅₈N₉O₆S ([M+H⁺]) 732.4225, found 732.4230.

### cyclo-cys agr leu ile ile tyr (SEQ ID NO: 60, Cyclic Peptide #29)

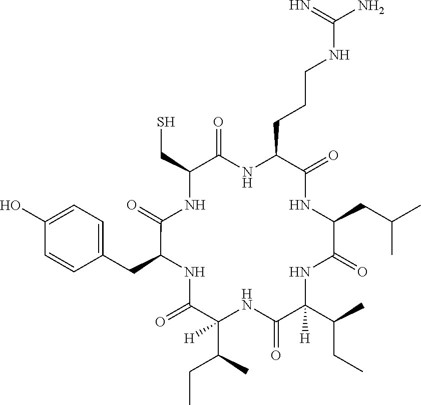 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccc(O)cc2)NC(=O)[C@H]([C@@H](C)CC)NC1=O]

The linear peptide tyr cys(StBu) agr leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 29 as a white solid (50 mg, 66% overall yield). LCMS Rₜ: 1.64 min, Analytical HPLC Rₜ: 10.951 min (85% purity), LRMS m/z (ESI⁺): 382.1 [M+2H] (100%), 762.8 [M+H] (75%), HRMS m/z (ESI⁺): calc. for C₃₆H₆₀N₉O₇S ([M+H]⁺) 762.4336, found 762.4316.

### cyclo-cys agr leu ile ile (me)tyr (SEQ ID NO: 61, Cyclic Peptide #30) [Included for Comparative Purposes Only]

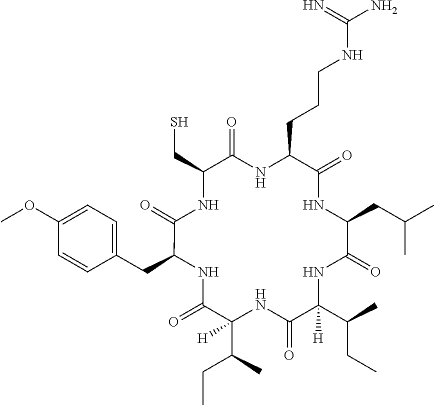 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccc(OC)cc2)NC(=O)[C@H]([C@@H](C)CC)NC1=O]

The linear peptide (me)tyr cys(StBu) agr leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 30 as a white solid (42 mg, 54% overall yield). LCMS Rₜ: 2.25 min, Analytical HPLC Rₜ: 12.411 min (88% purity), LRMS m/z (ESI⁺): 389.1 [M+2H] (100%), 776.8 [M+H] (50%). HRMS m/z (ESI⁺): calc. for C₃₆H₅₉IN₉O₆S ([M+H⁺]) 776.4493, found 776.4475.

### cyclo-cys agr leu ile ile (4-pal) (SEQ ID NO: 62, Cyclic Peptide #31)

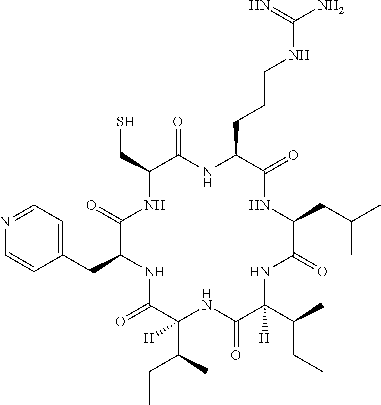 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccncc2)NC(=O)[C@H]([C@@H](C)CC)NC1=O]

The linear peptide (4-pal) cys(StBu) agr leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 31 as a white solid (38 mg, 51% overall yield). LCMS Rₜ: 1.27 min, Analytical HPLC Rₜ: 8.884 min (85% purity), LRMS m/z (ESI⁺): 374.6 [M+2H] (100%), 747.8 [M+H] (5%), HRMS m/z (ESI⁺): calc. for C₃₅H₅₉N₁₀O₆S ([M+H⁺]) 747.4334, found 747.4319.

### cyclo-cys agr leu ile ile D-phe (SEQ ID NO: 59, Cyclic Peptide #32)

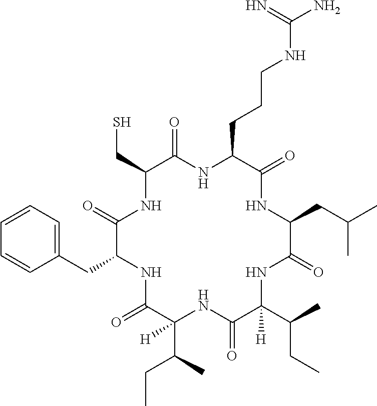 [SMILES: CC[C@H](C)[C@@H]1NC(=O)[C@H](CC(C)C)NC(=O)[C@H](CCCNC(=N)N)NC(=O)[C@H](CS)NC(=O)[C@@H](Cc2ccccc2)NC(=O)[C@H]([C@@H](C)CC)NC1=O]

The linear peptide D-phe cys(StBu) agr leu ile ile was synthesised from Fmoc-Ile-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 32 as a white solid (17 mg, 23% overall yield). LCMS Rₜ: 1.79 min, Analytical HPLC Rₜ: 11.791 min (84% purity), LRMS m/z (ESI⁺): 374.1 [M+2H] (100%), 746.9 [M+H] (90%), HRMS m/z (ESI⁺): calc. for C₃₆H₆₀N₉O₆S ([M+H⁺]) 746.4382, found 746.4391.

### cyclo-cys agr leu ile h-leu phe (SEQ ID NO: 63, Cyclic Peptide #33)

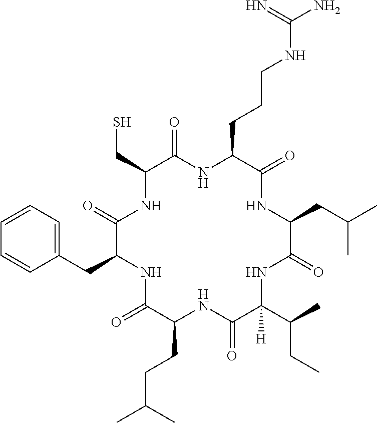 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccccc2)NC(=O)[C@H](CCC(C)C)NC1=O]

The linear peptide ile h-leu phe cys(StBu) arg leu was synthesised from Fmoc-Leu-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 33 as a white solid (31 mg, 41% overall yield). LCMS Rₜ: 1.95 min, Analytical HPLC Rₜ: 13.394 min (99% purity), LRMS m/z (ESI⁺): 381.2 [M+2H] (100%), 760.8 [M+H] (90%), HRMS m/z (ESI⁺): calc. for C₃₇H₆₂N₉O₆S ([M+H⁺]) 760.4538, found 760.4548.

### cyclo-cys agr leu ile (n-leu) phe (SEQ ID NO: 64, Cyclic Peptide #34)

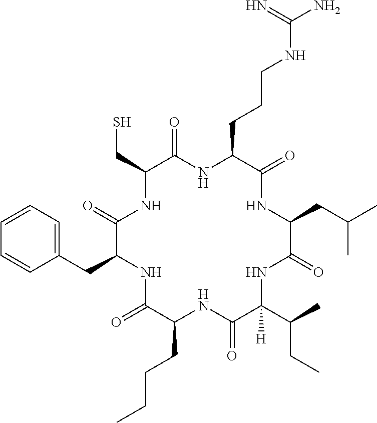 [SMILES: CCCC[C@@H]1NC(=O)[C@H]([C@@H](C)CC)NC(=O)[C@H](CC(C)C)NC(=O)[C@H](CCCNC(=N)N)NC(=O)[C@H](CS)NC(=O)[C@H](Cc2ccccc2)NC1=O]

The linear peptide ile (n-leu) phe cys(StBu) arg leu was synthesised from Fmoc-Leu-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 34 as a white solid (44 mg, 59% overall yield). LCMS Rₜ: 1.87 min, Analytical HPLC Rₜ: 12.706 min (96% purity), LRMS m/z (ESI⁺): 374.1 [M+2H] (100%), 746.8 [M+H] (90%), HRMS m/z (ESI⁺): calc. for C₃₆H₆₀N₉O₆S ([M+H⁺]) 746.4382, found 746.4374.

### cyclo-cys agr leu ile (Nva) phe (SEQ ID NO: 66, Cyclic Peptide #35) [Included for Comparative Purposes Only]

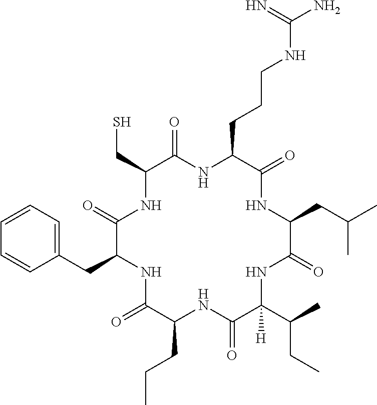 [SMILES: CCC[C@@H]1NC(=O)[C@H]([C@@H](C)CC)NC(=O)[C@H](CC(C)C)NC(=O)[C@H](CCCNC(=N)N)NC(=O)[C@H](CS)NC(=O)[C@H](Cc2ccccc2)NC1=O]

The linear peptide ile (Nva) phe cys(StBu) arg leu was synthesised from Fmoc-Leu-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 35 as a white solid (32 mg, 44% overall yield). LCMS Rₜ: 1.77 min, Analytical HPLC Rₜ: 12.031 min (97% purity), LRMS m/z (ESI⁺): 367.1 [M+2H] (100%), 732.7 [M+H] (75%), HRMS m/z (ESI⁺): calc. for C₃₅H₅₈N₉O₆S ([M+H⁺]) 732.4225, found 732.4220.

### cyclo-cys agr leu ile leu phe (SEQ ID NO: 65, Cyclic Peptide #36)

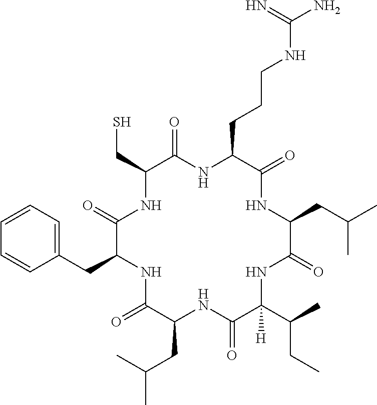 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccccc2)NC(=O)[C@H](CC(C)C)NC1=O]

The linear peptide ile leu phe cys(StBu) arg leu was synthesised from Fmoc-Leu-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 36 as a white solid (43 mg, 57% overall yield). LCMS Rₜ: 1.87 min, Analytical HPLC Rₜ: 12.622 min (99% purity), LRMS m/z (ESI⁺): 746.7 [M+H] (100%), 374.1 [M+2H] (85%), HRMS m/z (ESI⁺): calc. for C₃₆H₆₀N₉O₆S ([M+H⁺]) 746.4382, found 746.4397.

### cyclo-cys agr leu ile val phe (SEQ ID NO: 67, Cyclic Peptide #37) [Included for Comparative Purposes Only]

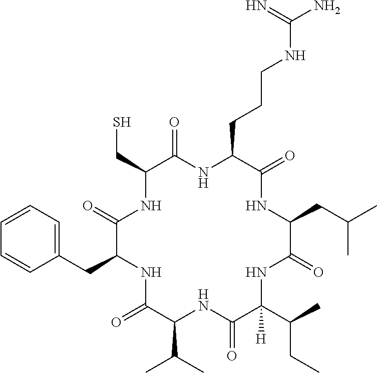 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccccc2)NC(=O)[C@H](C(C)C)NC1=O]

The linear peptide val phe cys(StBu) arg leu ile was synthesised from Fmoc-Ile-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 37 as a white solid (15 mg, 21% overall yield). LCMS Rₜ: 1.79 min, Analytical HPLC Rₜ: 12.149 min (81% purity), LRMS m/z (ESI⁺): 732.6 [M+H] (100%), 367.0 [M+2H] (50%), HRMS m/z (ESI⁺): calc. for C₃₅H₅₈N₉O₆S ([M+H⁺]) 732.4225, found 732.4221.

### cyclo-cys agr leu ile (Aib) phe (Aib)F (SEQ ID NO: 68, Cyclic Peptide #38) [Included for Comparative Purposes Only]

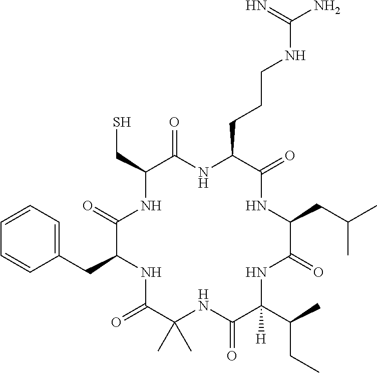 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccccc2)NC(=O)C(C)(C)NC1=O]

The linear peptide (Aib) phe cys(StBu) arg leu ile was synthesised from Fmoc-Ile-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 38 as a white solid (18 mg, 25% overall yield). LCMS Rₜ: 1.63 min, Analytical HPLC Rₜ: 11.154 min (99% purity), LRMS m/z (ESI⁺): 360.2 [M+2H] (100%), 718.8 [M+H] (70%), HRMS m/z (ESI⁺): calc. for C₃₄H₅₆N₉O₆S ([M+H⁺]) 718.4069, found 732.4060.

### cyclo-cys agr leu ile (Abu) phe (SEQ ID NO: 69, Cyclic Peptide #39) [Included for Comparative Purposes Only]

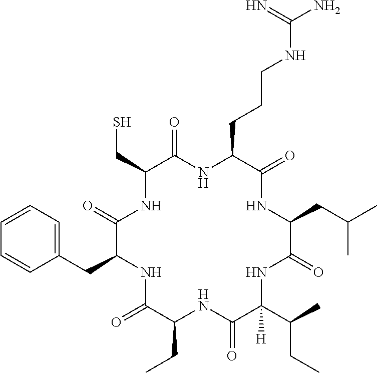 [SMILES: CC[C@@H]1NC(=O)[C@H]([C@@H](C)CC)NC(=O)[C@H](CC(C)C)NC(=O)[C@H](CCCNC(=N)N)NC(=O)[C@H](CS)NC(=O)[C@H](Cc2ccccc2)NC1=O]

The linear peptide ile (Abu) phe cys(StBu) arg leu was synthesised from Fmoc-Leu-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 39 as a white solid (48 mg, 67% overall yield). LCMS Rₜ: 1.69 min, Analytical HPLC Rₜ: 11.382 min (97% purity), LRMS m/z (ESI⁺): 718.7 [M+H] (100%), 360.0 [M+2H] (80%), HRMS m/z (ESI⁺): calc. for C₃₄H₅₆N₉O₆S ([M+H⁺]) 718.4069, found 718.4062.

### cyclo-cys arg leu ile h-leu (4-I)phe (SEQ ID NO: 71, Cyclic Peptide #40)

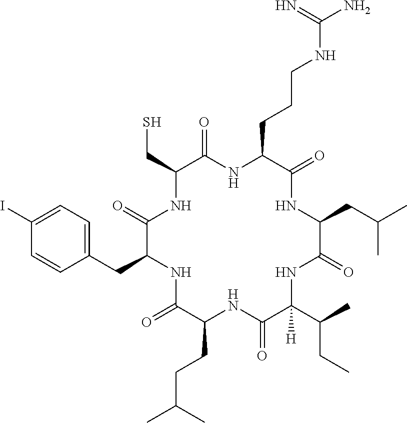 [SMILES: CC[C@H](C)[C@@H]1NC(=O)[C@H](CC(C)C)NC(=O)[C@H](CCCNC(=N)N)NC(=O)[C@H](CS)NC(=O)[C@H](Cc2ccc(I)cc2)NC(=O)[C@H](CCC(C)C)NC1=O]

The linear peptide ile h-leu phe cys(StBu) arg leu was synthesised from Fmoc-Leu-Wang resin (Novabiochem) on a 0.1 mmol scale following general procedure 0. After precipitation from ether, the peptide was dissolved in DMF and cyclised following general procedure 0. The peptide was concentrated, and then deprotected following general procedure 0. The product was purified by RP-HPLC (method A) and lyophilised to obtain 40 as a white solid (47 mg, 53% overall yield). LCMS Rₜ: 2.28 min, Analytical HPLC Rₜ: 14.459 min (98% purity), LRMS m/z (ESI⁺): 886.8 [M+H] (100%), 444.0 [M+2H] (80%), HRMS m/z (ESI⁺): calc. for C₃₇H₆₁IN₉O₆S ([M+H⁺]) 886.3505, found 886.3525.

1. A cyclic peptide, wherein the cyclic peptide has the following sequence: 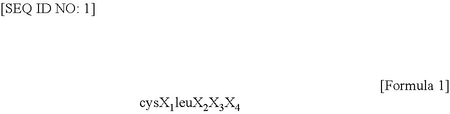
wherein:
cys is cysteine;
X₁ is selected from the group comprising or consisting of arginine (arg) or lysine (lys);
leu is leucine;
X₂ is selected from the group comprising or consisting of leu or isoleucine (ile);
X₃ is: 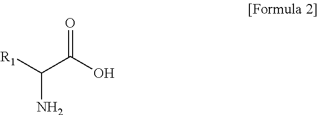
wherein:
R₁ is C₂-C₆ linear or branched alkyl;
X₄ is: 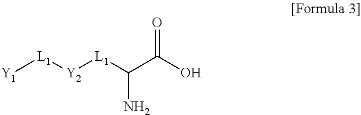
wherein:
each L₁ is independently C₁-C₃ alkyl, a direct bond or C═O;
Y₂ is a 5-12 membered aromatic group optionally substituted with halogen, C₁-C₃ alkyl, C₁-C₃ haloalkyl, NO₂, CN, OH or O—(C₁-C₃ alkyl); and
Y₁ is H or a 5-12 membered aromatic group optionally substituted with halogen, C₁-C₃ alkyl, C₁-C₃ haloalkyl, NO₂, CN, OH or O—(C₁-C₃ alkyl);
provided that:
when X₁ is arg, X₂ is leu and X₃ is ile; X₄ is not phenylalanine (phe), phe(4-Cl), phe(4-F), phe(4-NO₂), or phe(4-CN); and
when X₁ is lysine (lys), X₂ is leu and X₃ is ile; X₄ is not phe, phe(4-Cl), phe(4-F), phe(4-NO2), or phe(4-CN);
wherein phe, phe(4-Cl), phe(4-F), phe(4-NO2), and phe(4-CN) have the following structures:

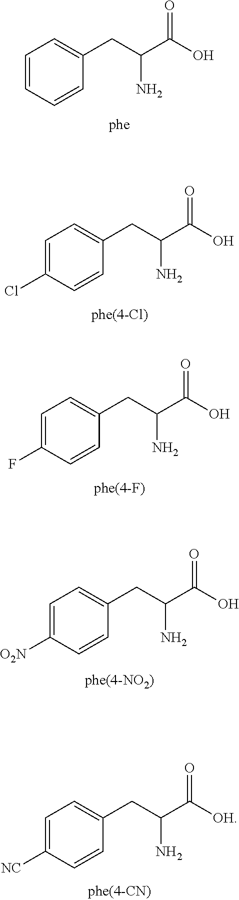 [SMILES: NC(Cc1ccccc1)C(=O)O ; NC(Cc1ccc(Cl)cc1)C(=O)O ; NC(Cc1ccc(F)cc1)C(=O)O ; NC(Cc1ccc([N+](=O)[O-])cc1)C(=O)O ; N#Cc1ccc(CC(N)C(=O)O)cc1]

2. The cyclic peptide according to claim 1, wherein:
a) R₁ is a C₄-C₅ linear or branched alkyl, preferably wherein R₁ is selected from the group comprising or consisting of —C₄H₉, —CH₂CH(CH₃)₂, —CH(CH₃)(C₂H₅), —CH₂CH₂CH(CH₃)₂;
b) L₁ is a C₁-C₂ alkyl, a direct bond or C═O;
c) Y₂ is a 6-10 membered aromatic group, preferably wherein Y₂ is selected from phenyl or naphthyl;
d) Y₂ is substituted with a substituent from the group comprising or consisting of F, Cl, I, CF₃, CN, OH, OMe, and NO₂;
e) Y₂ is unsubstituted;
f) Y₁ is H or phenyl, preferably wherein said phenyl is unsubstituted;
g) X₃ is selected from the group comprising or consisting of leu, ile, h-leu, or n-leu, or abu wherein n-leu has the following structure: 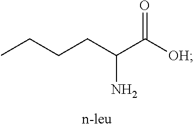
h) X₄ is selected from the group comprising or consisting of phe, phe(4-I), phe(4-Br), phe(4-CF₃), phe(4-Cl), h-phe, ala(1-naph), phe(4-F), phe(4-Bz), phe(4-NO₂), phe(4-CN), gly(Ph), D-phe, and tyr(OMe) having the following structures: 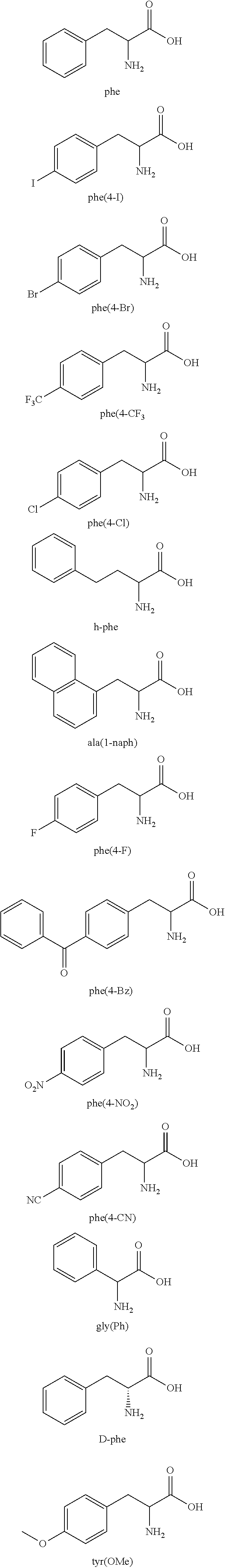
i) wherein X₄ is selected from the group comprising or consisting of: phe(4-I) and phe(4-Bz), preferably phe(4-I); or
j) wherein X₄ is selected from the group comprising or consisting of phe, phe(4-I), phe(4-Br), phe(4-CF₃), phe(4-Cl), h-phe, ala(1-naph), phe(4-F), phe(4-Bz), phe(4-NO₂), 4-Pal or phe(4-CN).

3.-9. (canceled)

10. The cyclic peptide according to claim 1, wherein the peptide has the sequence:
[SEQ ID NO: 2]
cys arg leu ile ile X₄,
[SEQ ID NO: 3]
cys arg leu leu ile X₄,
[SEQ ID NO: 4]
cys lys leu ile ile X₄,
or
[SEQ ID NO: 5]
cys lys leu leu ile X₄.
optionally wherein the peptide has the sequence:
[SEQ ID NO: 6]
cys arg leu ile ile phe;
or
[SEQ ID NO: 7]
cys lys leu leu ile phe.

11.-13. (canceled)

14. The cyclic peptide according to claim 1, wherein the cyclic peptide has a sequence selected from the group comprising or consisting of:
[SEQ ID NO: 8]
cys arg leu ile ile phe,
[SEQ ID NO: 9]
cys arg leu ile ile phe(4-Br),
[SEQ ID NO: 10]
cys arg leu ile ile phe(4-CF3),
[SEQ ID NO: 11]
cys arg leu ile ile phe(4-Cl),
[SEQ ID NO: 12]
cys arg leu ile ile (h-phe),
[SEQ ID NO: 13]
cys arg leu ile ile (ala(1-naph)),
[SEQ ID NO: 14]
cys arg leu ile ile phe(4-F),
[SEQ ID NO: 15]
cys arg leu ile ile phe(4-Bz),
[SEQ ID NO: 16]
cys arg leu ile ile phe(4-NO₂),
[SEQ ID NO: 17]
cys arg leu ile ile phe(4-CN),
[SEQ ID NO: 18]
cys arg leu ile ile(gly(Ph)),
[SEQ ID NO: 19]
cys arg leu ile ile (D-phe),
[SEQ ID NO: 20]
cys arg leu ile ile tyr,
[SEQ ID NO: 48]
cys arg leu ile ile phe(4-I),
and
[SEQ ID NO: 62]
cys arg leu ile ile 4-Pal,
or optionally
has a sequence selected from the group comprising or consisting:
[SEQ ID NO: 8]
cys arg leu ile ile phe,
[SEQ ID NO: 9]
cys arg leu ile ile phe(4-Br),
[SEQ ID NO: 10]
cys arg leu ile ile phe(4-CF3),
[SEQ ID NO: 11]
cys arg leu ile ile phe(4-Cl),
[SEQ ID NO: 12]
cys arg leu ile ile (h-phe),
[SEQ ID NO: 13]
cys arg leu ile ile (ala(1-naph)),
[SEQ ID NO: 14]
cys arg leu ile ile phe(4-F),
[SEQ ID NO: 15]
cys arg leu ile ile phe(4-Bz),
[SEQ ID NO: 16]
cys arg leu ile ile phe(4-NO₂),
[SEQ ID NO: 17]
cys arg leu ile ile phe(4-CN),
[SEQ ID NO: 48]
cys arg leu ile ile phe(4-I),
and
[SEQ ID NO: 62]
cys arg leu ile ile 4-Pal
or optionally
has a sequence selected from the group comprising or consisting:
[SEQ ID NO: 8]
cys arg leu ile ile phe,
[SEQ ID NO: 9]
cys arg leu ile ile phe(4-Br),
[SEQ ID NO: 10]
cys arg leu ile ile phe(4-CF3),
[SEQ ID NO: 11]
cys arg leu ile ile phe(4-Cl),
[SEQ ID NO: 12]
cys arg leu ile ile (h-phe),
[SEQ ID NO: 13]
cys arg leu ile ile (ala(1-naph)),
[SEQ ID NO: 14]
cys arg leu ile ile phe(4-F),
[SEQ ID NO: 15]
cys arg leu ile ile phe(4-Bz),
[SEQ ID NO: 16]
cys arg leu ile ile phe(4-NO₂).

15.-17. (canceled)

18. The cyclic peptide according to claim 1, wherein the cyclic peptide has a sequence selected from the group comprising or consisting of:
[SEQ ID NO: 63]
cys arg leu ile h-leu phe
[SEQ ID NO: 23]
cys arg leu ile leu phe,
and
[SEQ ID NO: 69]
cys arg leu ile abu phe,
preferably cys arg leu ile h-leu phe [SEQ ID NO: 63].

19. The cyclic peptide according to claim 1, wherein the peptide has the sequence
cys arg leu ile h-leu phe(4-I) [SEQ ID NO: 24] or the sequence cys arg leu ile h-leu (4-I)Phe [SEQ ID NO: 71].

20. The cyclic peptide according to claim 1, wherein any one or more of cys, X₁, leu, X₂, X₃, or X₄ is a D amino acid.

21. The cyclic peptide according to claim 1, wherein any one or more of cys, X₁, leu, X₂, X₃, X₄ is an L amino acid.

22. The cyclic peptide according to claim 1, wherein the cyclic peptide is capable of:
a) binding to HIF-2, optionally capable of binding to recombinantly expressed PAS-B domain of HIF-2α:
b) binding to HIF-1, optionally capable of binding to recombinantly expressed PAS-B domain of HIF-1α;
c) capable of binding to HIF-1 and HIF-2, optionally capable of binding to recombinantly expressed PAS-B domain of HIF-2α and HIF-1α;
d) binding to HIF-1 and HIF-2, optionally capable of binding to recombinantly expressed PAS-B domain of HIF-2α and HIF-1α; or
e) binding to HIF-1 and HIF-2 with a similar affinity, optionally capable of binding to recombinantly expressed PAS-B domain of HIF-2α and HIF-1α with similar affinity,
wherein a cyclic peptide binds to HIF-1 and HIF-2, or to recombinantly expressed PAS-B domain of HIF-2α and HIF-1α, with a similar affinity when the difference in affinity of binding to HIF-1 and HIF-2, or to recombinantly expressed PAS-B domain of HIF-2α and HIF-1α is:
less than 60 μM, 55 μM, 50 μM, 45 μM, 40 μM, 35 μM, 30 μM, 25 μM, 20 μM, 18 μM, 16 μM, 15 μM, 14 μM, 13 μM, 12 μM, 11 μM, 10 μM, 9 μM, 8 μM, 7 μM, 6 μM, 5 μM, 4 μM, 3 μM, 2 μM, 1 μM, 0.8 μM, 0.6 μM, 0.5 μM, 0.4 μM, 0.3 μM, 0.2 μM, 0.1 μM; and/or
between 0.1 μM and 10 μM, 0.2 μM and 9 μM, 0.3 μM and 8 μM, 0.4 μM and 7 μM, 0.5 μM and 6 μM, 0.6 and 5.5 μM, 0.7 and 5 μM, 0.8 and 4.5 μM, 0.9 μM and 4 μM, 1 and 3.5 μM, 1.25 μM and 3 μM, 1.5 μM and 2.75 μM, 1.75 μM and 2.5 μM, 2 μM and 2.25 μM; and/or
between 0.1 μM and 60 μM, 0.2 μM and 55 μM, 0.3 μM and 50 μM, 0.4 μM and 45 μM, 0.5 μM and 40 μM, 0.6 μM and 35 μM, 0.7 μM and 30 μM, 0.8 μM and 25 μM, 0.9 μM and 20 μM, 1 μM and 18 μM, 2 μM and 16 μM, 3 μM and 15 μM, 4 μM and 14 μM, 5 μM and 13 μM, 6 μM and 12 μM, 7 μM and 11 μM, 8 μM and 10 μM,
optionally wherein the affinity is determined against the recombinantly expressed PAS-B domain of both HIF-1α and 2α using microscale thermophoresis.

23.-26. (canceled)

27. The cyclic peptide according to claim 1, wherein the cyclic peptide prevents or reduces the hypoxia induced expression from a promoter that comprises one or more hypoxia-responsive elements under hypoxic conditions, optionally to less than 90%, 80%, 70%, 60%, 50%, 40%, 30%, 20%, 10%, 5%, 4%, 3%, 2%, 1% of the expression obtained in the absence of the cyclic peptide.

28. (canceled)

29. The cyclic peptide according to claim 1, wherein the cyclic peptide disrupts the interaction between HIF-1a and HIF-1b, optionally wherein the interaction is assessed by proximity ligation assay, optionally in MCF-7 cells.

30. A polynucleotide comprising or consisting of a sequence that encodes a cyclic peptide according to claim 1.

31. A nucleic acid vector comprising the polynucleotide according to claim 30.

32. A cell comprising the polynucleotide according to claim 30.

33. A viral vector comprising the polynucleotide according to claim 30.

34. A pharmaceutical composition comprising one or more of the cyclic peptides according to claims 1.

35.-36. (canceled)

37. The method according to claim 42, wherein the cancer is selected from the group comprising or consisting of:
acute lymphoblastic leukemia (ALL), Acute myeloid leukemia, Adrenocortical carcinoma, AIDS-related cancers, AIDS-related lymphoma, Anal cancer, Appendix cancer, Astrocytoma, childhood cerebellar or cerebral, Basal-cell carcinoma, Bile duct cancer, extrahepatic (see cholangiocarcinoma), Bladder cancer, Bone tumor, osteosarcoma/malignant fibrous histiocytoma, Brainstem glioma, Brain cancer, Brain tumor, cerebellar astrocytoma, Brain tumor, cerebral astrocytoma/malignant glioma, Brain tumor, ependymoma, Brain tumor, medulloblastoma, Brain tumor, supratentorial primitive neuroectodermal tumors, Breast cancer, Bronchial adenomas/carcinoids, Burkitt's lymphoma, Carcinoid tumor, childhood, Carcinoid tumor, gastrointestinal, Carcinoma of unknown primary, Cerebellar astrocytoma, Cerebral astrocytoma/malignant glioma, Cervical cancer, Chondrosarcoma, Chronic lymphocytic leukemia, Chronic myelogenous leukemia, Chronic myeloproliferative disorders, Colon cancer, Cutaneous T-cell lymphoma, Desmoplastic small round cell tumor, Endometrial cancer, Ependymoma, Esophageal cancer, Ewing's sarcoma, Extracranial germ cell tumor, Extragonadal germ cell tumor, Extrahepatic bile duct cancer, intraocular melanoma, retinoblastoma, Gallbladder cancer, Gastric (stomach) cancer, Gastrointestinal carcinoid tumor, Gastrointestinal stromal tumor (GIST), Germ cell tumor: extracranial, extragonadal, or ovarian, Gestational trophoblastic tumor, Glioma of the brain stem, Glioma, childhood cerebral astrocytoma, Glioma, childhood visual pathway and hypothalamic, Gastric carcinoid, Hairy cell leukemia, Hepatocellular (liver) cancer, Hodgkin lymphoma, Hypopharyngeal cancer, Hypothalamic and visual pathway glioma, childhood, Intraocular melanoma, Islet cell carcinoma (endocrine pancreas), Kaposi sarcoma, Kidney cancer (renal cell cancer), Laryngeal cancer, Leukaemias, Leukaemia, acute lymphoblastic (also called acute lymphocytic leukaemia), Leukaemia, acute myeloid (also called acute myelogenous leukemia), Leukaemia, chronic lymphocytic (also called chronic lymphocytic leukemia), Leukemia, chronic myelogenous (also called chronic myeloid leukemia), Leukemia, hairy cell, Lip and oral cavity cancer, Liposarcoma, Liver cancer (primary), Lung cancer, non-small cell, Lung cancer, small cell, Lymphomas, Lymphoma, AIDS-related, Lymphoma, Burkitt, Lymphoma, cutaneous T-Cell, Lymphoma, Hodgkin, Lymphomas, Non-Hodgkin, Lymphoma, primary central nervous system, Macroglobulinemia, Waldenstrom, Malignant fibrous histiocytoma of bone/osteosarcoma, Medulloblastoma, Melanoma, Merkel cell cancer, Mesothelioma, Mouth cancer, Multiple endocrine neoplasia syndrome, Multiple myeloma/plasma cell neoplasm, Mycosis fungoides, Myelodysplastic syndromes Myelodysplastic/myeloproliferative diseases, Myelogenous leukemia, chronic Myeloid leukemia, adult acute, Myeloid leukemia, childhood acute, Myeloma, multiple (cancer of the bone-marrow), Myeloproliferative disorders, chronic, Myxoma, Nasal cavity and paranasal sinus cancer, Nasopharyngeal carcinoma, Neuroblastoma, Non-Hodgkin lymphoma, Non-small cell lung cancer, Oligodendroglioma, Oral cancer, Oropharyngeal cancer, Osteosarcoma/malignant fibrous histiocytoma of bone, Ovarian cancer, Ovarian epithelial cancer (surface epithelial-stromal tumor), Ovarian germ cell tumor, Ovarian low malignant potential tumor, Pancreatic cancer, Pancreatic cancer, islet cell, Paranasal sinus and nasal cavity cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, childhood, Pituitary adenoma, Plasma cell neoplasia/Multiple myeloma, Pleuropulmonary blastoma, Primary central nervous system lymphoma, Prostate cancer, Rectal cancer, Renal cell carcinoma, Renal pelvis and ureter, transitional cell cancer, Rhabdomyosarcoma, childhood, Salivary gland cancer, Sarcoma, Ewing family of tumors, Sarcoma, Kaposi, Sarcoma, soft tissue, Sarcoma, uterine, Sezary syndrome, Skin cancer (non-melanoma), Skin cancer (melanoma), Skin carcinoma, Merkel cell, Small cell lung cancer, Small intestine cancer, Soft tissue sarcoma, Squamous cell carcinoma—see skin cancer (non-melanoma), Squamous neck cancer with occult primary, metastatic, Stomach cancer, Supratentorial primitive neuroectodermal tumor, childhood, T-Cell lymphoma, cutaneous, Testicular cancer, Throat cancer, Thymoma, Thymoma and thymic carcinoma, Thyroid cancer, Thyroid cancer, Transitional cell cancer of the renal pelvis and ureter, Trophoblastic tumor, gestational, Unknown primary site, Ureter and renal pelvis, transitional cell cancer, Urethral cancer, Uterine cancer, endometrial, Uterine sarcoma, Vaginal cancer, Visual pathway and hypothalamic glioma, Vulvar cancer, Waldenstrom macroglobulinemia and/or Wilms tumor (kidney cancer).

38. (canceled)

39. The cyclic peptide according to claim 1, wherein the cyclic peptide is formulated with one or more further therapeutic agents, optionally one or more further anti-cancer therapeutic agents.

40. The method according to claim 42, wherein the cyclic peptide is administered as part of a combination therapy.

41. A method for the treatment or prevention of disease, wherein the method comprises administration of one or more cyclic peptides according claim 1 to a patient in need thereof.

42. A method for the treatment or prevention of cancer, wherein the method comprises administration of one or more cyclic peptides according to claim 1 to a patient in need thereof.

43. A method for the treatment or prevention of a disease, disorder or condition:
that experiences a hypoxic environment and requires the typical hypoxia response for maintenance;
that is treatable or preventable by inhibition of dimerization of HIF-1a with HIF1-b and HIF2a with HIF1b and/or inhibits the activity of HIF-1 and HIF-2 and/or HIF-1 or HIF-2 signalling; and/or
in which it is desirable to repress hypoxia induced gene expression,
wherein the method comprises administration of one or more cyclic peptides according to claim 1 to a patient in need thereof.

44.-46. (canceled)

47. A kit comprising one or more of
a cyclic peptide according to claim 1.


SEQUENCE LISTING (71 sequences)

SEQ ID NO: 1 - protein, 6 aa, Artificial Sequence
  Consensus sequence
  SITE 2: Wherein Xaa is selected from the group comprising or consisting of arginine or lysine
  SITE 4: Wherein Xaa is selected from the group comprising or consisting of leucine or isoleucine; provided that when Xaa at position 2 is arginine or lysine, Xaa at position 4 is leucine
  SITE 5: Wherein Xaa is Formula 2; provided that when Xaa at position 2 is arginine or lysine, Xaa at position 5 is isoleucine
  SITE 6: Wherein Xaa is Formula 3; provided that when Xaa at position 2 is arginine or lysine, Xaa at position 6 is not phenylalanine (phe), phe(4-Cl), phe(4-F), phe(4-NO2), or phe(4-CN)
CXLXXX

SEQ ID NO: 2 - protein, 6 aa, Artificial Sequence
  Peptide
  SITE 6: Wherein Xaa is Formula 3
CRLIIX

SEQ ID NO: 3 - protein, 6 aa, Artificial Sequence
  Peptide
  SITE 6: Wherein Xaa is Formula 3, but is not phenylalanine (phe), phe(4-Cl), phe(4-F), phe(4-NO2), or phe(4-CN)
CRLLIX

SEQ ID NO: 4 - protein, 6 aa, Artificial Sequence
  Peptide
  SITE 6: Wherein Xaa is Formula 3
CKLIIX

SEQ ID NO: 5 - protein, 6 aa, Artificial Sequence
  Peptide
  SITE 6: Wherein Xaa is Formula 3, but is not phenylalanine (phe), phe(4-Cl), phe(4-F), phe(4-NO2), or phe(4-CN)
CKLLIX

SEQ ID NO: 6 - protein, 6 aa, Artificial Sequence
  Cyclic peptide #4
CRLIIF

SEQ ID NO: 7 - protein, 6 aa, Artificial Sequence
  Cyclic peptide #5
CKLLIF

SEQ ID NO: 8 - protein, 6 aa, Artificial Sequence
  Cyclic peptide
CRLIIF

SEQ ID NO: 9 - protein, 6 aa, Artificial Sequence
  Cyclic peptide
  SITE 6: Wherein Xaa is 4-bromophenylalanine (phe(4-Br))
CRLIIX

SEQ ID NO: 10 - protein, 6 aa, Artificial Sequence
  Cyclic peptide
  SITE 6: Wherein Xaa is 4-triflurormethylphenyalinine (phe(4-CF3))
CRLIIX

SEQ ID NO: 11 - protein, 6 aa, Artificial Sequence
  Cyclic peptide
  SITE 6: Wherein Xaa is 4-chlorophenylalanine (phe(4-Cl))
CRLIIX

SEQ ID NO: 12 - protein, 6 aa, Artificial Sequence
  Cyclic peptide
  SITE 6: Wherein Xaa is homophenylalinine (h-phe)
CRLIIX

SEQ ID NO: 13 - protein, 6 aa, Artificial Sequence
  Cyclic peptide
  SITE 6: Wherein Xaa is 1-naphthalenealanine (ala(1-naph))
CRLIIX

SEQ ID NO: 14 - protein, 6 aa, Artificial Sequence
  Cyclic peptide
  SITE 6: Wherein Xaa is 4-fluorophenylalanine (phe(4-F))
CRLIIX

SEQ ID NO: 15 - protein, 6 aa, Artificial Sequence
  Cyclic peptide
  SITE 6: Wherein Xaa is 4-benzoylphenyalinine (phe(4-Bz))
CRLIIX

SEQ ID NO: 16 - protein, 6 aa, Artificial Sequence
  Cyclic peptide
  SITE 6: Wherein Xaa is 4-nitrophenylalanine (phe(4-NO2))
CRLIIX

SEQ ID NO: 17 - protein, 6 aa, Artificial Sequence
  Cyclic peptide
  SITE 6: Wherein Xaa is 4-cyanophenylalanine (phe(4-CN))
CRLIIX

SEQ ID NO: 18 - protein, 6 aa, Artificial Sequence
  Cyclic peptide
  SITE 6: Wherein Xaa is phenylglycine (gly(Ph))
CRLIIX

SEQ ID NO: 19 - protein, 6 aa, Artificial Sequence
  Cyclic peptide
  SITE 6: Wherein Xaa is D-phenylalinine (D-Phe)
CRLIIX

SEQ ID NO: 20 - protein, 6 aa, Artificial Sequence
  Cyclic peptide
CRLIIY

SEQ ID NO: 21-22 - skipped (2 entries)

SEQ ID NO: 23 - protein, 6 aa, Artificial Sequence
  Cyclic peptide
CRLILF

SEQ ID NO: 24 - skipped

SEQ ID NO: 25 - protein, 6 aa, Artificial Sequence
  Cyclic peptide #1
CKLIIF

SEQ ID NO: 26 - protein, 6 aa, Artificial Sequence
  Cyclic peptide #2
CRLLIF

SEQ ID NO: 27 - protein, 6 aa, Artificial Sequence
  Cyclic peptide #3
CRVIIF

SEQ ID NO: 28 - protein, 6 aa, Artificial Sequence
  Cyclic peptide
CLLFVY

SEQ ID NO: 29 - protein, 6 aa, Artificial Sequence
  Cyclic peptide
CAAAAA

SEQ ID NO: 30 - protein, 6 aa, Artificial Sequence
  Cyclic peptide #9
ARLIIF

SEQ ID NO: 31 - protein, 6 aa, Artificial Sequence
  Cyclic peptide #10
CALIIF

SEQ ID NO: 32 - protein, 6 aa, Artificial Sequence
  Cyclic peptide #11
CRAIIF

SEQ ID NO: 33 - protein, 6 aa, Artificial Sequence
  Cyclic peptide #6
CRLAIF

SEQ ID NO: 34 - protein, 6 aa, Artificial Sequence
  cyclic peptide #7
CRLIAF

SEQ ID NO: 35 - protein, 6 aa, Artificial Sequence
  cyclic peptide #8
CRLIIA

SEQ ID NO: 36 - protein, 6 aa, Artificial Sequence
  Cyclic peptide #12a
  SITE 2: Wherein Xaa is penicillamine (pen)
FXRLII

SEQ ID NO: 37 - protein, 6 aa, Artificial Sequence
  Cyclic peptide #12b
  SITE 4: Wherein Xaa is 2-pyridinethiolpenicillamine (pen(S-Py))
IIFXRL

SEQ ID NO: 38 - protein, 6 aa, Artificial Sequence
  SITE 1: Wherein Xaa is 2-pyridinethiolpenicillamine (pen(S-Py))
  Cyclic peptide #12c
XRLIIF

SEQ ID NO: 39 - protein, 6 aa, Artificial Sequence
  SITE 1: Wherein Xaa is penicillamine (pen)
  Cyclic peptide #12d
XRLIIF

SEQ ID NO: 40 - protein, 6 aa, Artificial Sequence
  Cyclic peptide #13a
  SITE 2: Wherein Xaa is homocysteine (h-cys)
FXRLII

SEQ ID NO: 41 - protein, 6 aa, Artificial Sequence
  Cyclic peptide #13b
  SITE 4: Wherein Xaa is 2-pyridinethiolhomocysteine (h-cys(S-Py))
IIFXRL

SEQ ID NO: 42 - protein, 6 aa, Artificial Sequence
  SITE 1: Wherein Xaa is 2-pyridinethiolhomocysteine (h-cys(S-Py))
  Cyclic peptide #13c
XRLIIF

SEQ ID NO: 43 - protein, 6 aa, Artificial Sequence
  SITE 1: Wherein Xaa is homocysteine (h-cys)
  Cyclic peptide #13d
XRLIIF

SEQ ID NO: 44 - protein, 6 aa, Artificial Sequence
  Cyclic peptide #14
MRLIIF

SEQ ID NO: 45 - protein, 6 aa, Artificial Sequence
  Cyclic peptide #15
SRLIIF

SEQ ID NO: 46 - protein, 6 aa, Artificial Sequence
  Cyclic peptide #16
TRLIIF

SEQ ID NO: 47 - protein, 6 aa, Artificial Sequence
  SITE 1: Wherein Xaa is methylcysteine (cys(Me))
  Cyclic peptide #17
XRLIIF

SEQ ID NO: 48 - protein, 6 aa, Artificial Sequence
  Cyclic peptide #18
  SITE 6: Wherein Xaa is 4-iodophenylalanine (phe(4-I))
CRLIIX

SEQ ID NO: 49 - protein, 6 aa, Artificial Sequence
  Cyclic peptide #19
  SITE 6: Wherein Xaa is 4-bromophenylalanine (phe(4-Br))
CRLIIX

SEQ ID NO: 50 - protein, 6 aa, Artificial Sequence
  Cyclic peptide #20
  SITE 6: Wherein Xaa is 4-triflurormethylphenyalinine (phe(4-CF3))
CRLIIX

SEQ ID NO: 51 - protein, 6 aa, Artificial Sequence
  Cyclic peptide #21
  SITE 6: Wherein Xaa is 4-chlorophenylalanine (phe(4-Cl))
CRLIIX

SEQ ID NO: 52 - protein, 6 aa, Artificial Sequence
  Cyclic peptide #22
  SITE 6: Wherein Xaa is homophenylalinine (h-phe)
CRLIIX

SEQ ID NO: 53 - protein, 6 aa, Artificial Sequence
  Cyclic peptide #23
  SITE 6: Wherein Xaa is 1-naphthalenealanine (ala(1-naph))
CRLIIX

SEQ ID NO: 54 - protein, 6 aa, Artificial Sequence
  Cyclic peptide #24
  SITE 6: Wherein Xaa is 4-fluorophenylalanine (phe(4-F))
CRLIIX

SEQ ID NO: 55 - protein, 6 aa, Artificial Sequence
  Cyclic peptide #25
  SITE 6: Wherein Xaa is 4-benzoylphenyalinine (phe(4-Bz))
CRLIIX

SEQ ID NO: 56 - protein, 6 aa, Artificial Sequence
  Cyclic peptide #26
  SITE 6: Wherein Xaa is 4-nitrophenylalanine (phe(4-NO2))
CRLIIX

SEQ ID NO: 57 - protein, 6 aa, Artificial Sequence
  Cyclic peptide #27
  SITE 6: Wherein Xaa is 4-cyanophenylalanine (phe(4-CN))
CRLIIX

SEQ ID NO: 58 - protein, 6 aa, Artificial Sequence
  Cyclic peptide #28
  SITE 6: Wherein Xaa is phenylglycine (gly(Ph))
CRLIIX

SEQ ID NO: 59 - protein, 6 aa, Artificial Sequence
  Cyclic peptide #32
  SITE 6: Wherein Xaa is D-phenylalinine (D-Phe)
CRLIIX

SEQ ID NO: 60 - protein, 6 aa, Artificial Sequence
  Cyclic peptide #29
CRLIIY

SEQ ID NO: 61 - protein, 6 aa, Artificial Sequence
  Cyclic peptide #30
  SITE 6: Wherein Xaa is O-methyltyrosine (tyr(OMe))
CRLIIX

SEQ ID NO: 62 - protein, 6 aa, Artificial Sequence
  Cyclic peptide #31
  SITE 6: Wherein Xaa is 3-pyridin-4-ylalanine (ala(4-Py) or 4-Pal)
CRLIIX

SEQ ID NO: 63 - protein, 6 aa, Artificial Sequence
  Cyclic peptide #33
  SITE 5: Wherein Xaa is homoleucine (h-leu)
CRLIXF

SEQ ID NO: 64 - protein, 6 aa, Artificial Sequence
  Cyclic peptide #34
  SITE 5: Wherein Xaa is norleucine (n-leu)
CRLIXF

SEQ ID NO: 65 - protein, 6 aa, Artificial Sequence
  Cyclic peptide #36
CRLILF

SEQ ID NO: 66 - protein, 6 aa, Artificial Sequence
  Cyclic peptide #35
  SITE 5: Wherein Xaa is norvaline (n-val)
CRLIXF

SEQ ID NO: 67 - protein, 6 aa, Artificial Sequence
  Cyclic peptide #37
CRLIVF

SEQ ID NO: 68 - protein, 6 aa, Artificial Sequence
  Cyclic peptide #38
  SITE 5: Wherein Xaa is 2-aminoisobutyric acid (aib)
CRLIXF

SEQ ID NO: 69 - protein, 6 aa, Artificial Sequence
  Cyclic peptide #39
  SITE 5: Wherein Xaa is 2-aminobutanoic acid (abu)
CRLIXF

SEQ ID NO: 70 - protein, 6 aa, Artificial Sequence
  Cyclic peptide
  SITE 2..6: Wherein Xaa is unknown
CXXXXX

SEQ ID NO: 71 - protein, 6 aa, Artificial Sequence
  Cyclic peptide #40
  SITE 5: Wherein Xaa is homoleucine (h-leu)
  SITE 6: Wherein Xaa is 4-iodophenylalanine (phe(4-I))
CRLIXX